(19) United States
(12) Patent Application Publication
(10) Pub. No.: US 2021/0238668 A1
(43) Pub. Date: Aug. 5, 2021

(54) BITERMINAL DNA FRAGMENT TYPES IN CELL-FREE SAMPLES AND USES THEREOF

(71) Applicants: The Chinese University of Hong Kong, Shatin (HK); GRAIL, Inc., Menlo Park, CA (US)

(72) Inventors: Yuk-Ming Dennis Lo, Homantin (CN); Rossa Wai Kwun Chiu, Shatin (CN); Diana Siao Cheng Han, Tai Po (CN); Meng Ni, Tai Po (CN)

(21) Appl. No.: 17/144,021

(22) Filed: Jan. 7, 2021

Related U.S. Application Data
(60) Provisional application No. 62/958,676, filed on Jan. 8, 2020.

(51) Int. Cl. C12Q 1/6869; C12Q 1/6886

(52) U.S. Cl. CPC C12Q 1/6869; C12Q 2600/112; C12Q 1/6886

(57) ABSTRACT

The present disclosure describes techniques for measuring quantities (e.g., relative frequencies) of end motif pairs of cell-free DNA fragments in a biological sample of an organism for measuring a property of the sample (e.g., fractional concentration of clinically-relevant DNA) and/or determining a pathology of the organism based on such measurements. Different tissue types exhibit different patterns for the relative frequencies of the end motif pairs. The present disclosure provides various uses for measurements of the relative frequencies of end motif pairs of cell-free DNA, e.g., in mixtures of cell-free DNA from various tissues. DNA from certain tissue(s) may be referred to as clinically-relevant DNA.

### CROSS-REFERENCES TO RELATED APPLICATION

This application is a nonprovisional of and claims the benefit of U.S. Provisional patent Application No. 62/958,676, entitled “Biterminal Analysis For Cancer Screening,” filed on Jan. 8, 2020, which is herein incorporated by reference in its entirety for all purposes.

### BACKGROUND

Cell-free DNA (cfDNA) is a non-invasive biomarker that can inform on the diagnosis and prognosis of physiological and pathological conditions (1-3). cfDNA naturally exists as short DNA fragments typically <200 bp long (4).

Plasma DNA is believed to consist of cell-free DNA shed from multiple tissues in the body, including but not limited to, hematopoietic tissues, brain, liver, lung, colon, pancreas and so on (Sun et al, Proc Natl Acad Sci USA. 2015; 112:E5503-12; Lehmann-Werman et al, Proc Natl Acad Sci USA. 2016; 113: E1826-34; Moss et al, Nat Commun. 2018; 9: 5068). Plasma DNA molecules (a type of cell-free DNA molecules) have been demonstrated to be generated through a non-random process, for example, its size profile showing 166-bp major peaks and 10-bp periodicities occurring in the smaller peaks (Lo et al, Sci Transl Med. 2010; 2:61ra91; Jiang et al, Proc Natl Acad Sci USA. 2015; 112:E1317-25).

Recently, it was reported that a subset of human genomic locations (e.g., positions on a reference genome) are preferentially cut, thereby generating plasma DNA fragments having end positions that bear a relationship with the tissue of origin (Chan et al, Proc Natl Acad Sci USA. 2016; 113:E8159-8168; Jiang et al, Proc Natl Acad Sci USA. 2018; doi: 10.1073/pnas.1814616115). Chandrananda et al (BMC Med Genomics. 2015; 8: 29) used the de novo discovery software DREME (Bailey, Bioinformatics. 2011; 27:1653-9) to mine the cell-free DNA data for motifs related to nuclease cleavage, irrespective of tissue type.

### BRIEF SUMMARY

The present disclosure describes the scientific basis and practical implementation of using both ends of a cfDNA fragment as a biomarker, e.g., for cancer (or other pathology) detection, monitoring, and prognostication and for distinguishing different types of molecules (e.g., fetal/maternal molecules, tumor/normal molecules, or transplant/donor molecules). Some embodiments can be used for cancers including, but not limited to, hepatocellular carcinoma (HCC), colorectal cancer, lung cancer, nasopharyngeal cancer, head and neck squamous cell cancer, etc. Various embodiments can be used for distinguishing cfDNA fragments from fetal origin, a tumor, or donated tissue.

According to various embodiments, the present disclosure describes techniques for measuring quantities (e.g., relative frequencies) of end motif pairs of cell-free DNA fragments in a biological sample of an organism for measuring a property of the sample (e.g., fractional concentration of clinically-relevant DNA) and/or determining a pathology of the organism based on such measurements. Different tissue types exhibit different patterns for the relative frequencies of the end motif pairs. The present disclosure provides various uses for measurements of the relative frequencies of end motif pairs of cell-free DNA, e.g., in mixtures of cell-free DNA from various tissues. DNA from one of such tissue may be referred to as clinically-relevant DNA. In other examples, DNA from more than one such tissue may be referred to as clinically-relevant DNA.

Various examples can quantify amounts of end motif pairs representing the end sequences of DNA fragments. For example, embodiments can determine relative frequencies of a set of end motif pairs for ending sequences of DNA fragments. In various implementations, preferred sets of end motif pairs and/or patterns of end motif pairs can be determined using a genotypic (e.g., a tissue-specific allele) or a phenotypic approach (e.g., using samples that have a same pathology). The relative frequencies of a preferred set or having a particular pattern can be used to measure a classification of a property (e.g., fractional concentration of clinically-relevant DNA) of a new sample or a pathology (e.g., a level of cancer or disease in a particular tissue) of the organism. Accordingly, embodiments can provide measurements to inform physiological alterations, including cancers, autoimmune diseases, transplantation, and pregnancy.

As further examples, end motif pairs can be used in a physical enrichment and/or an in silico enrichment of a biological sample for cell-free DNA fragments that are clinically-relevant. The enrichment can use end motif pairs that are preferred for a clinically-relevant tissue, such as fetal, tumor, or transplant. The physical enrichment can use one or more probe molecules that detect a particular set of end motif pairs such that the biological sample is enriched for clinically-relevant DNA fragments. For the in silico enrichment, a group of sequence reads of cell-free DNA fragments having one of a set of preferred ending sequences for clinically-relevant DNA can be identified. Certain sequence reads can be stored based on a likelihood of corresponding to clinically-relevant DNA, where the likelihood accounts for the sequence reads including the preferred end motif pairs. The stored sequence reads can be analyzed to determine a property of the clinically-relevant DNA in the biological sample.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows examples for end motif pairs, including a single base at the ends of a DNA fragment, according to embodiments of the present disclosure.

FIG. 2 shows the construction of an A< >A fragment according to embodiments of the present disclosure.

FIG. 3 shows an analysis of sequencing data in a biological sample to determine end motif pairs according to an embodiment of the present invention.

FIGS. 4A-4C show different combinations for different categories of end motifs to categorize cfDNA fragments biterminally according to embodiments of the present disclosure.

FIGS. 5A-12D show classification results for all possible 1-mer biterminal fragment types according to embodiments of the present disclosure. The proportion for each 1-mer biterminal fragment is calculated in each sample and plotted in the corresponding boxplots. The ROC curve corresponding to the ability of the fragment type percentage in distinguishing between non-cancer (Control, HBV carrier (HBV), cirrhosis (cirr)) and cancer (early HCC (eHCC), intermediate HCC (iHCC), advanced HCC (aHCC)) is shown left of the boxplots with the AUC.

FIGS. 13A-18B show classification results for 2-mer biterminal fragments types that have an AUC>0.9 in distinguishing between non-cancer and HCC according to embodiments of the present disclosure.

FIGS. 19A-19D show the performance of a biterminal analysis with −1 and +1 position nucleotides in distinguishing HCC according to embodiments of the present disclosure.

FIGS. 20A-20C provide the performance of CG< >AA in distinguishing controls from HBV and cirrhosis according to embodiments of the present disclosure.

FIGS. 21A-21C provide the performance of GC< >TA in distinguishing controls from HBV and cirrhosis according to embodiments of the present disclosure. FIGS. 21D-21F provide the performance of TA< >GC in distinguishing controls from HBV and cirrhosis according to embodiments of the present disclosure.

FIGS. 22A-22C provide the performance of C< >C in distinguishing controls from HBV and cirrhosis according to embodiments of the present disclosure. FIGS. 22D-22F provide the performance of C< >A in distinguishing controls from HBV and cirrhosis according to embodiments of the present disclosure.

FIGS. 23-25B show ROC curves of CC< >CC fragment proportions and AUC values in distinguishing between controls and other cancers such as colorectal cancer (CRC), lung squamous cell carcinoma (LUSC), nasopharyngeal cancer (NPC), and head and neck squamous cell carcinoma (HNSCC) according to embodiments of the present disclosure.

FIGS. 26A-28B show the performance of three example biterminal fragments with −1 and +1 position nucleotides in distinguishing other cancers (CRC, LUSC, NPC, HNSCC) according to embodiments of the present disclosure.

FIGS. 29A-30B show the best performance for respective biterminal fragments with −1 and +1 position nucleotides in distinguishing each of CRC, LUSC, NPC, or HNSCC according to embodiments of the present disclosure.

FIG. 31 shows a table including performance results of the end motifs with the highest AUC in distinguishing among different stages of cancer according to embodiments of the present disclosure.

FIG. 32 shows a list 3200 of all 2end:−2+2 types with 100% accuracy for distinguishing between intermediate and advanced HCC and a list 3250 of all 2end:−2+2 types with 100% accuracy for distinguishing between early and advanced HCC according to embodiments of the present disclosure.

FIGS. 33A-33D provide performance results for the best performing biterminal −1 and +1 position motifs in distinguishing early vs intermediate HCC according to embodiments of the present disclosure.

FIGS. 34A-34D provide performance results for the best performing biterminal −1 and +1 position motifs in distinguishing intermediate vs advanced HCC according to embodiments of the present disclosure.

FIGS. 35A-35D provide performance results for the best performing biterminal −1 and +1 position motifs in distinguishing early vs advanced HCC according to embodiments of the present disclosure.

FIGS. 36A-36D provide performance results for the best performing biterminal −1 and +1 position motifs in distinguishing early vs advanced HCC according to embodiments of the present disclosure.

FIGS. 37A-37D show performance for C< >C in distinguishing controls, inactive SLE, and active SLE according to embodiments of the present disclosure.

FIGS. 38A-38D show performance for A< >A in distinguishing controls, inactive SLE, and active SLE according to embodiments of the present disclosure.

FIGS. 39A-39D show performance for GT< >TG in distinguishing controls, inactive SLE, and active SLE according to embodiments of the present disclosure.

FIGS. 40A-40D show performance for TG< >CC in distinguishing controls, inactive SLE, and active SLE according to embodiments of the present disclosure.

FIGS. 41A-41D show performance for TG< >GG in distinguishing controls, inactive SLE, and active SLE according to embodiments of the present disclosure.

FIGS. 42A-42D show performance for c|A< >a|A in distinguishing controls, inactive SLE, and active SLE according to embodiments of the present disclosure.

FIGS. 43A-43D show performance for g|C< >g|C in distinguishing controls, inactive SLE, and active SLE according to embodiments of the present disclosure.

FIGS. 44A-44B show the performance for C< >C fragments in distinguishing between non-cancer and HCC using fewer fragments (20 million fragments) in each sample according to embodiments of the present disclosure.

FIG. 45 is a graph depicting the AUC achievable using CC< >CC fragments as a function of the total number of fragments sequenced estimated through a downsampling analysis according to embodiments of the present disclosure.

FIG. 46 is a flowchart illustrating a method for determining a level of pathology using end motif pairs of cell-free DNA fragments according to embodiments of the present disclosure.

FIG. 47 shows multiple ROC curves from different methods of analysis on the same non-HCC and HCC dataset according to embodiments of the present disclosure.

FIGS. 48-50B show multiple ROC curves from different methods of analysis of a data set with 30 controls and 40 other cancers with CRC, LUSC, NPC, and HNSCC according to embodiments of the present disclosure.

FIGS. 51A-51B show a biterminal analysis in differentiating between fetal-specific molecules and shared molecules according to embodiments of the present disclosure.

FIG. 52A shows a functional relationship between biterminal C< >C % and the fetal DNA fraction according to embodiments of the present disclosure. FIG. 52B shows a functional relationship between biterminal CC< >CC % and the fetal DNA fraction according to embodiments of the present disclosure.

FIG. 53 shows the functional relationship between C< >G % and tumor concentration according to embodiments of the present disclosure.

FIGS. 54A-55B show a biterminal analysis in differentiating between done-specific molecules and shared molecules for a liver transplant subject according to embodiments of the present disclosure.

FIGS. 56A-56B show a biterminal analysis in differentiating between done-specific molecules and shared molecules for a kidney transplant subject according to embodiments of the present disclosure.

FIG. 57 is a flowchart illustrating a method of estimating a fractional concentration of clinically-relevant DNA in a biological sample of a subject according to embodiments of the present disclosure.

FIG. 58 shows an ROC curve for SVM modeling using end motif pairs of −1 and +1 position nucleotides to distinguish non-cancer and HCC subjects according to embodiments of the present disclosure.

FIG. 59 is a flowchart illustrating a method of physically enriching a biological sample for clinically-relevant DNA according to embodiments of the present disclosure.

FIG. 60 is a flowchart illustrating a method for in silico enriching of a biological sample for clinically-relevant DNA according to embodiments of the present disclosure.

FIG. 61 illustrates a measurement system according to an embodiment of the present invention.

FIG. 62 shows a block diagram of an example computer system usable with systems and methods according to embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells. Multiple samples of a same tissue type from different individuals may be used to determine a tissue-specific methylation level for that tissue type.

A “biological sample” refers to any sample that is taken from a subject (e.g., a human (or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest. The biological sample can be a bodily fluid, such as blood, plasma, serum, urine, vaginal fluid, fluid from a hydrocele (e.g. of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g. thyroid, breast), intraocular fluids (e.g. the aqueous humor), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at for example, 30,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

A “sequence read” refers to a string of nucleotides sequenced from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. As part of an analysis of a biological sample, a statistically significant number of sequence reads can be analyzed, e.g., at least 1,000 sequence reads can be analyzed. As other examples, at least 10,000 or 50,000 or 100,000 or 500,000 or 1,000,000 or 5,000,000 sequence reads, or more, can be analyzed.

A “cutting site” can refer to a location that DNA was cut by a nuclease, thereby resulting in a DNA fragment.

A sequence read can include an “ending sequence” associated with an end of a fragment. The ending sequence can correspond to the outermost N bases of the fragment, e.g., 1-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” may refer to a short, recurring pattern of bases in DNA fragments (e.g., cell-free DNA fragments). A sequence motif can occur at an end of a fragment, and thus be part of or include an ending sequence. An “end motif” can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif.

A “sequence motif pair” or “end motif pair” may refer to a pair of end motifs of a particular DNA fragment. For example, a DNA fragment having an A at the 5′ end of one strand and an A at the 5′ end of the other strand can be defined as having a sequence motif pair of A< >A. As another example, a DNA fragment having an A at the 5′ end of one strand and an T at the 3′ end of the same strand can be defined as having a sequence motif pair of A< >T, which would correspond to an A< >A fragment defined using the 5′ ends of the two strands. Other lengths of sequence motifs can be used. Different paired combinations of end motifs can be referred to as different types of fragments. End motif pairs may include end motifs that are the same length, e.g., both 1-mers or both 2-mers, but may also include end motifs that are of different lengths, e.g., one end is a 2-mer and the other end is composed of 1-mers. End motif pairs may also include one or more bases past the end of the DNA fragment, e.g., as determined by aligning to a reference genome. Such an instance can use the nomenclature t|A, where T occurs just before a cutting site at the 5′ end, and A occurs after the cutting site.

The term “alleles” refers to alternative DNA sequences at the same physical genomic locus, which may or may not result in different phenotypic traits. In any particular diploid organism, with two copies of each chromosome (except the sex chromosomes in a male human subject), the genotype for each gene comprises the pair of alleles present at that locus, which are the same in homozygotes and different in heterozygotes. A population or species of organisms typically include multiple alleles at each locus among various individuals. A genomic locus where more than one allele is found in the population is termed a polymorphic site. Allelic variation at a locus is measurable as the number of alleles (i.e., the degree of polymorphism) present, or the proportion of heterozygotes (i.e., the heterozygosity rate) in the population. As used herein, the term “polymorphism” refers to any inter-individual variation in the human genome, regardless of its frequency. Examples of such variations include, but are not limited to, single nucleotide polymorphism, simple tandem repeat polymorphisms, insertion-deletion polymorphisms, mutations (which may be disease causing) and copy number variations. The term “haplotype” as used herein refers to a combination of alleles at multiple loci that are transmitted together on the same chromosome or chromosomal region. A haplotype may refer to as few as one pair of loci or to a chromosomal region, or to an entire chromosome or chromosome arm.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672). Similarly, tumor fraction or tumor DNA fraction can refer to the fractional concentration of tumor DNA in a biological sample.

A “relative frequency” (also referred to just as “frequency”) may refer to a proportion (e.g., a percentage, fraction, or concentration). In particular, a relative frequency of a particular end motif pair (e.g., A< >A) can provide a proportion of cell-free DNA fragments that have that particular pair of ending sequences.

An “aggregate value” may refer to a collective property, e.g., of relative frequencies of a set of end motifs. Examples include a mean, a median, a sum of relative frequencies, a variation among the relative frequencies (e.g., entropy, standard deviation (SD), the coefficient of variation (CV), interquartile range (IQR) or a certain percentile cutoff (e.g. 95^{th} or 99^{th} percentile) among different relative frequencies), or a difference (e.g., a distance) from a reference pattern of relative frequencies, as may be implemented in clustering. As another example, an aggregate value can comprise an array/vector of relative frequencies, which can be compared to a reference vector (e.g., representing a multidimensional data point).

The term “sequencing depth” refers to the number of times a locus is covered by a sequence read aligned to the locus. The locus could be as small as a nucleotide, or as large as a chromosome arm, or as large as the entire genome. Sequencing depth can be expressed as 50×, 100×, etc., where “x” refers to the number of times a locus is covered with a sequence read. Sequencing depth can also be applied to multiple loci, or the whole genome, in which case x can refer to the mean number of times the loci or the haploid genome, or the whole genome, respectively, is sequenced. Ultra-deep sequencing can refer to at least 100× in sequencing depth.

A “calibration sample” can correspond to a biological sample whose fractional concentration of clinically-relevant DNA (e.g., tissue-specific DNA fraction) is known or determined via a calibration method, e.g., using an allele specific to the tissue, such as in transplantation whereby an allele present in the donor's genome but absent in the recipient's genome can be used as a marker for the transplanted organ. As another example, a calibration sample can correspond to a sample from which end motifs can be determined. A calibration sample can be used for both purposes.

A “calibration data point” includes a “calibration value” and a measured or known fractional concentration of the clinically-relevant DNA (e.g., DNA of particular tissue type). The calibration value can be determined from relative frequencies (e.g., an aggregate value) as determined for a calibration sample, for which the fractional concentration of the clinically-relevant DNA is known. The calibration data points may be defined in a variety of ways, e.g., as discrete points or as a calibration function (also called a calibration curve or calibration surface). The calibration function could be derived from additional mathematical transformation of the calibration data points.

A “separation value” corresponds to a difference or a ratio involving two values, e.g., two fractional contributions or two methylation levels. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (ln) of the two values. A separation value can include a difference and a ratio.

A “separation value” and an “aggregate value” (e.g., of relative frequencies) are two examples of a parameter (also called a metric) that provides a measure of a sample that varies between different classifications (states), and thus can be used to determine different classifications. An aggregate value can be a separation value, e.g., when a difference is taken between a set of relative frequencies of a sample and a reference set of relative frequencies, as may be done in clustering.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1).

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter.

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g. recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g. symptoms or other positive tests), has cancer.

A “level of pathology” can refer to the amount, degree, or severity of pathology associated with an organism, where the level can be as described above for cancer. Another example of pathology is a rejection of a transplanted organ. Other example pathologies can include autoimmune attack (e.g., lupus nephritis damaging the kidney or multiple sclerosis damaging the central nervous system), inflammatory diseases (e.g., hepatitis), fibrotic processes (e.g. cirrhosis), fatty infiltration (e.g. fatty liver diseases), degenerative processes (e.g. Alzheimer's disease) and ischemic tissue damage (e.g., myocardial infarction or stroke). A heathy state of a subject can be considered a classification of no pathology.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

The present disclosure describes techniques for measuring quantities (e.g., relative frequencies) of end motif pairs of cell-free DNA fragments in a biological sample of an organism for measuring a property of the sample and/or determining a pathology of the organism based on such measurements. Different tissue types exhibit different patterns for the relative frequencies of the end motif pairs. The present disclosure provides various uses for measurements of the relative frequencies of end motif pairs of cell-free DNA, e.g., in mixtures of cell-free DNA from various tissues. DNA from one of such tissues may be referred to as clinically-relevant DNA.

As an example of a pathology, a level of cancer can be determined using relative frequencies of end motif pairs among the cell-free DNA fragments of a sample. An organism having different phenotypes can exhibit different patterns of relative frequencies of the end motif pairs of cell-free DNA fragments. An aggregate value of relative frequencies of end motif pairs can be compared to a reference value to classify the phenotype. In various implementations, the aggregate value can be a sum of relative frequencies or a difference from a reference set of relative frequencies.

As another example, clinically-relevant DNA of a particular tissue (e.g., of a fetus, a tumor, or a transplanted organ) exhibit a particular pattern of relative frequencies, which can be measured as an aggregate value. Other DNA in a sample can exhibit a different pattern, thereby allowing a measurement of an amount of clinically-relevant DNA in the sample. Accordingly, in one example, a fractional concentration (e.g., a percentage) of clinically relevant DNA can be determined based on relative frequencies of end motif pairs. The fractional concentration can be a number, a numerical range, or other classification, e.g., high, medium, or low, or whether the fractional concentration exceeds a threshold. In various implementations, the aggregate value could be a sum of relative frequencies for a set of end motif pairs or a difference (e.g., total distance) from a reference pattern, e.g., an array (vector) of relative frequencies for calibration sample(s) with a known fractional concentration. Such an array can be considered a reference set of relative frequencies. Such a difference can be used in a classifier of which hierarchal clustering, support vector machines, and logistic regression are examples. As examples, the clinically relevant DNA can be fetal, tumor, transplanted organ, or other tissue (e.g. hematopoietic or liver) DNA.

Given that cell-free DNA fragments having a particular set of end motif pairs are differentially represented (quantified by relative frequency) in a certain tissue compared to other tissue (e.g., fetal vs. maternal), these end motif pair(s) can be used to enrich a sample for DNA from the certain tissue (clinically-relevant DNA). Such enrichment can be performed via physical operations to enrich the physical sample. Some embodiments can capture and/or amplify cell-free DNA fragments having ending sequences matching a set of preferred end motif pairs, e.g., using primers or adapters. Other examples are described herein. When the representation in relative frequency is higher in the clinically-relevant DNA for a set of end motif pair(s), then one can refer to those as preferred end motif pair(s).

In some embodiments, the enrichment can be performed in silico. For example, a system can receive sequence reads and then filter the reads based on end motif pairs to obtain a subset of sequence reads that have a higher concentration of corresponding DNA fragments from the clinically-relevant DNA. If a DNA fragment has ending sequences that are a preferred end motif pair, that DNA fragment can be identified as having a higher likelihood of being from the tissue of interest. The likelihood can be further determined based on methylation and size of the DNA fragments, as is described herein.

Such uses of end motif pairs can obviate a need for a reference genome, as may be needed when using end positions (Chan et al, Proc Natl Acad Sci USA. 2016; 113:E8159-8168; Jiang et al, Proc Natl Acad Sci USA. 2018; doi: 10.1073/pnas.1814616115). Further, as the number of end motif pairs may be smaller than the number of preferred end positions in a reference genome, greater statistics can be gathered for each end motif pair, potentially increasing accuracy.

Such an ability to use end motif pairs in the manner described above is surprising, e.g., as Chandrananda et al. found that there was high similarity between maternal and fetal fragments in terms of position-specific nucleotide patterns concerning mononucleotide frequencies for the region of 51 bp (up-/down-stream 20 bp) around fragment start sites (Chandrananda et al, BMC Med Genomics. 2015; 8:29), implying that the use of their method based on mononucleotide frequencies around ends was unable to inform the tissue of origin of the cell-free DNA fragments.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### I. Cell-Free DNA End Motif Pairs (Biterminal Analysis)

An end motif relates to the ending sequence of a cell-free DNA fragment, e.g., the sequence for the K bases at either end of the fragment. On the other hand, an end motif pair relates to both the ending sequences of a fragment. The ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments.

A. Example Determination of End Motif Pairs

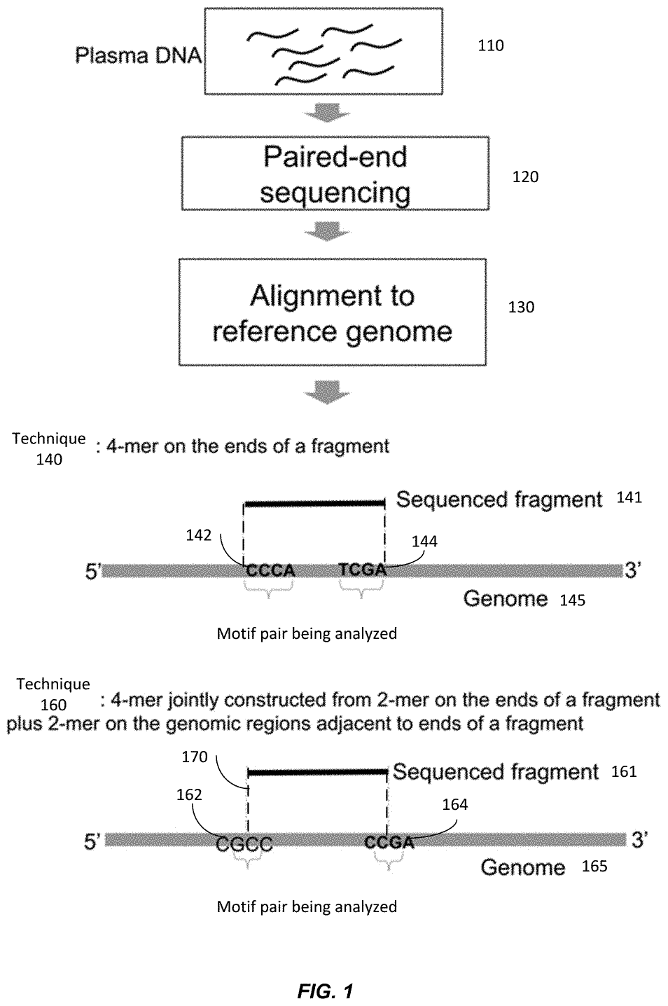

FIG. 1 shows examples for end motif pairs according to embodiments of the present disclosure. FIG. 1 depicts two ways to define 4-mer end motifs to be analyzed. In technique 140, the 4-mer end motifs are directly constructed from the first 4-bp sequence on each end of a plasma DNA molecule. For example, the first 4 nucleotides and the last 4 nucleotides of a sequenced fragment could be used as an end motif pair. In technique 160, the 4-mer end motifs are jointly constructed by making use of the 2-mer sequence from the sequenced ends of fragments and the other 2-mer sequence from the genomic regions adjacent to the ends of that fragment. In other embodiments, other types of motifs can be used, e.g., 1-mer, 2-mer, 3-mer, 5-mer, 6-mer, 7-mer end motifs.

As shown in FIG. 1, cell-free DNA fragments 110 are obtained, e.g., using a purification process on a blood sample, such as by centrifuging. Besides plasma DNA fragments, other types of cell-free DNA molecules can be used, e.g., from serum, urine, saliva, or other bodily fluids. The DNA fragments may be blunt-ended.

At block 120, the DNA fragments are subjected to paired-end sequencing. In some embodiments, the paired-end sequencing can produce two sequence reads from the two ends of a DNA fragment, e.g., 30-120 bases per sequence read. These two sequence reads can form a pair of reads for the DNA fragment (molecule), where each sequence read includes an ending sequence of a respective end of the DNA fragment. In other embodiments, the entire DNA fragment can be sequenced, thereby providing a single sequence read, which includes the ending sequences of both ends of the DNA fragment. The two ending sequences at both ends can still be considered paired sequence reads, even if generated together from a single sequencing operation.

At block 130, the sequence reads can be aligned to a reference genome. This alignment is to illustrate different ways to define a sequence motif, and may not be used in some embodiments. For example, the sequences at the end of a fragment can be used directly without needing to align to a reference genome. However, alignment can be desired to have uniformity of an ending sequence, which does not depend on variations (e.g., SNPs) in the subject. For instance, the ending base could be different from the reference genome due to a variation or a sequencing error, but the base in the reference may be the one counted. Alternatively, the base on the end of the sequence read can be used, so as to be tailored to the individual. The alignment procedure can be performed using various software packages, such as (but not limited to) BLAST, FASTA, Bowtie, BWA, BFAST, SHRiMP, SSAHA2, NovoAlign, and SOAP.

Technique 140 shows a sequence read of a sequenced fragment 141, with an alignment to a reference genome 145. With the 5′ end viewed as the start, a first end motif 142 (CCCA) is at the start of sequenced fragment 141. A second end motif 144 (TCGA) is at the tail of the sequenced fragment 141. When analyzing the end predominance of cfDNA fragments, this sequence read would contribute to a count for C-end for the 5′ end and an A-end for the 3′ end (or a T-end if the 5′ end of the other strand is used). Such end motifs might, in one embodiment, occur when an enzyme recognizes CCCA and then makes a cut just before the first C. If that is the case, CCCA will preferentially be at the end of the plasma DNA fragment. For TCGA, an enzyme might recognize it, and then make a cut after the A. Such an end motif pair can be labeled as CCCA< >TCGA, depending on the convention used. Various examples of different conventions are provided below. For instance, a convention for the second end motif can be read on from the 5′ end of the other strand. With TCGA, the complement is the same; but if the 3′ end sequence was TTGA, then the 5′ convention would be TCAA as the sequence starts at the end. This 5′ convention for both ends is used in the examples. When a 1-mer count is determined for end motif pairs, this sequence read would contribute to a C< >T count using the 5′ convention. Using technique 140, alignment to a reference genome can be optional.

Technique 160 shows a sequence read of a sequenced fragment 161, with an alignment to a reference genome 165. With the 5′ end viewed as the start, a first end motif 162 (CGCC) has a first portion (CG) that occurs just before the start of sequenced fragment 161 and a second portion (CC) that is part of the ending sequence for the start of sequenced fragment 161. A second end motif 164 (CCGA) has a first portion (GA) that occurs just after the tail of sequenced fragment 161 and a second portion (CC) that is part of the ending sequence for the tail of sequenced fragment 161. Such end motifs might, in one embodiment, occur when an enzyme makes a cut after the G, just before the C. If that is the case, CC will preferentially be at the end of the plasma DNA fragment with CG occurring just before it, thereby providing an end motif of CGCC. As for the second end motif 164 (CCGA), an enzyme can cut between C and G. If that is the case, CC will preferentially be at the 3′ end of the plasma DNA fragment. Such an end motif pair can be labeled as cg|CC< >tc|GG, where TCGG is the CCGA motif from the 5′ end of the reverse strand and the lowercase letters signify that the bases are on the other side of the cutting site 170, which is signified by the dotted line. The cutting site is where an enzyme (e.g., a nuclease) cuts the sequenced fragment 161. For technique 160, the number of bases from the adjacent genome regions and sequenced plasma DNA fragments can be varied and are not necessarily restricted to a fixed ratio, e.g., instead of 2:2, the ratio can be 2:3, 3:2, 4:4, 2:4, etc.

The higher the number of nucleotides included in the cell-free DNA end pair signature, the higher the specificity of the motif because the probability of having 6 bases ordered in an exact configuration in the genome at two locations (˜50-30 bp apart) is lower than the probability of having 2 bases ordered in an exact configuration at two locations in the genome. Thus, the choice of the length of the end motif can be governed by the needed sensitivity and/or specificity of the intended use application.

When the ending sequence is used to align the sequence read to the reference genome (e.g. in technique 160), any sequence motif determined from the ending sequence or just before/after is still determined from the ending sequence. Thus, technique 160 makes an association of an ending sequence to other bases, where the reference is used as a mechanism to make that association. A difference between techniques 140 and 160 would be to which two end motifs a particular DNA fragment is assigned, which affects the particular values for the relative frequencies. But, the overall result (e.g., determining a classification or a pathology, determining a fractional concentration of clinically-relevant DNA, etc.) would not be affected by how a DNA fragment is assigned to an end motif pair, as long as a consistent technique is used, e.g., for any training data to determine a reference value, as may occur using a machine learning model.

The counted numbers of DNA fragments having ending sequences corresponding to a particular end motif pair may be counted (e.g., stored in an array in memory) to determine an amount of the particular end motif pair. The amount can be measured in various ways, such as a raw count or a frequency, where the amount is normalized. The normalization may be done using (e.g., dividing by) a total number of DNA fragments or a number in a specified group of DNA fragments (e.g., from a specified region, having a specified size, or having one or more specified end motifs). Differences in amounts of end motif pairs have been detected when cancer exists and when a sample includes different fractional concentrations of clinically-relevant DNA.

B. End Motif Pairs Defined on Watson and Crick Strands

An end motif pair can be defined in various ways, some of which are mentioned above. In some embodiments, an end motif pair are defined using both the Watson strand and the Crick strand. In this manner, the sequences at the 5′ ends are used.

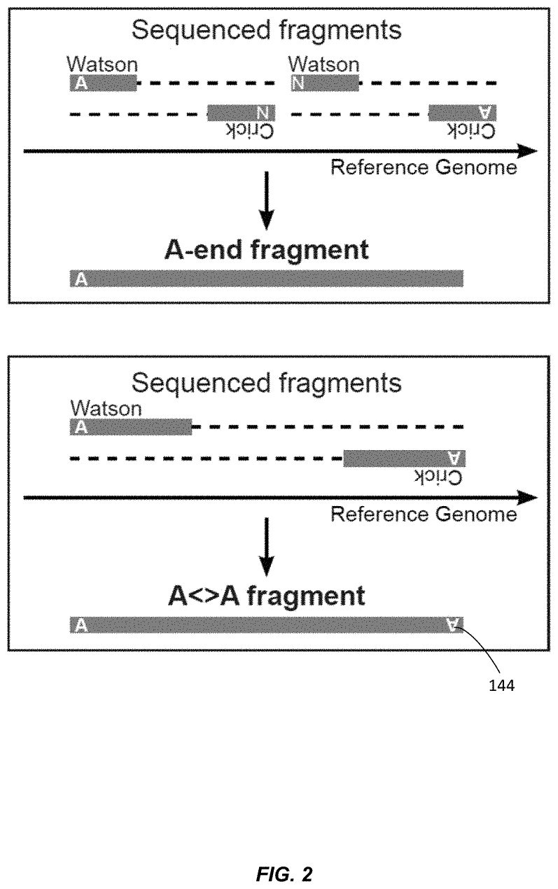

FIG. 2 shows the construction of an A< >A fragment according to embodiments of the present disclosure. FIG. 2 shows an A-end fragment and an A< >A fragment. An A-end fragment has an A at the 5′ end of the Watson strand or at the 5′ end of the Crick strand. The other end can be signified with N, since the base could be any base. An A< >A fragment has an A at the 5′ end of the Watson strand and an A at the 5′ end of the Crick strand. Such nomenclature also applies to C< >C, G< >G, and T< >T, all of which are used throughout the disclosure.

Such a nomenclature corresponding to the two strands can still be used when sequencing is performed on single strands of DNA. For example, the end sequence at the 3′ end of one strand (e.g., the Watson strand) can be converted to the complementary end sequence at the 5′ end of the other strand. Thus, the end sequence can, by convention, be the complementary sequence to the base at the 3′ end. Such single strand sequencing may occur in bisulfite sequencing. To distinguish between A< >C or C< >A when single strand sequencing is done, one may or may not align to a reference genome. But since such symmetrical fragment types typically have the same behavior, there may be no need to distinguish and they can be counted together as a single group.

C. Sequencing and Alignment for Watson/Crick Strands

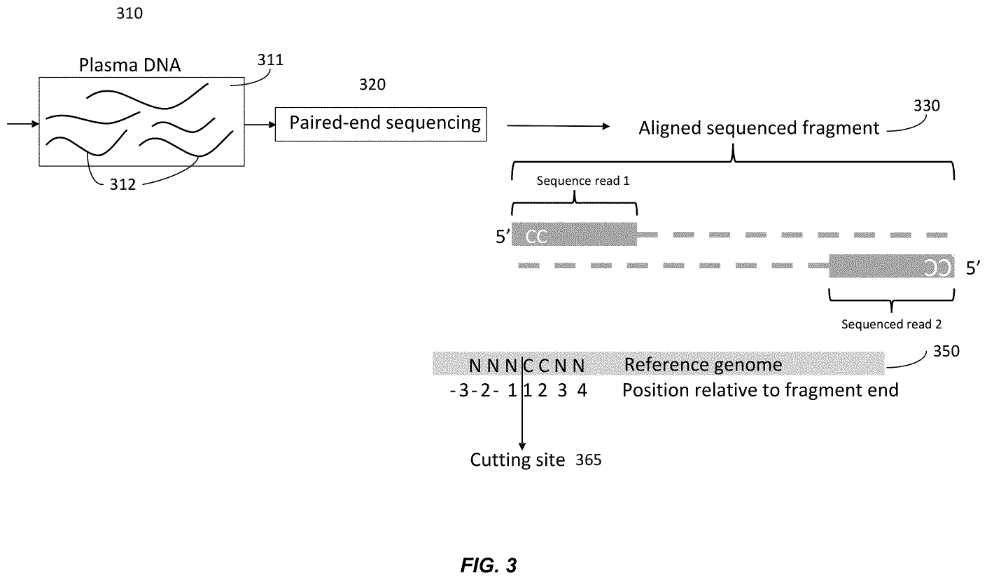

FIG. 3 shows an analysis of sequencing data in a biological sample to determine end motif pairs according to an embodiment of the present invention. The biological sample may be obtained from a person suspected of having cancer (e.g., hepatocellular carcinoma (HCC)). Although HCC is used as an example, embodiments are applicable to other cancers.

In step 310, a biological sample 311 from a patient suspected of having HCC is received. The biological sample may be from any bodily fluid including but not limited to plasma, serum, urine, and saliva. The sample contains cell-free nucleic acid molecules 312. In one embodiment, DNA is extracted from the plasma of a patient.

In step 320, a sequencing library is constructed from the plasma DNA using, for example, but not limited to, the Illumina TruSeq Nano kit. Other sequencing library preparation kits can also be used. At least a portion of a plurality of the nucleic acid molecules contained in the biological sample are sequenced. The sequenced portion may represent a fraction of the human genome, an entirety of the human genome (or other genome for other animals, plants, etc.), or be at multiple folds of sequencing depth. Both ends of varying lengths or the entire fragment may be sequenced. All or just a subset of the nucleic acid molecules in the sample may be sequenced. This subset may be chosen randomly or in a targeted method, e.g., using probes to capture certain sequences (e.g., corresponding to one or more particular loci/regions) or using primers to amplify certain sequences. In one embodiment, the sequencing is done using paired-end massively parallel sequencing, e.g., with the Illumina HiSeq 4000 platform. Other sequencing platforms may be used.

Based on the sequencing data of a fragment, the nucleotides at the fragment ends are determined. A bioinformatics procedure may be used to discard a proportion of sequenced data from subsequent analysis because they are of poor quality or deemed to be PCR duplicates. In one embodiment with paired-end sequencing, the 5′ end of read 1 and the 5′ end of read 2 represent the ends of a fragment. If a full molecule is sequenced, then both ends can be determined from one read.

In step 330, the sequenced data may be aligned (mapped) to the reference human genome 350, e.g., to determine the size of a fragment. For instance, read 1 and read 2 can be aligned together as a pair. With alignment, nucleotide information at the −1, −2, −3, −4 positions may also be obtained. Fragment size information may also be obtained. As another example, a size may be obtained without resorting to alignment, e.g., when the entire DNA molecule is sequenced.

Fragments can be categorized and counted based on the nucleotides at both ends. In one embodiment, only one nucleotide on each end is used to categorize fragments into 16 types. More nucleotides, for example, 2-mer, 3-mer etc., can be used within the fragment to categorize fragments. The nucleotide sequences on the other side of the cleavage position (cutting site) 365, for example at position −1, −2, −3, −4 etc., can also be used to categorize fragments. As shown, the reference genome 350 has N listed at these positions, as the CC ends are highlighted. In practice, the actual bases can be obtained after alignment.

In some embodiments, rules may be imposed on the sequencing data to determine what gets counted. For example, sequencing data corresponding to nucleic acid fragments of a specified size range could be selected after bioinformatics analysis. Examples of size ranges are <150 bp, 150-250 bp, >250 bp.

The fragment type amounts may be simply counted or a parameter can be determined from the categories of fragments. The parameter may be, for example, a simple ratio of a first amount of a certain fragment type (e.g., number of fragments with the particular end motif pair(s)) and a total amount of fragments. The parameter may include more than one fragment type in the first amount.

The parameter can be compared to one or more cutoff values to distinguish between different classifications of a condition. The cutoff values may be determined in any number of suitable ways from a training set of samples having a known classification (e.g., healthy or diseased). For instance, the parameter (e.g., the fractional representation of a fragment type) can be compared to a reference range (example of a cutoff) established in normal subjects. Based on the comparison, a classification of whether or not the patient is likely to have a condition (e.g., cancer) is determined.

D. Combinations of End Motif Pairs

The number of possible fragment types will depend on the number of bases used in the two end motifs. If the total number of bases used is M, then the total number of combinations is M⁴. For instance, if a 1-mer is used on both ends, then M is 2, and the total number of combination is 2⁴=16 different combinations. If a 2-mer is used on both ends, then M is 4, and the total number of combination is 4⁴=256 different combinations. If a 1-mer is used on one end and a 2-mer is used on another end, then M is 3, and the total number of combination is 3⁴=81 different combinations.

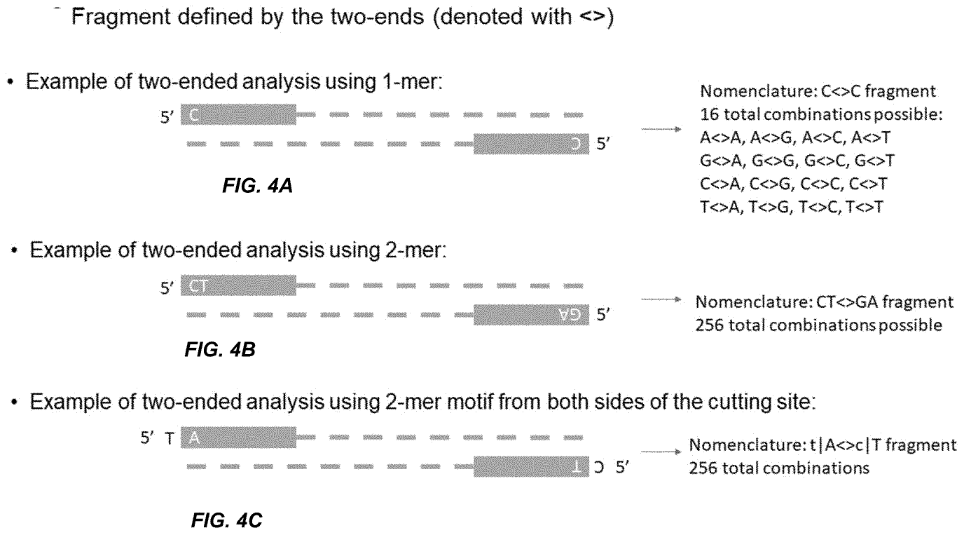

FIGS. 4A-4C show different combinations for different categories of end motifs to categorize cfDNA fragments biterminally according to embodiments of the present disclosure. FIG. 4A shows the 16 different fragment types when a 1-mer is used at both ends. The nomenclature of A< >A, A< >G, C< >C (example shown), etc. is used in FIG. 4A and throughout this disclosure. As shown, the 1-mers are determined at the 5′ ends of both fragments, but other conventions are possible, as is described herein.

FIG. 4B illustrates the use of 2-mers at both ends on the fragments, resulting in 256 different fragment types. The example fragment has end motifs CT and GA, which can be labeled as CT< >GA.

FIG. 4C illustrates the use of 2-mer motifs, with one base on the fragment and another base off the fragment (i.e., on the other side of the cutting site). The use of 2-mers for the end motif pairs still results in 256 different fragment types. But the nomenclature is different, given the use of a base off of the fragments; such a base can be determined by alignment to the reference genome. The example fragment has end motifs TA (with T off of the fragment) and CT (with C off of the fragment). In this disclosure, the nomenclature for the example fragment is t|A< >c|T.

Accordingly, the sequences at both ends of a fragment can be used to define a fragment type. The analysis can be performed with 1-mer, 2-mer, 3-mer etc. at variable positions around the fragment cutting site. Fragment ends may be defined only by the nucleotides at the −1, −2, −3 etc. positions as well (i.e. from the other side of the cutting site). The motifs analyzed around a cutting site need not be symmetrical, e.g., there may be one nucleotide before the cut and two nucleotides after the cut, and the nucleotides can be different before and after the cut. Sequences at fragment ends may be determined by sequencing technology or by probe/primer-based (e.g., PCR-based) methods. Examples of using PCR-based methods may include, but are not limited to, designing primers/probes for motifs that are commonly cut e.g., ct|CCCA and detecting quantitative changes. As another example, ligase chain reaction may be used where ligation and subsequent amplification only occurs when there is perfect complementarity between two probes. Probes can be designed to be complementary to the end motif sequences.

### II. Screening for Liver Pathologies

Different fragment types for cell-free DNA may occur in different amounts in plasma and other cell-free samples for different cohorts of subjects. In this section, we show that different fragments types can be used to screen for different liver pathologies, such as cancer (e.g., HCC), HBV, or cirrhosis. The ability to discriminate between subjects with HCC and without HCC is shown using 1-mers and 2-mers for the end motifs, as well as the ability to discriminate between early, intermediate, and advances stages of HCC.

To test the potential of biterminal analysis, we used a dataset containing 20 healthy control subjects (Control), 22 chronic hepatitis B carriers (HBV), 12 cirrhosis subjects (Cirr), 24 early-stage HCC (eHCC), 11 immediate-stage HCC (iHCC), and 7 advanced-stage HCC (aHCC) with a median number of paired-reads of 215 million (range: 97-1,681 million). This amount of sequencing roughly corresponds to a sequencing depth of 10-100×. Accordingly, plasma samples from 6 different cohorts of subjects were used, with potentially four levels of cancer, include no cancer and the three cancer stages. And a total of 96 subjects were used. In this section, all 16 types of 1-mer end motif pairs were analyzed. We used Illumina-based sequencing, although other sequencing platforms may be used. Bisulfite sequencing was used, but other sequencing (e.g., DNA of non-bisulfate treated DNA, i.e., DNA-seq) can be used as well. The classification of the cancer is based on the Barcelona Clinic Liver Cancer Staging system, which is based on a number of clinical parameters.

A. 1-Mer End Motif Pairs in HCC

In this biterminal analysis using only 1-mers, fragments were defined by the 1-mer end nucleotide on each end of the fragment, as opposed to using a 1-mer on the other side of the cutting site. The proportion (example of a relative frequency) of each fragment type (particular end motif pair) was calculated in each sample. For example, the proportion of C< >C fragments (C< >C %) was calculated as the number of C< >C fragment/the total number of all types of fragments.

Using this fragment type proportion, we analyzed the area under the curve (AUC) of the receiver operating characteristic (ROC) curve and its potential to distinguish the non-cancer samples (control, HBV, Cirr) and the cancer samples (eHCC, iHCC, aHCC) in each of the 16 types of fragments possible using 1-mer biterminal ends.

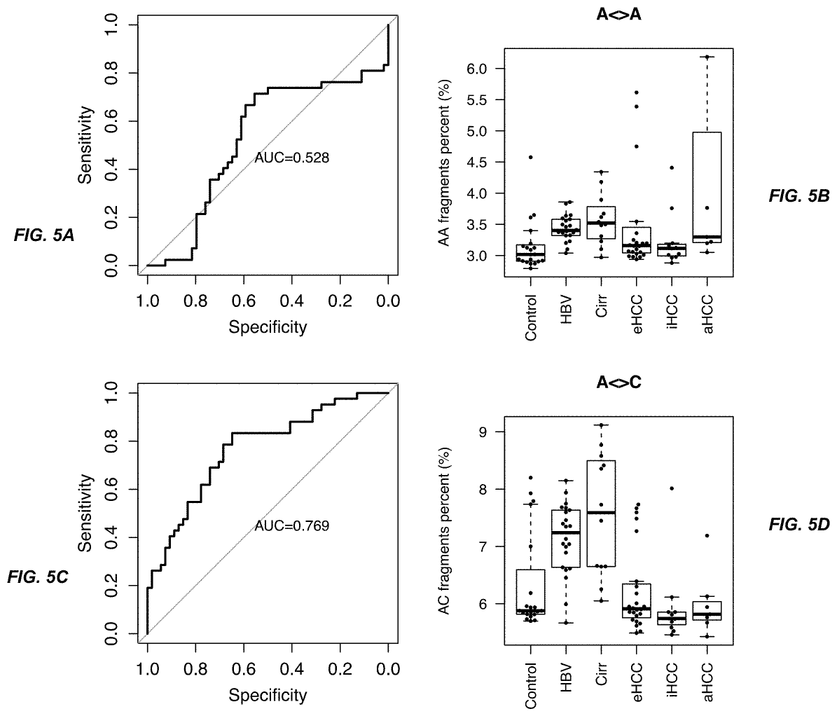

FIGS. 5A-12D show classification results for all possible 1-mer biterminal fragment types according to embodiments of the present disclosure. The proportion for each 1-mer biterminal fragment is calculated in each sample and plotted in the corresponding boxplots for each of the six cohorts of subjects. The ROC curve corresponding to the ability of the fragment type percentage in distinguishing between non-cancer (Control, HBV carrier (HBV), cirrhosis (cirr)) and cancer (early HCC (eHCC), intermediate HCC (iHCC), advanced HCC (aHCC)) is shown left of the boxplots with the AUC. Of the 16 types, C< >C % performed best with an AUC=0.91.

1. Results for A

FIGS. 5A-5B show classification results for the 96 subjects using A< >A fragments according to embodiments of the present disclosure. FIG. 5A shows a receiver operating characteristic (ROC) curve for the A< >A fragments. FIG. 5B shows a box plot of the percent of A< >A fragments for the six types of subjects. As one can see in FIG. 5B, the difference between the 3 non-cancer cohorts and the 3 cancer cohorts is not significant, resulting in a small AUC in FIG. 5A.

FIGS. 5C-5D show classification results for the 96 subjects using A< >C fragments according to embodiments of the present disclosure. FIG. 5C shows an ROC curve for the A< >C fragments. FIG. 5D shows a box plot of the percent of A< >C fragments for the six types of subjects. Different from FIG. 5B, the non-cancer subjects generally have a higher A< >C proportion that than the cancer subjects. This difference results in a better AUC in the ROC curve. As shown in FIG. 5D, a parameter of the proportion of DNA fragments having A< >C ends can provide a sensitivity of ˜0.8 and specificity of about ˜0.65 with a suitable choice of a reference value that discriminates between the cancer and non-cancer subjects. Higher or lower references values can result in a tradeoff between an increasing/decreasing of the sensitivity and specificity. The skilled person will appreciate the tradeoffs between sensitivity and specificity and be able to select a suitable reference (cutoff) value for any set of one or more end motif pairs.

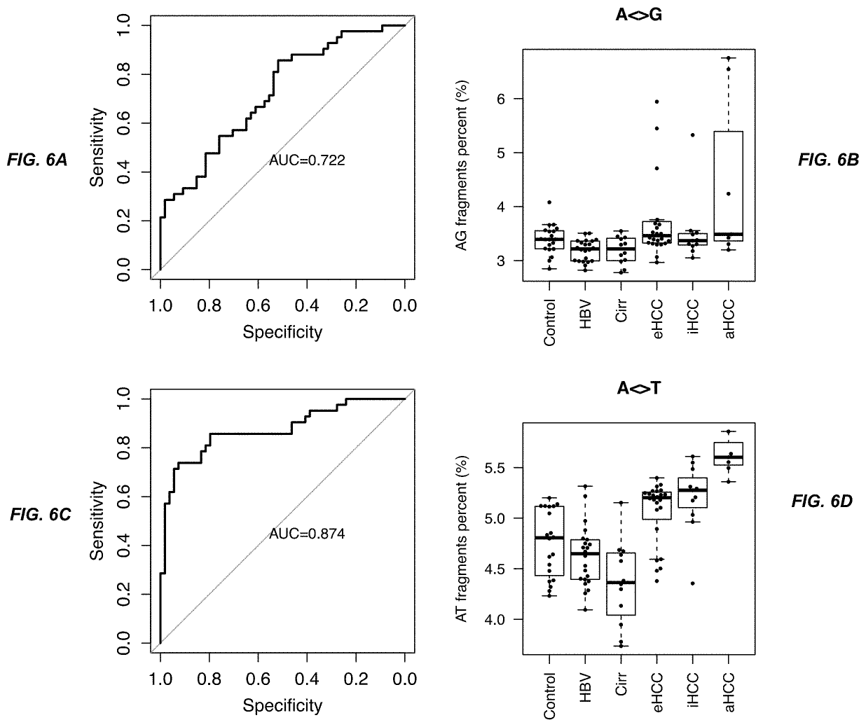

FIGS. 6A-6B shows classification results for the 96 subjects using A< >G fragments according to embodiments of the present disclosure. FIG. 6A shows an ROC curve for the A< >G fragments. FIG. 6B shows a box plot of the percent of A< >G fragments for the six types of subjects. As one can see in FIG. 6B, there is a difference between the 3 non-cancer cohorts and the 3 cancer cohorts, with the cancer subjects generally having a higher A< >G percent. Further, the advanced HCC notably has a statistically significant difference (higher) than the early and intermediate cancer subjects.

FIGS. 6C-6D show classification results for the 96 subjects using A< >T fragments according to embodiments of the present disclosure. FIG. 6C shows an ROC curve for the A< >T fragments. FIG. 6D shows a box plot of the percent of A< >T fragments for the six types of subjects. As one can see in FIG. 6D, there is a pronounced difference between the 3 non-cancer cohorts and the 3 cancer cohorts, with the cancer subjects generally having a higher A< >T percent. Further, the intermediate HCC subjects generally have a higher A< >T percent than the early HCC subjects, and the advanced HCC subjects generally have a higher A< >T percent than the iHCC subjects.

2. Results for C

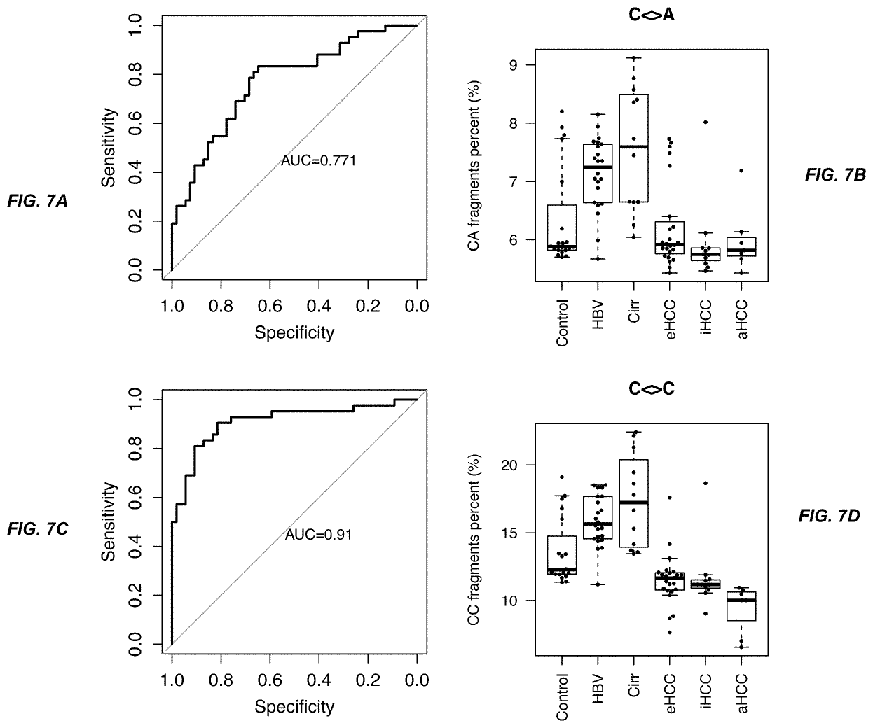

FIGS. 7A-7B show classification results for the 96 subjects using C< >A fragments according to embodiments of the present disclosure. FIG. 7A shows an ROC curve for the C< >A fragments. FIG. 7B shows a box plot of the percent of C< >A fragments for the six types of subjects. As one can see in FIG. 7B, there is a difference between the 3 non-cancer cohorts and the 3 cancer cohorts, with the cancer subjects generally having a lower C< >A percent.

Notably, the HBV subjects and the cirrhosis subjects have a higher C< >A percent than the controls subjects and the cancer subjects. FIG. 7B shows that the biterminal analysis can be used more generally to determine a level of pathology, beyond just cancer. Similarly, A< >C could also be used for such a classification, e.g., as shown in A< >C. Further results for detecting HBV and cirrhosis are provided later.

FIGS. 7C-7D show classification results for the 96 subjects using C< >C fragments according to embodiments of the present disclosure. FIG. 7C shows an ROC curve for the C< >C fragments. FIG. 7D shows a box plot of the percent of C< >C fragments for the six types of subjects. As one can see in FIG. 7D, there is a significant difference between the 3 non-cancer cohorts and the 3 cancer cohorts, with the cancer subjects generally having a lower C< >C percent. The ROC curve in FIG. 7C shows that an embodiment can achieve a specificity of −0.9 while still achieving a sensitivity of −0.8. For the 1-mers, C< >C provides the highest AUC.

In some embodiments, different fragments types can be used together, e.g., to screen for different pathologies or different levels within positive pathologies. For instance, C< >C can be used to screen for cancer, and C< >A can be used to screen for HBV/cirrhosis. If cancer is detected, a different fragment type (e.g., A< >T) can be used to determine the stage of cancer.

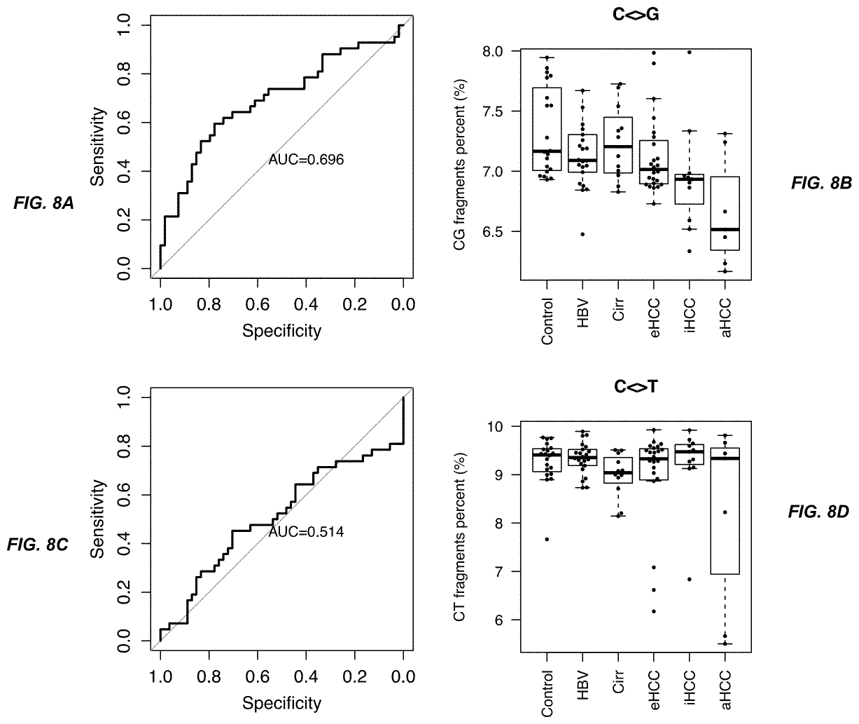

FIGS. 8A-8B show classification results for the 96 subjects using C< >G fragments according to embodiments of the present disclosure. FIG. 8A shows an ROC curve for the C< >G fragments. FIG. 8B shows a box plot of the percent of C< >G fragments for the six types of subjects. As one can see in FIG. 8B, there is some difference between the non-cancer and cancer subjects. The discrimination is somewhat poor for eHCC subjects, but the discrimination between eHCC, iHCC, and aHCC is good. Thus, after a cancer detection (e.g., using C< >C), C< >G could be used to determine the stage of cancer.

FIGS. 8C-8D show classification results for the 96 subjects using C< >T fragments according to embodiments of the present disclosure. FIG. 8C shows an ROC curve for the C< >T fragments. FIG. 8D shows a box plot of the percent of C< >T fragments for the six types of subjects. The results for C< >T are poor.

It is notable that C< >C provides a large AUC for discriminating between cancer and non-cancer, but C< >T performs poorly, while A< >A performs poorly, and A< >T performs quite well.

3. Results for G

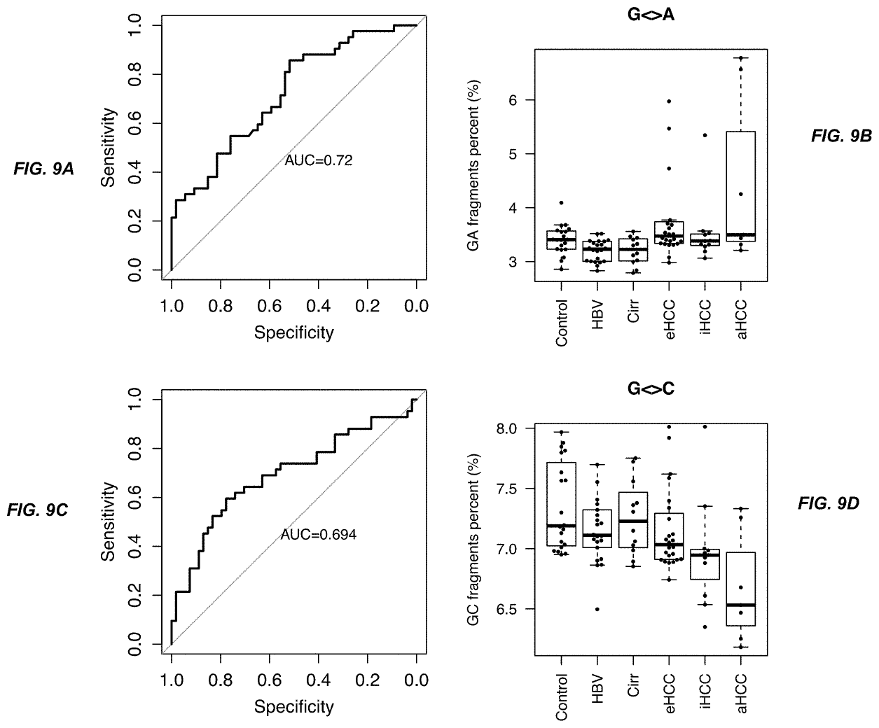

FIGS. 9A-9B show classification results for the 96 subjects using G< >A fragments according to embodiments of the present disclosure. FIG. 9A shows an ROC curve for the G< >A fragments. FIG. 9B shows a box plot of the percent of G< >A fragments for the six types of subjects. The separation between the different cohorts is not as good as other fragment types.

FIGS. 9C-9D show classification results for the 96 subjects using G< >C fragments according to embodiments of the present disclosure. FIG. 9C shows an ROC curve for the G< >C fragments. FIG. 9D shows a box plot of the percent of G< >C fragments for the six types of subjects. As one can see in FIG. 9D, there is some difference between the non-cancer and cancer subjects. The discrimination is somewhat poor for eHCC subjects, but the discrimination between eHCC, iHCC, and aHCC is good. Thus, after a cancer detection (e.g., using C< >C), G< >C could be used to determine the stage of cancer. The performance of G< >C in FIG. 9D is similar to the performance of C< >G in FIG. 8B.

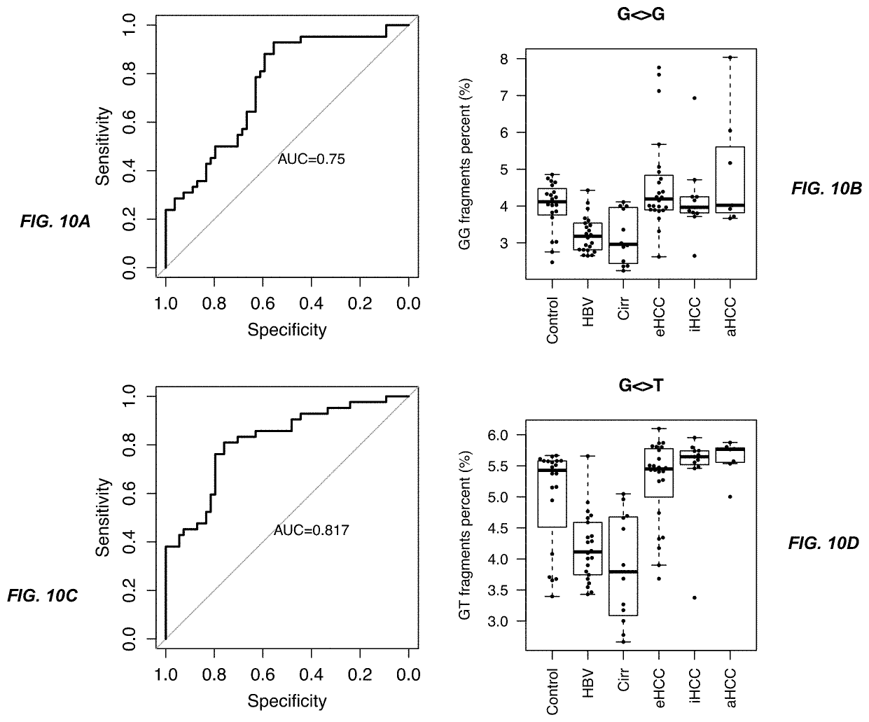

FIGS. 10A-10B show classification results for the 96 subjects using G< >G fragments according to embodiments of the present disclosure. FIG. 10A shows an ROC curve for the G< >G fragments. FIG. 10B shows a box plot of the percent of G< >G fragments for the six types of subjects. A significant increase in sensitivity occurs around 0.6 specificity.

FIGS. 10C-10D show classification results for the 96 subjects using G< >T fragments according to embodiments of the present disclosure. FIG. 10C shows an ROC curve for the G< >T fragments. FIG. 10D shows a box plot of the percent of G< >T fragments for the six types of subjects. The G< >T percent provides decent discrimination between cancer and non-cancer.

4. Results for T

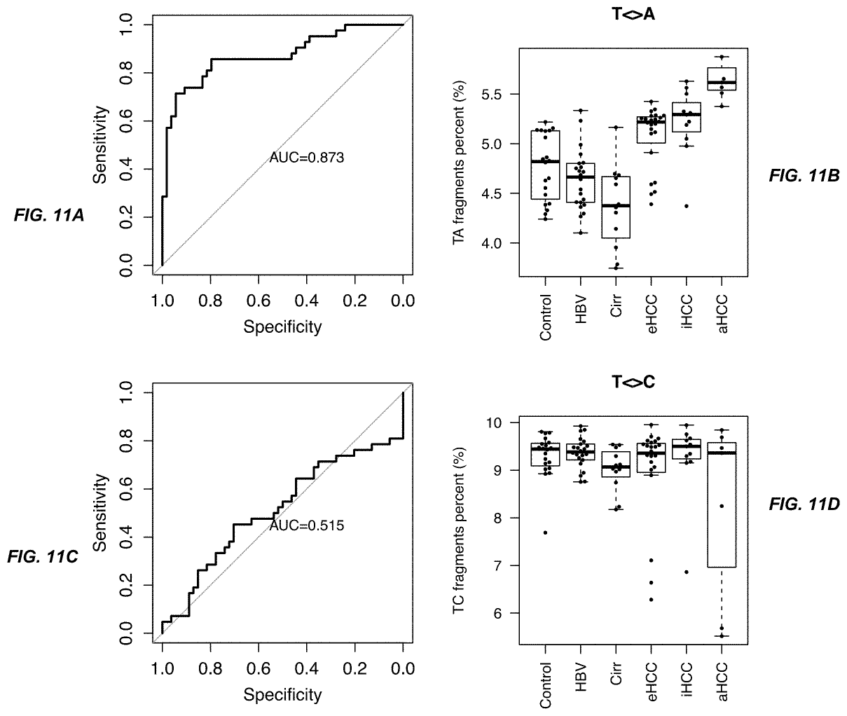

FIGS. 11A-11B show classification results for the 96 subjects using T< >A fragments according to embodiments of the present disclosure. FIG. 11A shows an ROC curve for the T< >A fragments. FIG. 11B shows a box plot of the percent of T< >A fragments for the six types of subjects. The T< >A percent provides good discrimination between cancer and non-cancer, with results comparable to A< >T percent, as shown in FIG. 6D. The discrimination is particularly good between cancer and HBV and cirrhosis. Thus, the parameter of T< >A percent could be used to detect whether a subject has HBV/cirrhosis or cancer. Results for such measurements are provided below.

FIGS. 11C-11D show classification results for the 96 subjects using T< >C fragments according to embodiments of the present disclosure. FIG. 11C shows an ROC curve for the T< >C fragments. FIG. 11D shows a box plot of the percent of T< >C fragments for the six types of subjects. The results for T< >C are poor, similar to the results for C< >T, as in FIG. 8D.

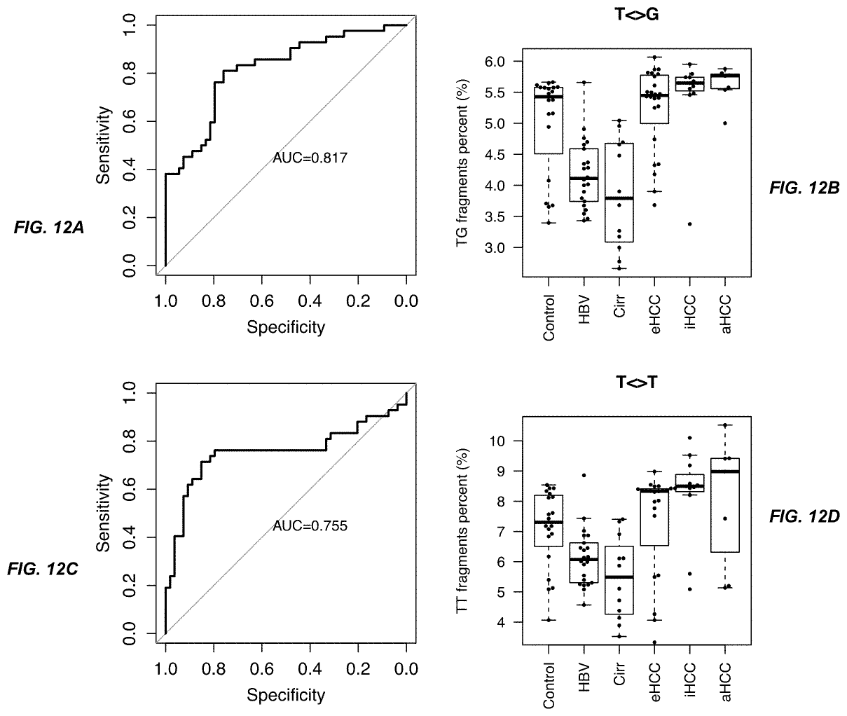

FIGS. 12A-12B show classification results for the 96 subjects using T< >G fragments according to embodiments of the present disclosure. FIG. 12A shows an ROC curve for the T< >G fragments. FIG. 12B shows a box plot of the percent of T< >G fragments for the six types of subjects. The T< >G percent provides decent discrimination between cancer and non-cancer.

FIGS. 12C-12D show classification results for the 96 subjects using T< >T fragments according to embodiments of the present disclosure. FIG. 12C shows an ROC curve for the T< >T fragments. FIG. 12D shows a box plot of the percent of T< >T fragments for the six types of subjects. The T< >T percent provides decent discrimination between cancer and non-cancer until about 0.8 sensitivity, but improvement in sensitivity stall with a drop in specificity.

B. 2-Mer End Motif Pairs in HCC

A similar biterminal analysis can also be done with 2-mers on each end. As described above, such a biterminal analysis would generate 256 different combinations. All 256 combinations of 2-mer end motif pairs were analyzed to determine combinations that provide an AUC>0.9 for the 96 subjects used in the HCC analysis. There are 11 fragment types (2-mer end motif pairs) that provide AUC>0.9.

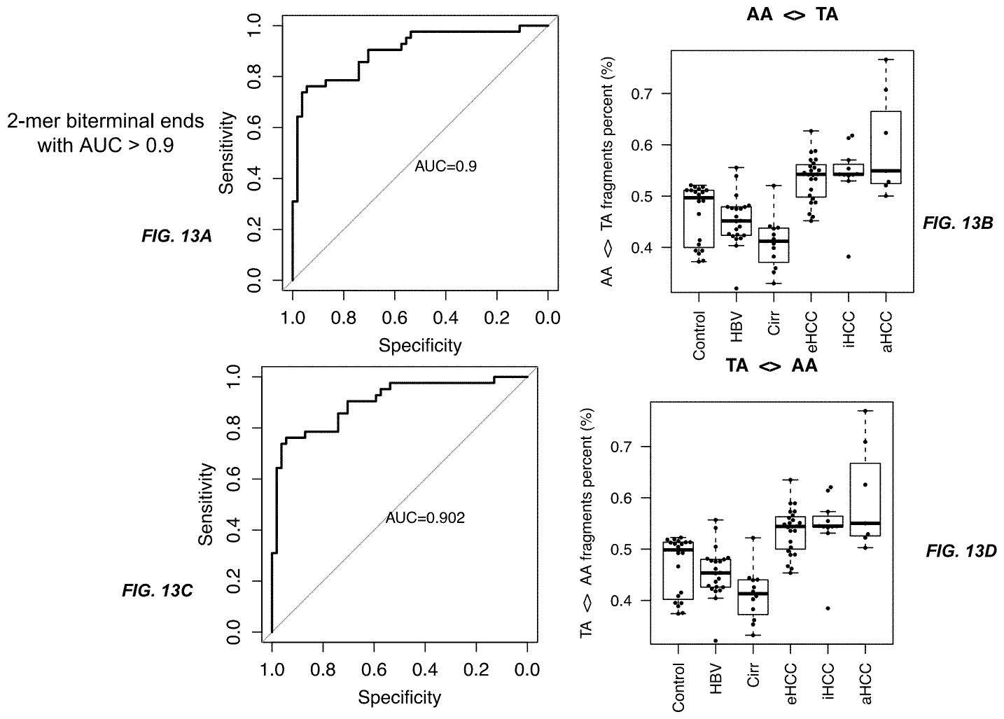

FIGS. 13A-18B show classification results for 2-mer biterminal fragments types that have an AUC>0.9 in distinguishing between non-cancer and HCC according to embodiments of the present disclosure. In these fragment types, AG< >TA fragments have the highest AUC at 0.938. An example fragment type with both high frequency and high AUC is CC< >CC fragment, with a median frequency in the control around 3% and an AUC=0.916.

There are more 2-mer biterminal fragments types that have an AUC>0.9 than 1-mer biterminal fragments types. But given the more combinations, each fragment type occurs with less frequency. The fewer fragments of a given type can impact the amount of sequencing and size of the sample required to achieve a desired statistical accuracy.

1. Results for TA

FIGS. 13A-13B show classification results for the 96 subjects using AA< >TA fragments according to embodiments of the present disclosure. FIG. 13A shows an ROC curve for the AA< >TA fragments. FIG. 13B shows a box plot of the percent of AA< >TA fragments for the six types of subjects. FIGS. 13C-13D show classification results for the 96 subjects using TA< >AA fragments according to embodiments of the present disclosure. FIG. 13C shows an ROC curve for the TA< >AA fragments. FIG. 13D shows a box plot of the percent of TA< >AA fragments for the six types of subjects. The results for AA< >TA and TA< >AA are similar. There is good separation between the cancer and non-cancer subjects, but not as good of separation between the different cancer stages.

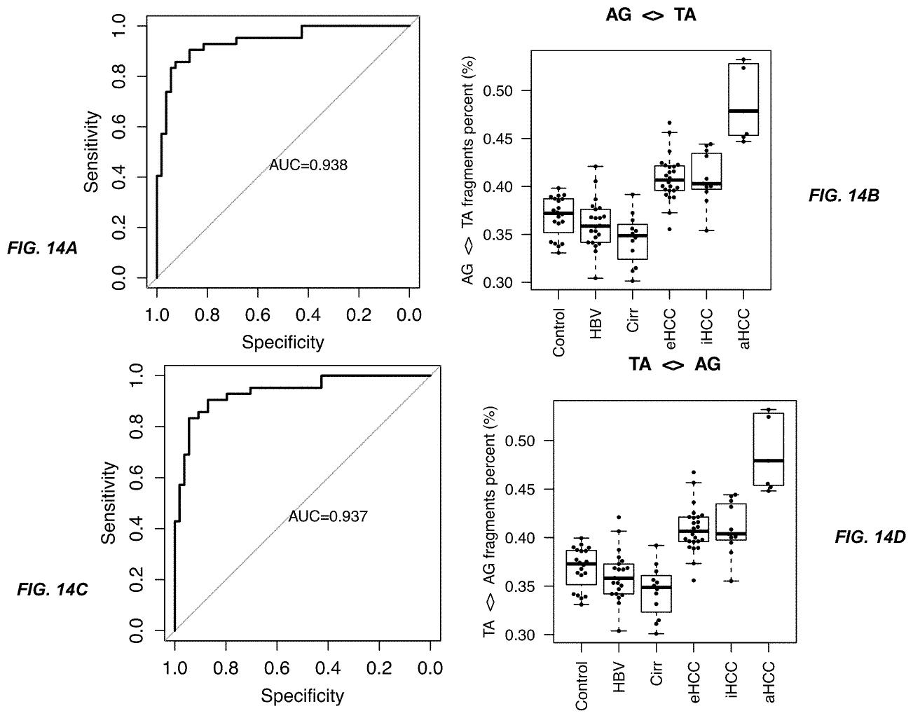

FIGS. 14A-14B show classification results for the 96 subjects using AG< >TA fragments according to embodiments of the present disclosure. FIG. 14A shows an ROC curve for the AG< >TA fragments. FIG. 14B shows a box plot of the percent of AG< >TA fragments for the six types of subjects. FIGS. 14C-14D show classification results for the 96 subjects using TA< >AG fragments according to embodiments of the present disclosure. FIG. 14C shows an ROC curve for the TA< >AG fragments. FIG. 14D shows a box plot of the percent of TA< >AG fragments for the six types of subjects.

The results for AG< >TA and TA< >AG are similar. There is good separation between the cancer and non-cancer subjects. There is also good separation between aHCC and the other two cancer classifications (eHCC and iHCC). Thus, these fragment types can be used to accurately identify aHCC subjects, as well as screen for cancer.

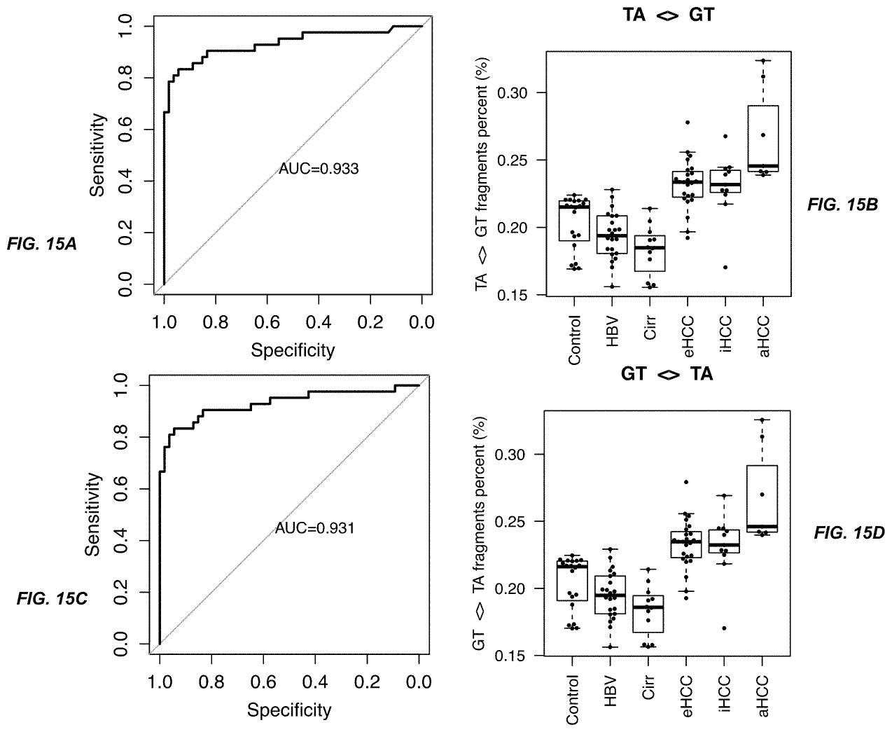

FIGS. 15A-15B show classification results for the 96 subjects using TA< >GT fragments according to embodiments of the present disclosure. FIG. 15A shows an ROC curve for the TA< >GT fragments. FIG. 15B shows a box plot of the percent of TA< >GT fragments for the six types of subjects. FIGS. 15C-15D show classification results for the 96 subjects using GT< >TA fragments according to embodiments of the present disclosure. FIG. 15C shows an ROC curve for the GT< >TA fragments. FIG. 15D shows a box plot of the percent of GT< >TA fragments for the six types of subjects.

The results for TA< >GT and GT< >TA are similar. There is good separation between the cancer and non-cancer subjects. There is also good separation between aHCC and the other two cancer classifications (eHCC and iHCC), as although not as good as for AG< >TA and TA< >AG. Thus, these fragment types can be used to identify aHCC subjects, as well as screen for cancer.

2. Results for CC

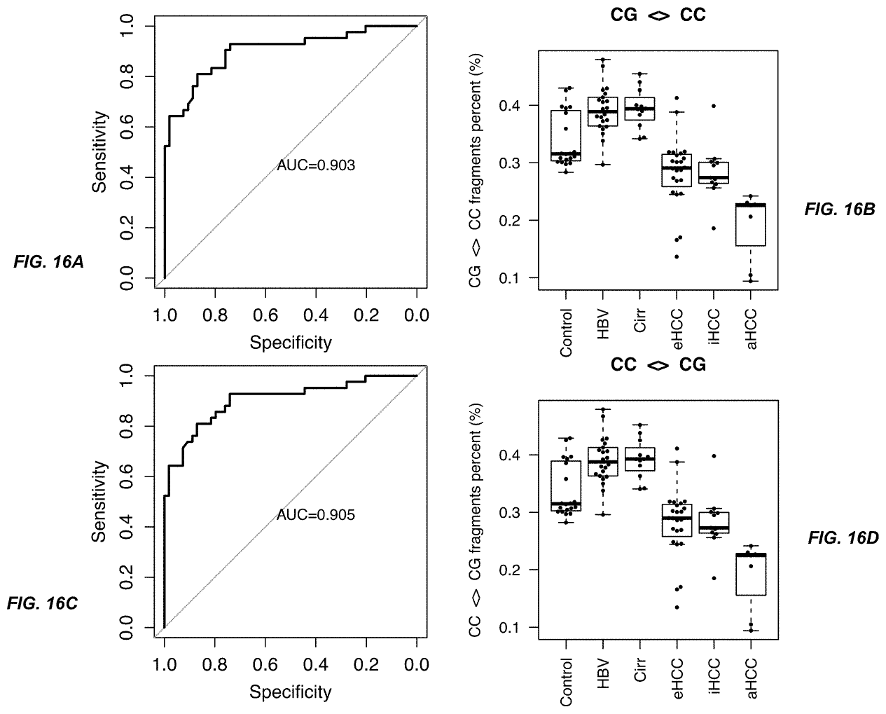

FIGS. 16A-16B show classification results for the 96 subjects using CG< >CC fragments according to embodiments of the present disclosure. FIG. 16A shows an ROC curve for the CG< >CC fragments. FIG. 16B shows a box plot of the percent of CG< >CC fragments for the six types of subjects. FIGS. 16C-16D show classification results for the 96 subjects using CC< >CG fragments according to embodiments of the present disclosure. FIG. 16C shows an ROC curve for the CC< >CG fragments. FIG. 16D shows a box plot of the percent of CC< >CG fragments for the six types of subjects.

The results for CG< >CC and CC< >GC are similar. There is good separation between the cancer and non-cancer subjects. There is also good separation between aHCC and the other two cancer classifications (eHCC and iHCC). Thus, these fragment types can be used to identify aHCC subjects, as well as screen for cancer.

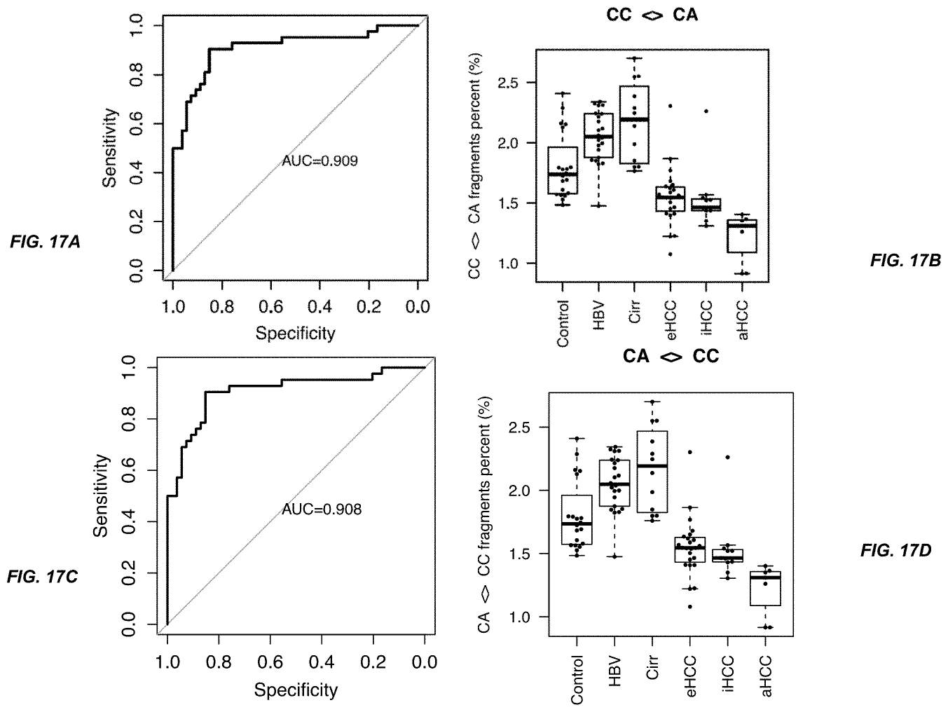

FIGS. 17A-17B show classification results for the 96 subjects using CC< >CA fragments according to embodiments of the present disclosure. FIG. 17A shows an ROC curve for the CC< >CA fragments. FIG. 17B shows a box plot of the percent of CC< >CA fragments for the six types of subjects. FIGS. 17C-17D show classification results for the 96 subjects using CA< >CC fragments according to embodiments of the present disclosure. FIG. 17C shows an ROC curve for the CA< >CC fragments. FIG. 17D shows a box plot of the percent of CA< >CC fragments for the six types of subjects.

The results for CC< >CA and CA< >CC are similar. There is good separation between the cancer and non-cancer subjects. There is also decent separation between aHCC and the other two cancer classifications (eHCC and iHCC). Thus, these fragment types can be used to identify aHCC subjects, as well as screen for cancer.

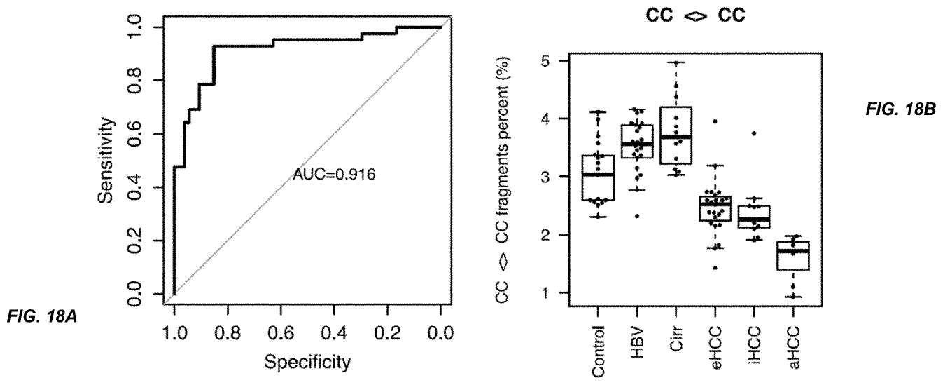

FIGS. 18A-18B show classification results for the 96 subjects using CC< >CC fragments according to embodiments of the present disclosure. FIG. 18A shows an ROC curve for the CC< >CC fragments. FIG. 18B shows a box plot of the percent of CC< >CC fragments for the six types of subjects. There is good separation between the cancer and non-cancer subjects. There is also decent separation between aHCC and the other two cancer classifications (eHCC and iHCC). Thus, these fragment types can be used to identify aHCC subjects, as well as screen for cancer.

An advantage of CC< >CC is that that these fragments generally comprise between 1-5% of all cfDNA in a plasma sample, thereby providing a large number of DNA fragments from a relatively small sample. For example, 500,000 DNA fragments can provide sufficient accuracy, thereby allowing a small sample amount (e.g., less than 1 ng DNA or 1 microliter of DNA solution extracted from plasma) to be used. For instance, 500 hundred thousand fragments of 200 bp (typical in plasma) equals about 0.3× of the human genome. 1 mL of plasma as about 1,000 to 5,000 genome-equivalents of DNA. On average, each genome is fragmented into millions of pieces of DNA. Even for larger samples, less sequencing can be performed. But even for other fragment types that have a smaller frequency, such fragments are still plentiful in a standard sequencing run since the fragments of a particular type can be from anywhere in a genome. The relationship of the number of fragments and accuracy is explored in a later section.

C. 2-Mer End Motif Pairs Using Bases on Either Side of Cutting Site

As described above, bases on either side of the cutting site can be used. The bases on the other side of the cutting site can be labeled using lowercase, and the bases on the fragment can be labeled using uppercase. The use of off-fragment bases can reflect instances where the fragmentation is dependent on the bases on both sides of the cutting site.

The nucleotide information at the −1, −2, −3 etc. positions can be informative and enhance the performance of biterminal analysis. The nucleotide information can be obtained after alignment of the sequenced fragment back to the reference genome. In one embodiment, the nucleotide at the −1 and +1 position on each end was used to categorized fragment types. Nucleotides in the negative positions are denoted in lower case here for clarity. A vertical line (|) denotes the cutting site at the ends of fragments). Although the −1 and +1 positions are used, the positions do not have to be consecutive, e.g., −2 and +1 could be used.

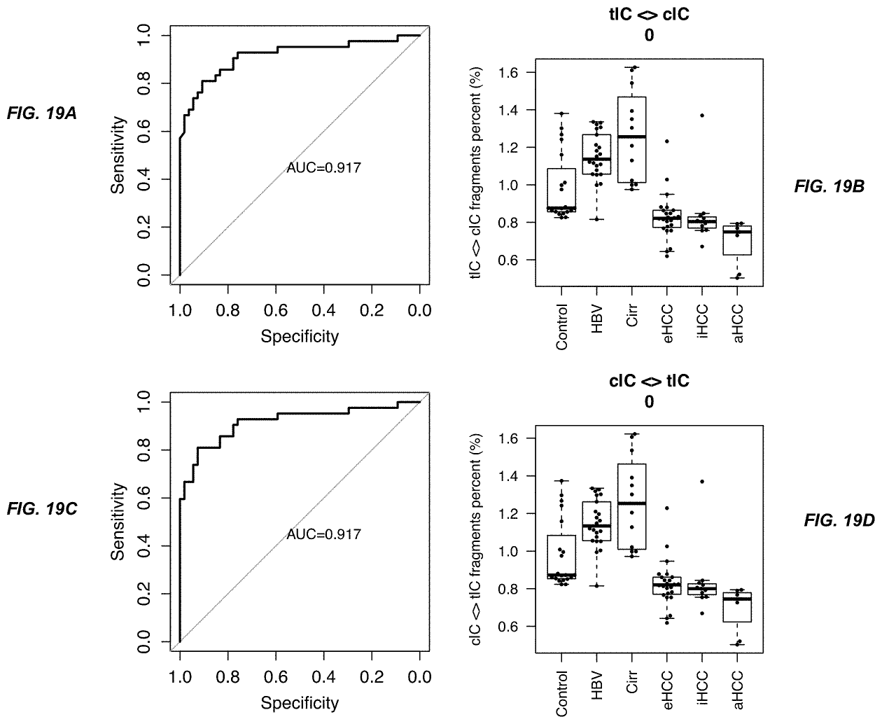

FIGS. 19A-19B show the performance of a biterminal analysis with a −1 and +1 position nucleotides in distinguishing HCC according to embodiments of the present disclosure. FIGS. 19A-19B show classification results using t|C< >c|C fragments according to embodiments of the present disclosure. FIG. 19A shows an ROC curve for the t|C< >c|C fragments. FIG. 19B shows a box plot of the percent of t|C< >c|C fragments for the six types of subjects. FIGS. 19C-19D show classification results using c|C< >t|C fragments according to embodiments of the present disclosure. FIG. 19C shows an ROC curve for the c|C< >t|C fragments. FIG. 19D shows a box plot of the percent of c|C< >t|C fragments for the six types of subjects.

The results for t|C< >c|C and c|C< >t|C are similar and are the best performing −1, +1 types. Including the −1 and +1 positions in biterminal analysis of the HCC dataset achieves discrimination between HCC and non-cancer with an AUC=0.917 in t|C< >c|C and c|C< >t|C fragments. The frequency of such fragments is also somewhat higher than most of the 2-mer fragment types when the bases are on the fragment.

D. HBV and Cirrhosis

Some embodiments can detect levels of other pathologies besides cancer, as mentioned above. For the liver, such pathologies include chronic hepatitis caused by HBV and cirrhosis. Motifs with the highest AUC in distinguishing control vs chronic hepatitis due to HBV and control vs cirrhosis are provide in Table 1 below. Some example ROC curves follow.

TABLE 1
End motif pairs with the highest AUC in distinguishing control vs
HBV, control vs cirrhosis

| | Distinguishing Control | | Distinguishing Control vs | |
|---|---|---|---|---|
| | vs HBV | | Cirrhosis | |
| | Motif with highest | Highest | Motif(s) with | Highest |
| Motif type | AUC | AUC | highest AUC | AUC |
| 2end: −1+1 | a\|G <> a\|G | 0.814 | t\|C <> t\|C | 0.867 |
| | | | g\|T <> a\|G | |
| | | | a\|G <> g\|T | |
| | | | g\|G <> a\|T | |
| | | | a\|T <> g\|G | |
| 2end: +2 | CG <> AA | 0.864 | GC <> TA | 0.871 |
| | | | TA <> GC | |
| 2end: +1 | G <> G | 0.807 | C <> C | 0.867 |
| | | | C <> A | 0.862 |
| | | | A <> C | 0.858 |
| | | | G <> T | 0.858 |
| | | | T <> G | 0.858 |

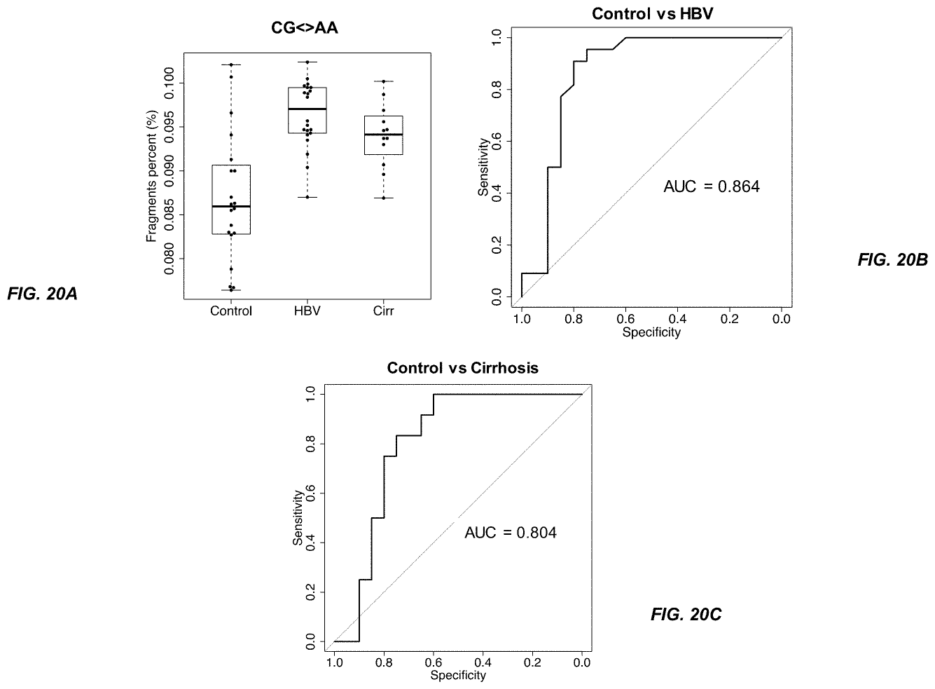

FIGS. 20A-20C provide the performance of CG< >AA in distinguishing controls from HBV and cirrhosis according to embodiments of the present disclosure. FIG. 20A is a box plot for CG< >AA, showing separation between controls and HBV, as well as cirrhosis. FIG. 20B shows an ROC curve for CG< >AA distinguishing control and HBV, with an AUC of 0.864, which was the best 2end:+2 end motif pair for HBV. FIG. 20C shows an ROC curve for CG< >AA distinguishing control and cirrhosis, with an AUC of 0.804.

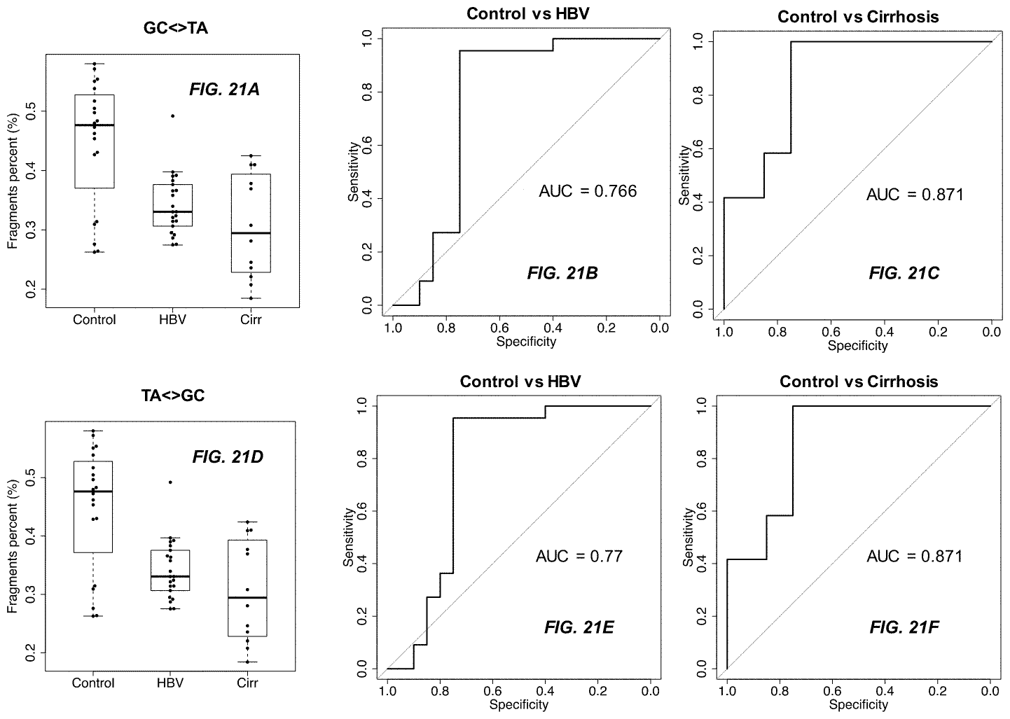

FIGS. 21A-21C provide the performance of GC< >TA in distinguishing controls from HBV and cirrhosis according to embodiments of the present disclosure. FIG. 21A is a box plot for GC< >TA, showing separation between controls and cirrhosis, as well as HBV. FIG. 21B shows an ROC curve for GC< >TA distinguishing control and HBV, with an AUC of 0.766. FIG. 21C shows an ROC curve for GC< >TA distinguishing control and cirrhosis, with an AUC of 0.871, which was tied for the best 2end:+2 end motif pair for cirrhosis.

FIGS. 21D-21F provide the performance of TA< >GC in distinguishing controls from HBV and cirrhosis according to embodiments of the present disclosure. FIG. 21D is a box plot for TA< >GC, showing separation between controls and cirrhosis, as well as HBV. FIG. 21E shows an ROC curve for TA< >GC distinguishing control and HBV, with an AUC of 0.77. FIG. 21F shows an ROC curve for TA< >GC distinguishing control and cirrhosis, with an AUC of 0.871, which was tied for the best 2end:+2 end motif pair for cirrhosis.

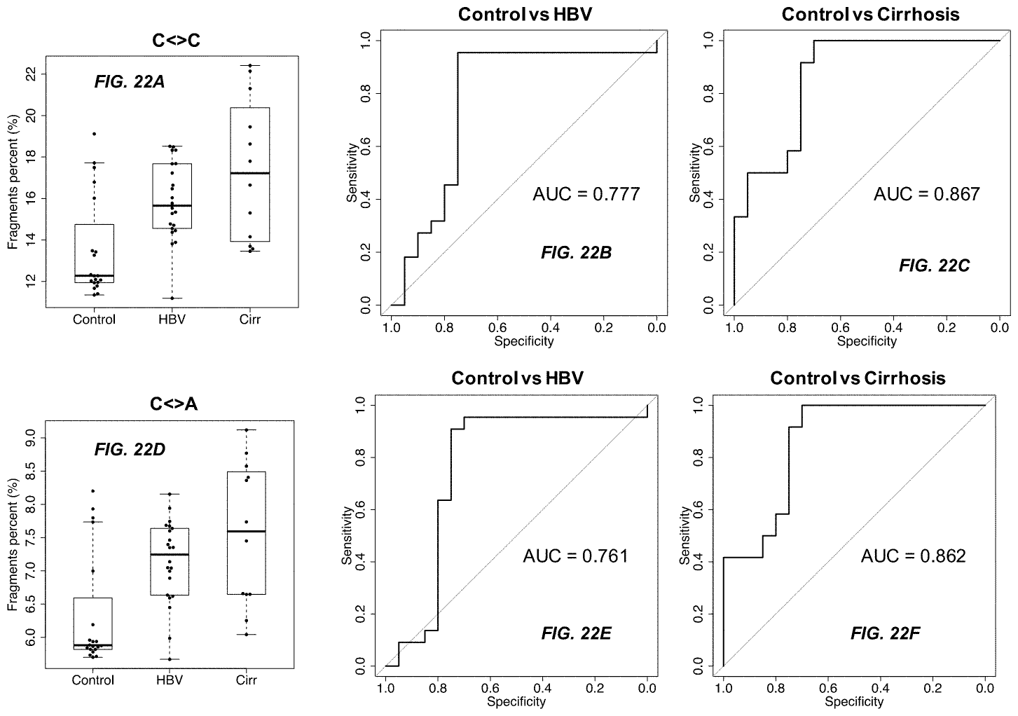

FIGS. 22A-22C provide the performance of C< >C in distinguishing controls from HBV and cirrhosis according to embodiments of the present disclosure. FIG. 22A is a box plot for C< >C, showing separation between controls and cirrhosis, as well as HBV. FIG. 22B shows an ROC curve for C< >C distinguishing control and HBV, with an AUC of 0.777. FIG. 22C shows an ROC curve for C< >C distinguishing control and cirrhosis, with an AUC of 0.867.

FIGS. 22D-22F provide the performance of C< >A in distinguishing controls from HBV and cirrhosis according to embodiments of the present disclosure. FIG. 22D is a box plot for C< >A, showing separation between controls and cirrhosis, as well as HBV. FIG. 22F shows an ROC curve for C< >A distinguishing control and HBV, with an AUC of 0.761. FIG. 22F shows an ROC curve for C< >A distinguishing control and cirrhosis, with an AUC of 0.862.

E. Examples of Other End Motif Pairs and Parameters (Aggregate Values)

As shown above for the end motif pairs of different fragment types, different combinations with different N-mers may result in better performance. Some other examples could be tt|CC< >ct|CC or a|CCC< >ct|CG.

Further, the proportions of different fragment types may be combined, e.g., by summing the individual values, determining a statistical value (e.g., a mean, average, weighted average, a median, or mode), or used as inputs to a machine learning model. For instance, each of a set of fragment types can form one dimension of a vector that represents a multidimensional data point. The data points for different classifications can form clusters, where a new data point for a new sample can be assigned to a cluster based on a vector distance (e.g., a difference in the fragment type proportions) from the centroid of each cluster. Various other models can be used, such as support vector machines, decision trees, neural networks, etc.

### III. Pathologies of Other Tissues

The end motif pairs can be used to screen for other cancers as well. As examples of other cancers, colorectal cancer (CRC), lung squamous cell carcinoma (LUSC), nasopharyngeal cancer (NPC), and head and neck squamous cell carcinoma (HNSCC) are used. These cancers provide a good representation of common cancers that can be detected.

We sequenced 30 additional control samples and 40 plasma DNA samples of other cancer types (10 colorectal carcinoma (CRC), 10 lung squamous cell carcinoma (LUSC), 10 nasopharyngeal carcinoma (NPC), and 10 head and neck squamous cell carcinoma (HNSCC)) to a median paired-read of 42 million (range: 19-65 million).

A. CC< >CC

Given that CC< >CC performed well and this fragment type was prevalent in plasma samples, we tested the potential of biterminal analysis with CC< >CC % in other types cancers.

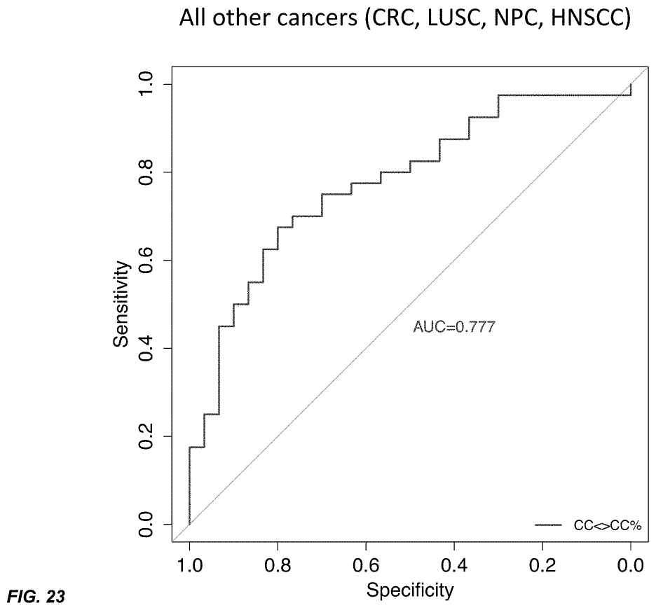

FIGS. 23-25B show ROC curves of CC< >CC fragment proportions and AUC values in distinguishing between controls and other cancers such as colorectal cancer (CRC), lung squamous cell carcinoma (LUSC), nasopharyngeal cancer (NPC), and head and neck squamous cell carcinoma (HNSCC) according to embodiments of the present disclosure. In distinguishing non-cancer from these other four types of cancer combined, the AUC is 0.77, as shown in FIG. 23. The accuracy in the ROC curve, including the AUC, is determined for discriminating whether a subject has cancer or not.

We also analyzed each of these four types of cancers individually. An ROC curve and AUC is provided for discriminating between the control and the particular type of cancer.

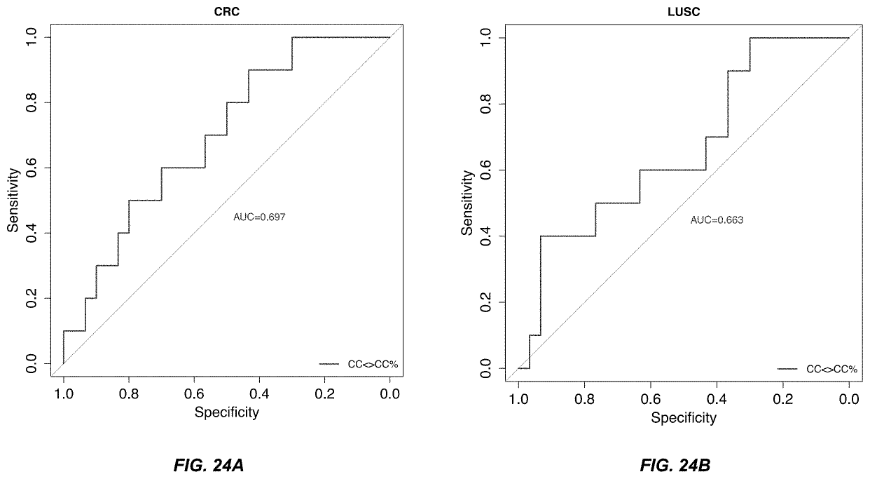

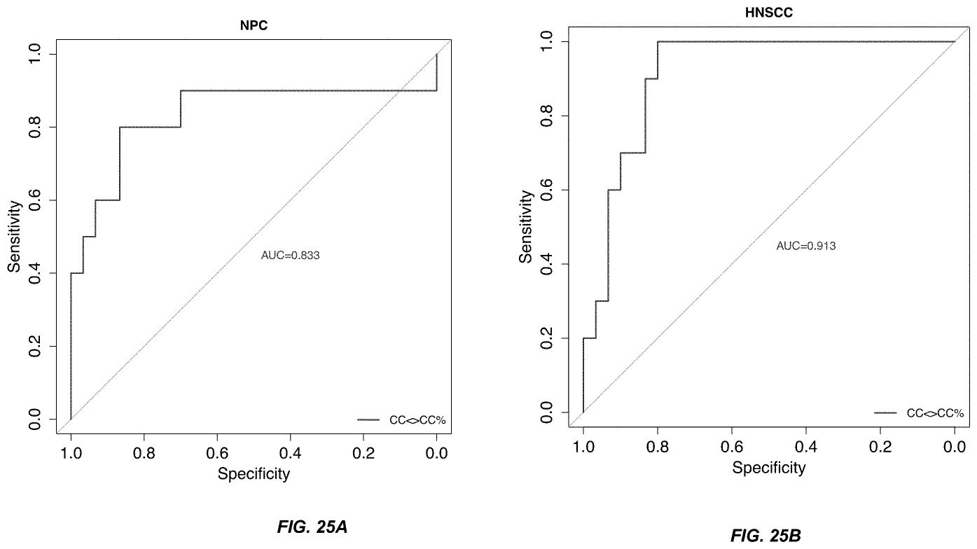

FIG. 24A shows the ROC curve of CC< >CC fragment proportions and AUC values in distinguishing between controls and CRC according to embodiments of the present disclosure. FIG. 24B shows the ROC curve of CC< >CC fragment proportions and AUC values in distinguishing between controls and LUSC according to embodiments of the present disclosure. FIG. 25A shows the ROC curve of CC< >CC fragment proportions and AUC values in distinguishing between controls and NPC according to embodiments of the present disclosure. FIG. 25B shows the ROC curve of CC< >CC fragment proportions and AUC values in distinguishing between controls and HNSCC according to embodiments of the present disclosure. When separated by each individual cancer type, the AUC for differentiating HNSCC is 0.913, NPC is 0.833, CRC is 0.697, and LUSC is 0.663.

B. −1 and +1 position

We also analyzed the use of off-fragment bases, specifically the −1 position, in combination with the +1 position. Examples including −1 position nucleotide in biterminal analysis for distinguishing these four other cancers are provided below.

1. Results for tIC

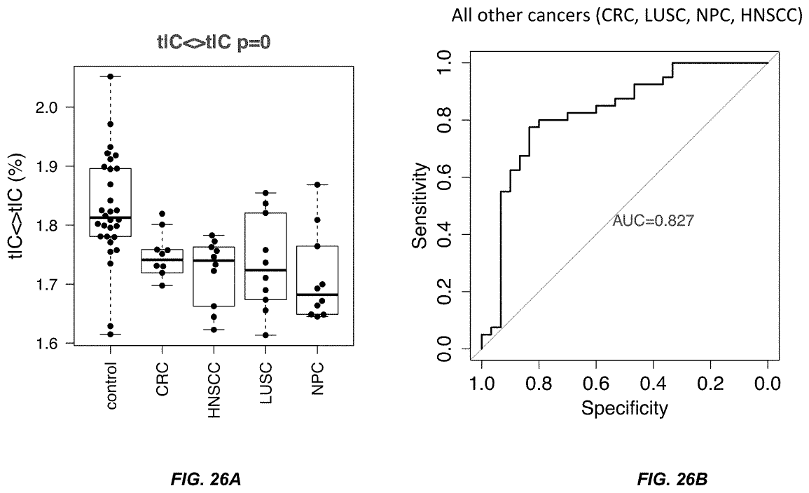

FIGS. 26A-28B show the performance of three example biterminal fragments with −1 and +1 position nucleotides in distinguishing other cancers (CRC, LUSC, NPC, HNSCC) according to embodiments of the present disclosure. Each of the three examples involve t|C at one end or two ends. For t|C< >t|C %, the AUC is 0.827. For t|C< >a|C, the AUC is 0.83. For a|C< >t|C %, the AUC is 0.83. These are the three best performing end motif pairs of this type. Including the −1 position in biterminal analysis enhances the discrimination of other cancer types. In distinguishing non-cancer from these other four cancer types (CRC, LUSC, NPC, HNSCC), the proportions of some fragment types perform better than using CC< >CC %.

FIG. 26A shows a box plot of t|C< >t|C percent for controls, CRC, LUSC, NPC, and HNSCC according to embodiments of the present disclosure. Each of these four cancers have generally lower values for the t|C< >t|C percent. FIG. 26B shows the ROC curve and AUC (0.827) for t|C< >t|C fragments.

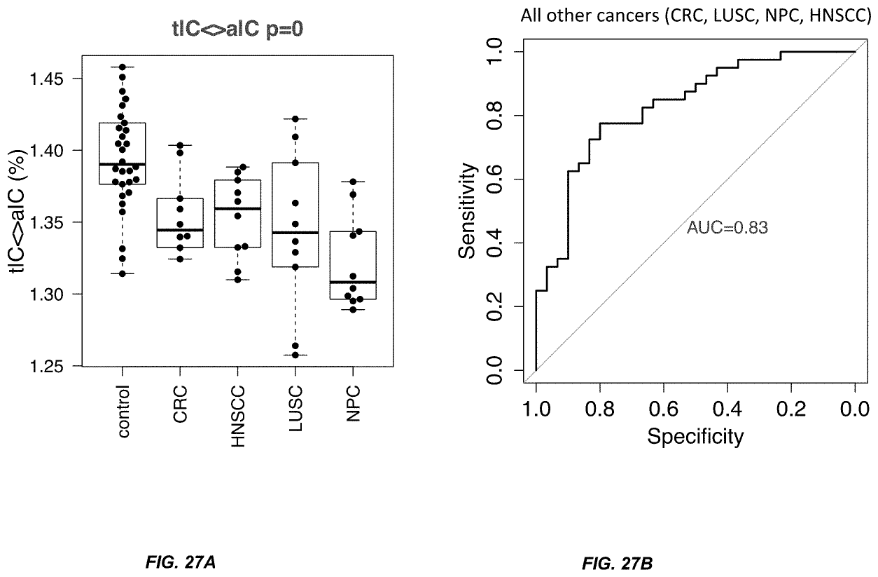

FIG. 27A shows a box plot of t|C< >a|C percent for controls, CRC, LUSC, NPC, and HNSCC according to embodiments of the present disclosure. Each of these four cancers have generally lower values for the t|C< >a|C percent. FIG. 27B shows the ROC curve and AUC (0.83) for t|C< >a|C fragments.

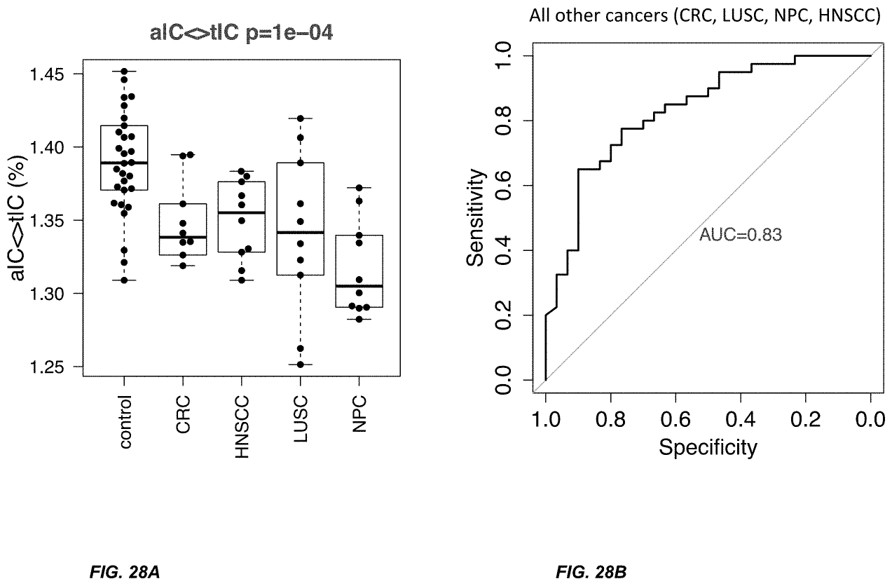

FIG. 28A shows a box plot of a|C< >t|C percent for controls, CRC, LUSC, NPC, and HNSCC according to embodiments of the present disclosure. Each of these four cancers have generally lower values for the a|C< >t|C percent. FIG. 28B shows the ROC curve and AUC (0.83) for a|C< >t|C fragments.

2. Best Results for Each Cancer

When each cancer type is analyzed individually, different fragment types can achieve the highest performance for the different cancers.

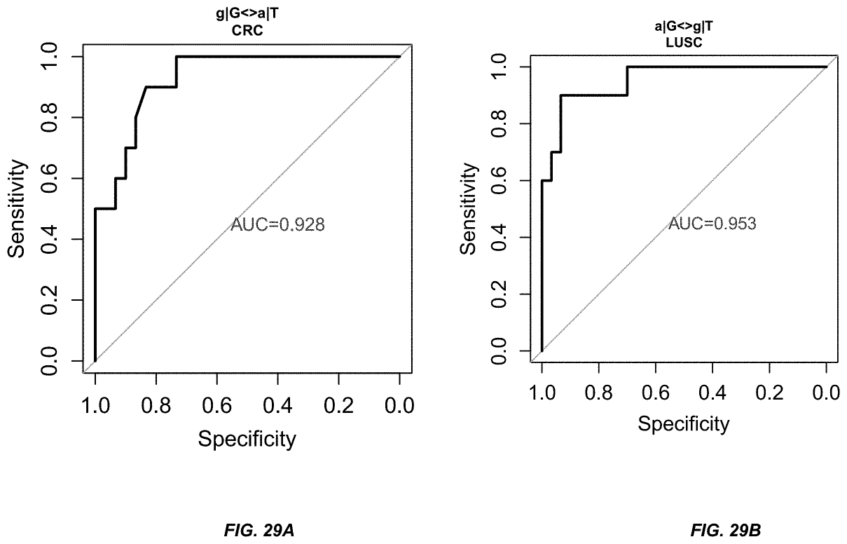

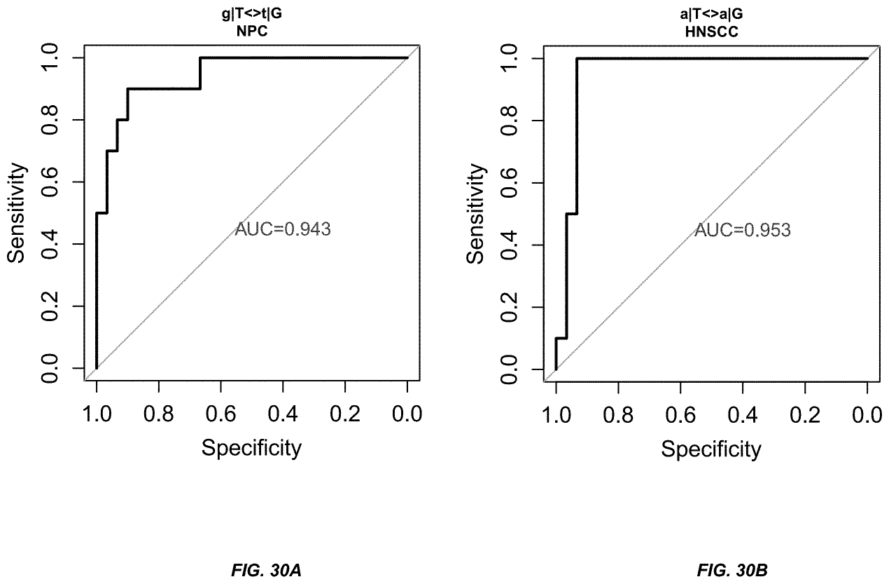

FIGS. 29A-30B show the best performance for respective biterminal fragments with −1 and +1 position nucleotides in distinguishing each of CRC, LUSC, NPC, or HNSCC according to embodiments of the present disclosure. FIG. 29A shows the ROC curve and AUC of g|G< >a|T fragments for CRC according to embodiments of the present disclosure. FIG. 29B shows the ROC curve and AUC of a|G< >g|T fragments for LUSC according to embodiments of the present disclosure. FIG. 30A shows the ROC curve and AUC of g|T< >t|G fragments for NPC according to embodiments of the present disclosure. FIG. 30B shows the ROC curve and AUC of a|T< >a|G fragments for HNSCC according to embodiments of the present disclosure.

The g|G< >a|T fragment percentages distinguishes CRC from non-cancer with an AUC of 0.928 (FIG. 29A); a|G< >g|T fragment percentages distinguishes LUSC from non-cancer with an AUC of 0.953 (FIG. 29B); g|T< >t|G fragment percentages distinguishes NPC from non-cancer with an AUC of 0.943 (FIG. 30A); and a|T< >a|G fragment percentages distinguishes HNSCC from non-cancer with an AUC of 0.953 (FIG. 30B).

### IV. Distinguishing Among Different Stages of Pathology

Some embodiments can distinguish among different stages of pathology (e.g., cancer). Such distinctions can be performed in a second pass using a second set of end motif pair(s), e.g., where a first pass is performed to distinguish between whether the subject has the pathology. For instance, C< >C can be used in a first pass that determine whether cancer exists. Then, A< >T can be used to differentiate between early, intermediate, and advanced stages of cancer. Further, different sets of end motif pair(s) can be used to differentiate between different stages of cancer. Thus, various models (e.g., each with a different end motif pair) can be used collectively or as a single model (e.g., a decision tree) to determine the stage of the pathology.

A. HCC

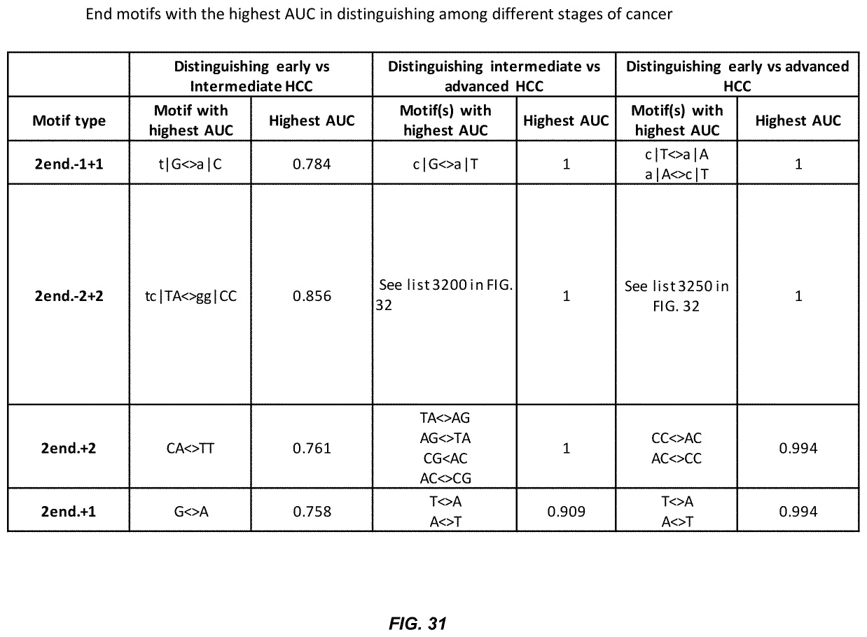

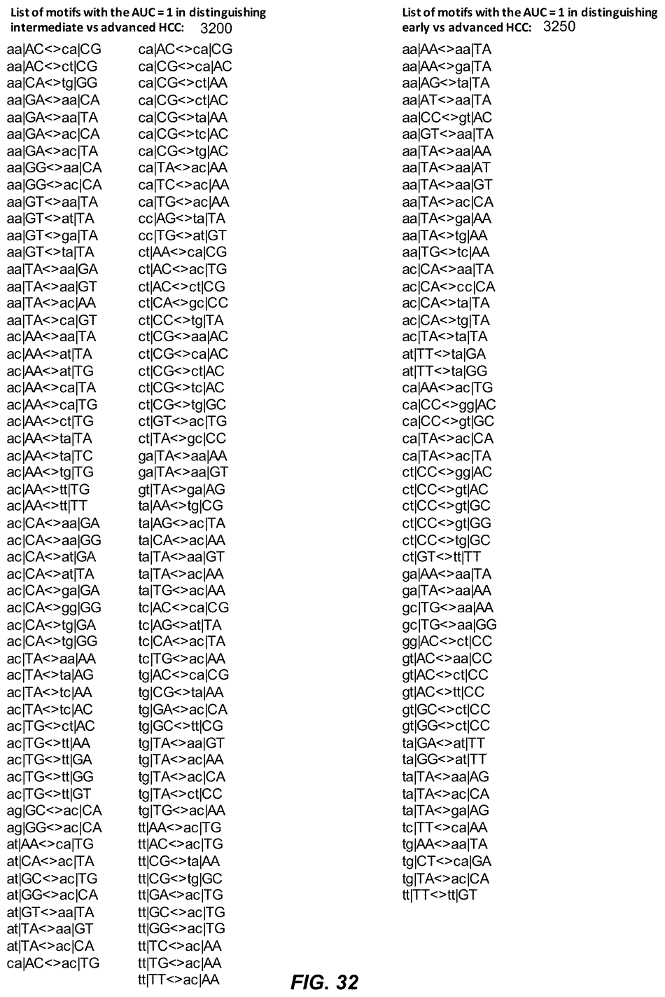

FIG. 31 shows a table including performance results of the end motifs with the highest AUC in distinguishing among different stages of cancer according to embodiments of the present disclosure. The results show the accuracy for distinguishing among the three stages of cancer, namely (a) distinguishing early vs. intermediate HCC, (b) distinguishing intermediate vs. advanced HCC; and (c) distinguishing early vs. advanced HCC. The motif type lists four different classes of fragment types: (1) 2end:−1+1; (2) 2end:−2+2; (3) 2end:+2; and (4) 2end:+1. The best performing end motif pair(s) are provided for each motif type and for each pairwise distinction between cancer stages. Some of the AUC are 1, showing 100% accuracy. The distinctions between early/intermediate and the advanced HCC can be done with 100% accuracy, with many options available for distinguishing intermediate vs. advanced HCC. Some of the end motif pairs are provided in FIG. 32.

FIG. 32 shows a list 3200 of all 2end:−2+2 types with 100% accuracy for distinguishing between intermediate and advanced HCC and a list 3250 of all 2end:−2+2 types with 100% accuracy for distinguishing between early and advanced HCC.

Graphs of the performance of some of the best performing 2end:−1+1 end motif types are provided below.

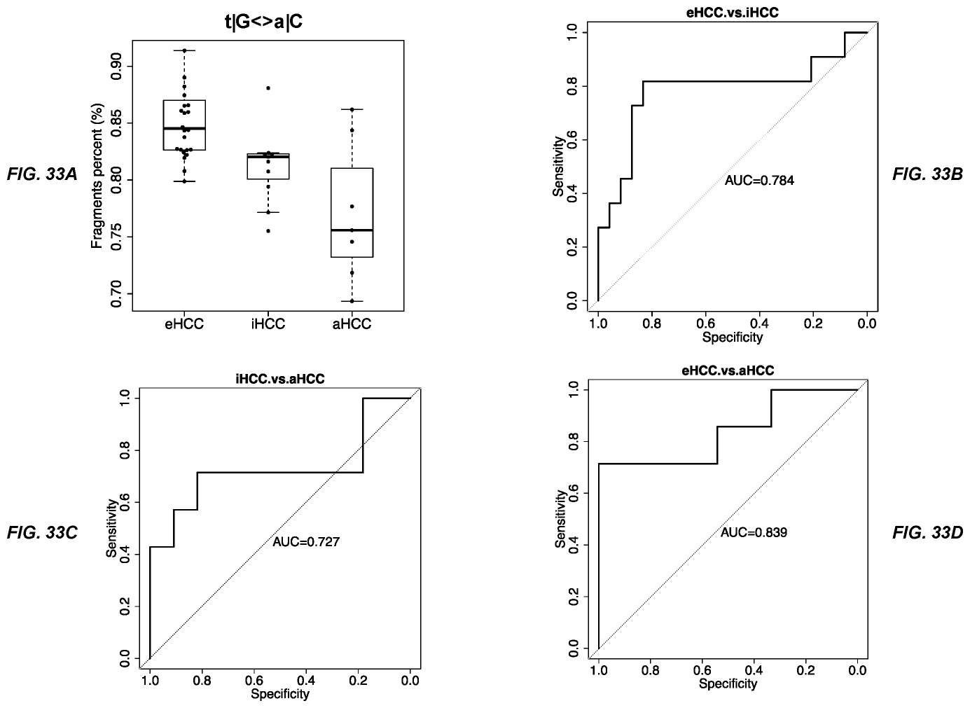

FIGS. 33A-33D provide performance results for the best performing biterminal −1 and +1 position motifs in distinguishing early vs intermediate HCC. FIG. 33A shows a box plot of t|G< >a|C % for the three HCC stages. As shown, the t|G< >a|C % progressively decreases with the stage of cancer. In some embodiments, a calibration function can be determined using the median or mean values for each classification, thereby allowing for more classifications, e.g., as a continuum between the stages. Such a calibration function can be used with any end motif pair(s). FIG. 33B shows an ROC curve using t|CT< >a|C to distinguish between eHCC and iHCC. FIG. 33C shows an ROC curve using t|CT< >a|C to distinguish between iHCC and aHCC. FIG. 33D shows an ROC curve using t|CT< >a|C to distinguish between eHCC and aHCC.

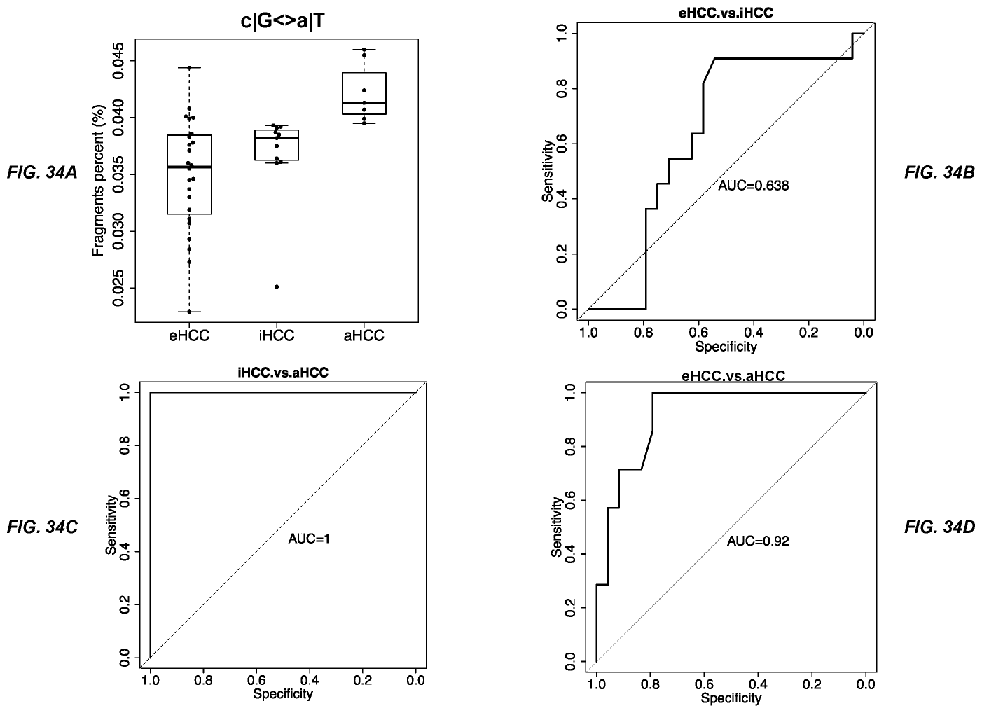

FIGS. 34A-34D provide performance results for the best performing biterminal −1 and +1 position motifs in distinguishing intermediate vs advanced HCC. FIG. 34A shows a box plot of c|G< >a|T % for the three HCC stages. As shown, the c|G< >a|T % progressively increases with the stage of cancer. FIG. 34B shows an ROC curve using c|CT< >a|T to distinguish between eHCC and iHCC. FIG. 34C shows an ROC curve using c|CT< >a|T to distinguish between iHCC and aHCC, with an AUC of 1 achieved. FIG. 34D shows an ROC curve using c|CT< >a|T to distinguish between eHCC and aHCC.

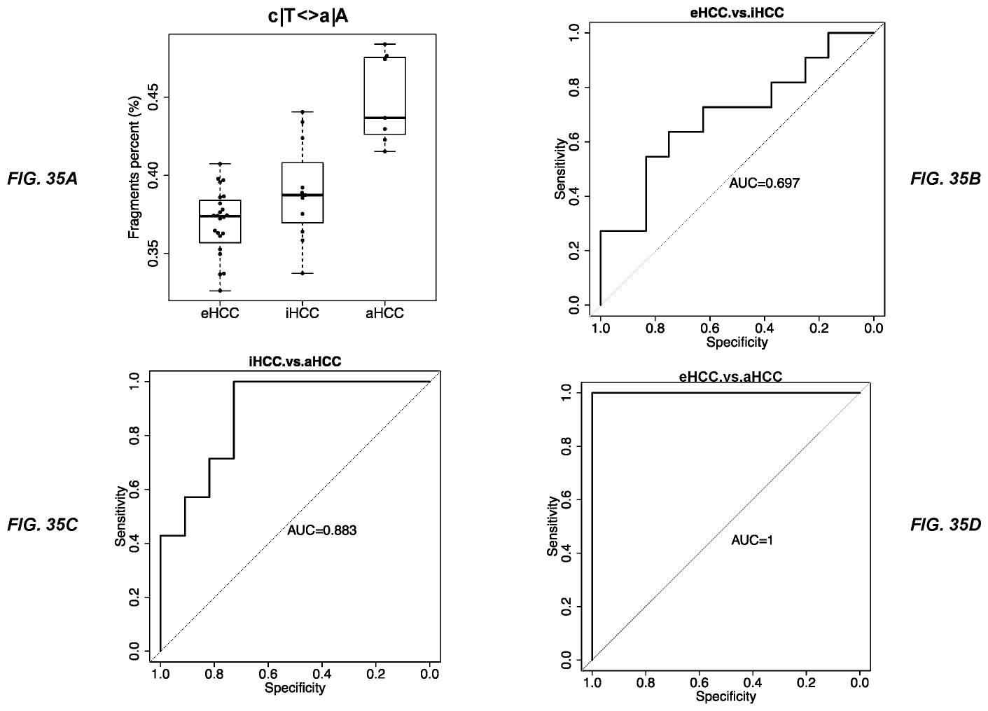

FIGS. 35A-35D provide performance results for the best performing biterminal −1 and +1 position motifs in distinguishing early vs advanced HCC. FIG. 35A shows a box plot of c|T< >a|A % for the three HCC stages. As shown, the c|T< >a|A % progressively increases with the stage of cancer. FIG. 35B shows an ROC curve using c|T< >a|A to distinguish between eHCC and iHCC. FIG. 35C shows an ROC curve using c|T< >a|A to distinguish between iHCC and aHCC. FIG. 35D shows an ROC curve using c|T< >a|A to distinguish between eHCC and aHCC, with an AUC of 1 achieved.

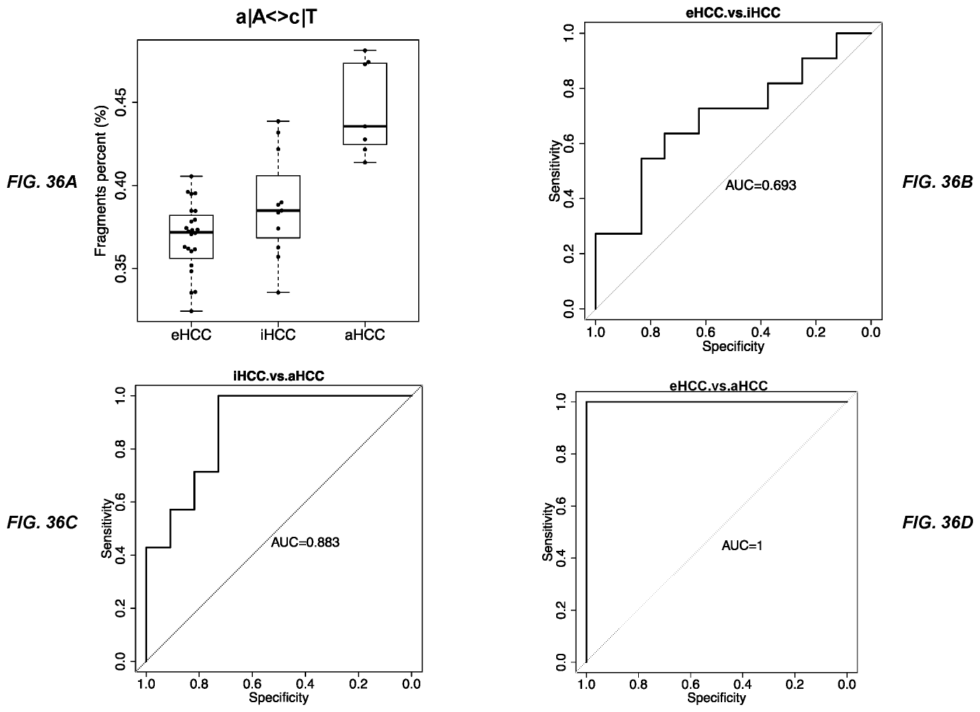

FIGS. 36A-36D provide performance results for the best performing biterminal −1 and +1 position motifs in distinguishing early vs advanced HCC. FIG. 36A shows a box plot of a|A< >c|T % for the three HCC stages. As shown, the a|A< >c|T % progressively increases with the stage of cancer. FIG. 36B shows an ROC curve using a|A< >c|T to distinguish between eHCC and iHCC. FIG. 36C shows an ROC curve using a|A< >c|T to distinguish between iHCC and aHCC. FIG. 36D shows an ROC curve using a|A< >c|T to distinguish between eHCC and aHCC, with an AUC of 1 achieved.

B. SLE

Some embodiments can also classify levels of an auto-immune disorder as the pathology (e.g., systemic lupus erythematosus, SLE). Bisulfite sequencing was performed for 34 samples (10 controls, 10 inactive SLE, 14 active SLE). The SLE activity was determined by SLEDAI (Systemic Lupus Erythematosus Disease Activity Index).

1. +1 End Motif Pairs

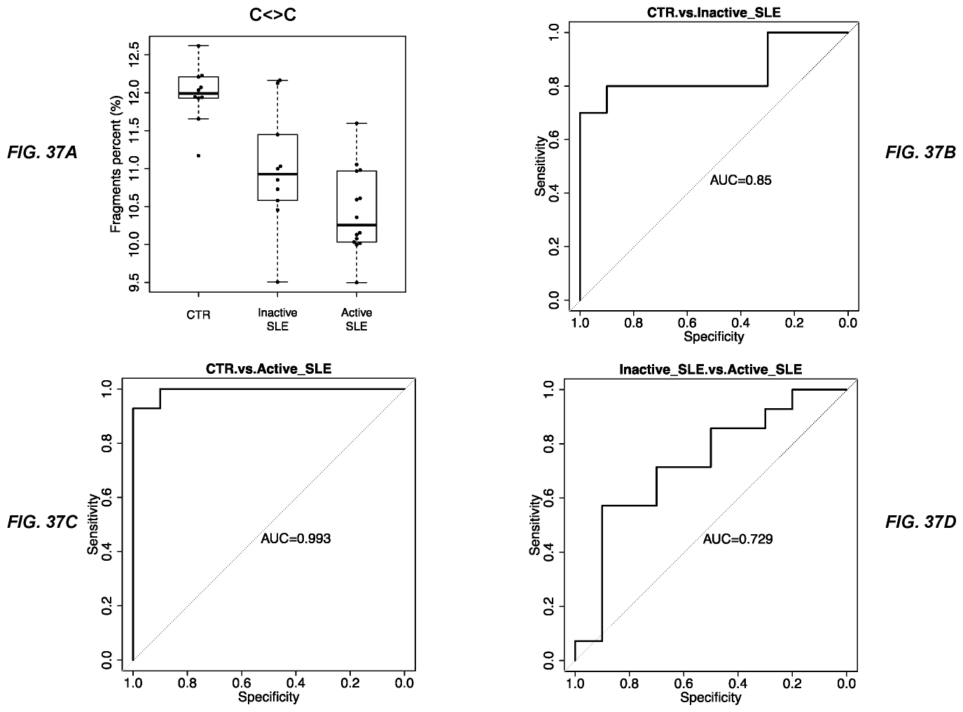

FIGS. 37A-37D show performance for C< >C in distinguishing controls, inactive SLE, and active SLE according to embodiments of the present disclosure. The fragment type C< >C is the best biterminal+1 position motifs for differentiating control vs active SLE.

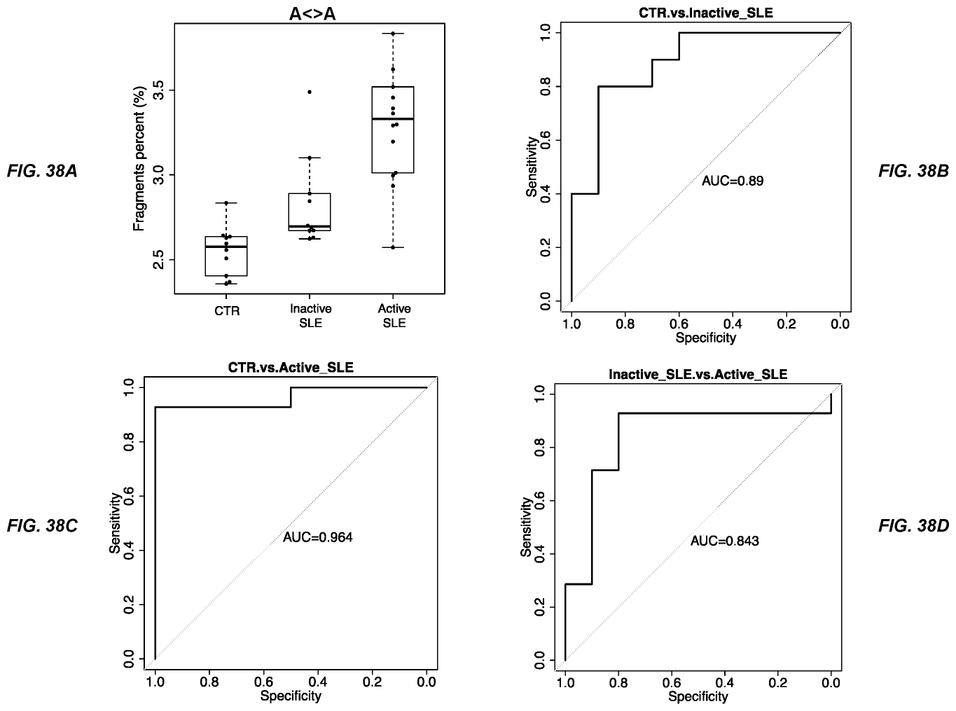

FIGS. 38A-38D show performance for A< >A in distinguishing controls, inactive SLE, and active SLE according to embodiments of the present disclosure. The fragment type A< >A is the best biterminal+1 position motifs for differentiating control vs inactive SLE and for inactive SLE vs active SLE.

2. +2 End Motif Pairs

The best performing biterminal+2 fragment types are provided in table 2 for distinguishing controls, inactive SLE, and active SLE. Box plots and ROC curves for certain fragment types are provided as well.

TABLE 2
End motif pairs with the highest AUC in distinguishing control vs
inactive SLE, control vs active SLE, inactive SLE vs active SLE.
The numbers represent the area-under-the-curve
(AUC) for Receiver Operating Characteristics Curve analysis.

| | Control vs | Control vs | Inactive SLE vs |
|---|---|---|---|
| Biterminal +2 | Inactive | Active | Active |
| Motif | SLE AUC | SLE AUC | SLE AUC |
| CC <> CC | 0.93 | 1 | 0.721 |
| TG <> CC | 0.92 | 1 | 0.9 |
| CC <> TG | 0.91 | 1 | 0.893 |
| CA <> CC | 0.9 | 1 | 0.789 |
| CC <> CA | 0.9 | 1 | 0.796 |
| AA <> CA | 0.71 | 1 | 0.886 |
| CA <> AA | 0.71 | 1 | 0.886 |
| CG <> CC | 0.89 | 1 | 0.775 |
| CC <> CG | 0.89 | 1 | 0.786 |
| GA <> CT | 0.8 | 1 | 0.868 |
| CT <> GA | 0.8 | 1 | 0.868 |
| AG <> AA | 0.9 | 1 | 0.871 |
| AA <> AG | 0.9 | 1 | 0.864 |
| GT <> CT | 0.895 | 1 | 0.882 |
| CT <> GT | 0.9 | 1 | 0.879 |
| GT <> TC | 0.78 | 1 | 0.893 |
| TC <> GT | 0.77 | 1 | 0.886 |
| GT <> TG | 0.95 | 0.979 | 0.629 |
| TG <> GG | 0.61 | 0.936 | 0.929 |

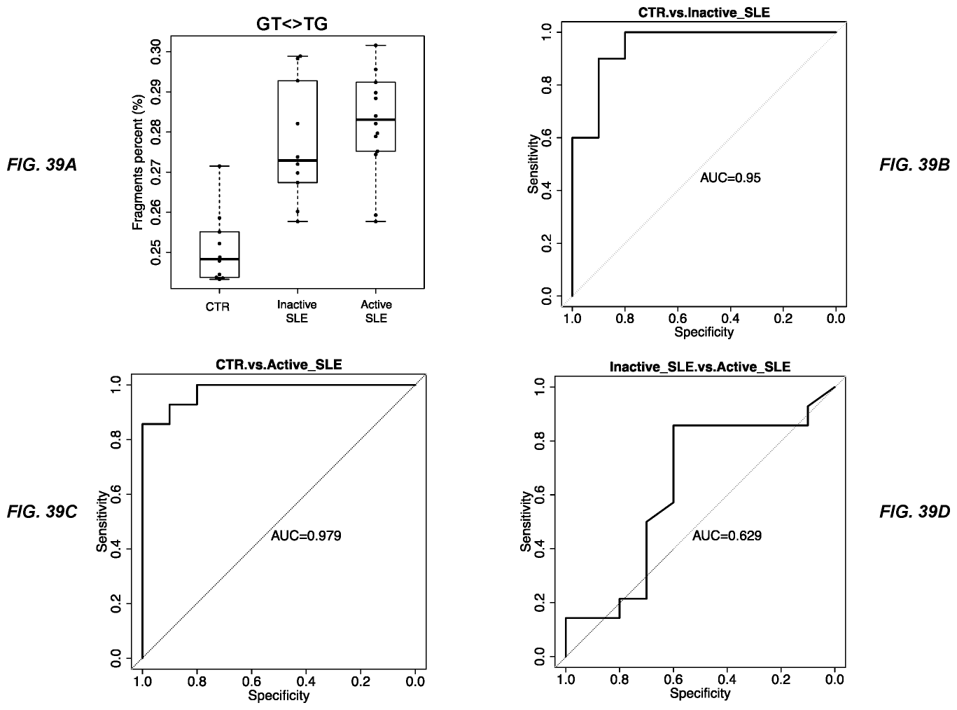

FIGS. 39A-39D show performance for GT< >TG in distinguishing controls, inactive SLE, and active SLE according to embodiments of the present disclosure. The fragment type GT<TG is the best biterminal+2 position motifs for differentiating control vs inactive SLE. As one can see, FIG. 39A shows a good separation between control (CTR) and inactive SLE, which results in an AUC of 0.95 for distinguishing between CTR and inactive SLE.

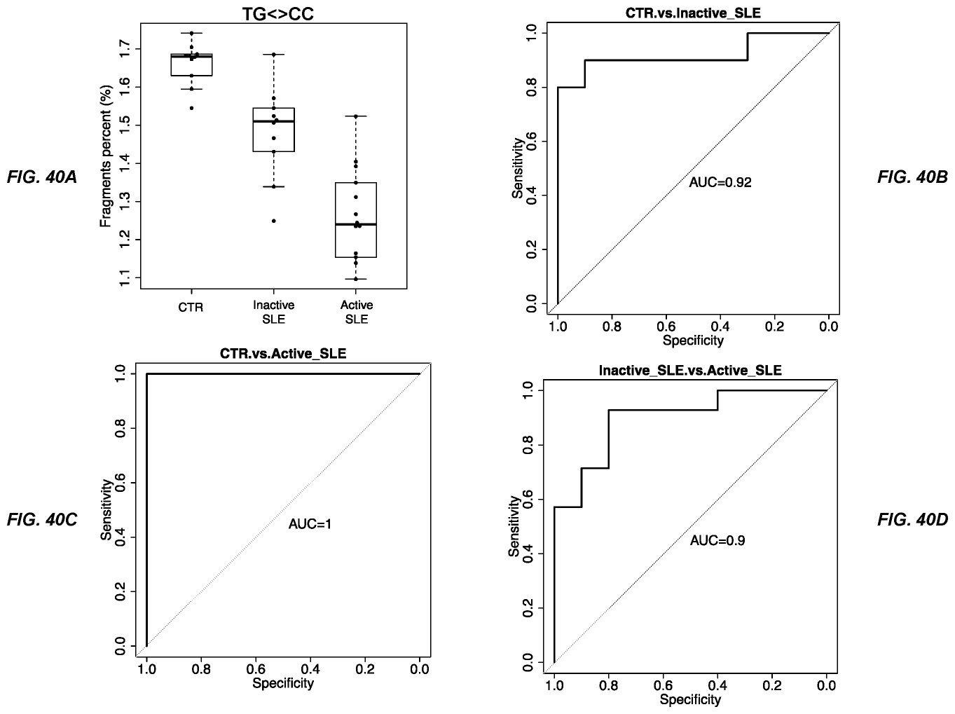

FIGS. 40A-40D show performance for TG< >CC in distinguishing controls, inactive SLE, and active SLE according to embodiments of the present disclosure. The fragment type TG<CC is tied for the best biterminal+2 position motifs for differentiating control vs active SLE. As one can see, FIG. 40A shows a good separation among all three classifications, and has a 100% accuracy between CTR and active SLE.

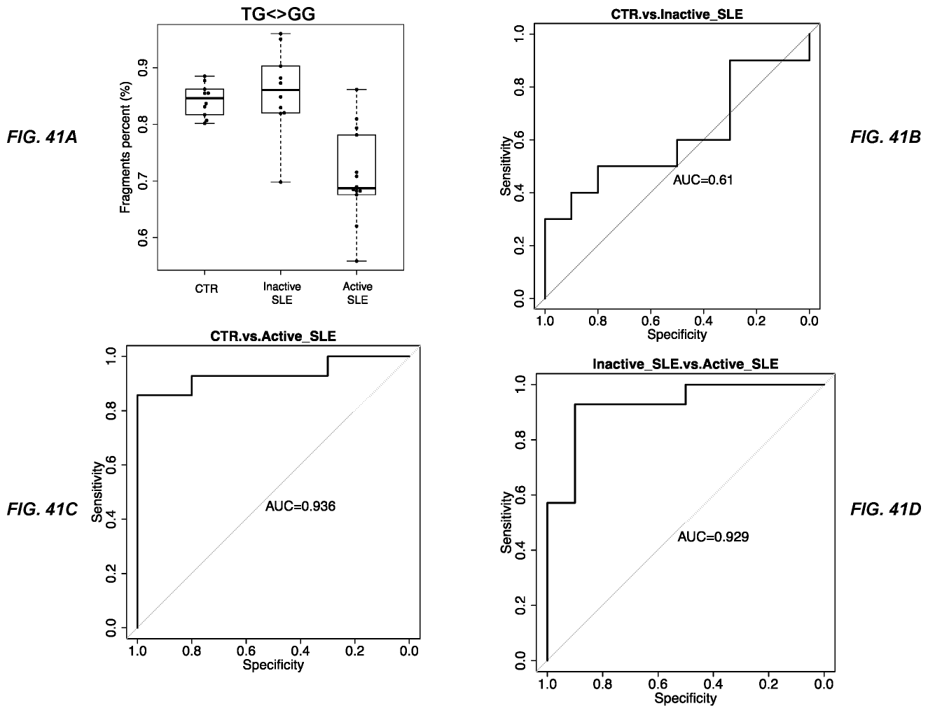

FIGS. 41A-41D show performance for TG< >GG in distinguishing controls, inactive SLE, and active SLE according to embodiments of the present disclosure. The fragment type TG<GG is the best biterminal+2 position motifs for differentiating inactive SLE vs active SLE. As one can see, FIG. 41A shows CTR and inactive SLE with similar median values. However, FIG. 41A shows a good separation between inactive SLE and active SLE, which results in an AUC of 0.929 for distinguishing between inactive SLE and active SLE.

3. −1 and +1 End Motif Pairs

The best performing biterminal −1 and +1 fragment types are provided in table 3 for distinguishing controls, inactive SLE, and active SLE. Box plots and ROC curves for certain fragment types are provided as well.

TABLE 3
−1 and +1 end motif pairs with the highest AUC in distinguishing
control vs inactive SLE, control vs active SLE, inactive SLE vs
active SLE. The numbers represent the area-under-the-curve
(AUC) for Receiver Operating Characteristics Curve analysis.

| | Control vs | Control vs | Inactive SLE vs |
|---|---|---|---|
| Biterminal +2 | Inactive | Active | Active |
| t\|C <> t\|C | 0.79 | 1 | 0.857 |
| t\|C <> a\|C | 0.79 | 1 | 0.857 |
| a\|C <> t\|C | 0.79 | 1 | 0.857 |
| a\|A <> c\|A | 0.94 | 1 | 0.764 |
| c\|A <> a\|A | 0.95 | 1 | 0.75 |
| g\|C <> g\|C | 0.86 | 0.757 | 0.921 |

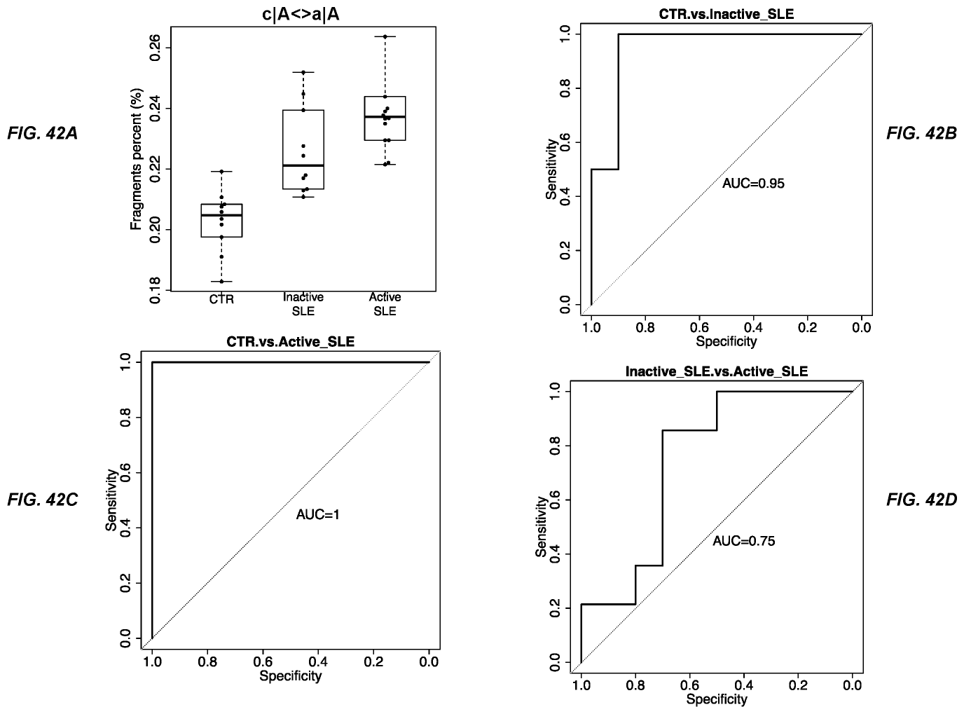

FIGS. 42A-42D show performance for c|A< >a|A in distinguishing controls, inactive SLE, and active SLE according to embodiments of the present disclosure. The fragment type c|A< >a|A is the best biterminal −1 and +1 position motifs for differentiating control vs inactive SLE. As one can see, FIG. 42A shows a good separation between control (CTR) and inactive SLE, which results in an AUC of 0.95 (FIG. 42B) for distinguishing between CTR and inactive SLE. The fragment type c|A< >a|A is also tied for the best biterminal −1 and +1 position motifs for differentiating control vs active SLE. As one can see, FIG. 42C shows 100% accuracy between CTR and active SLE.

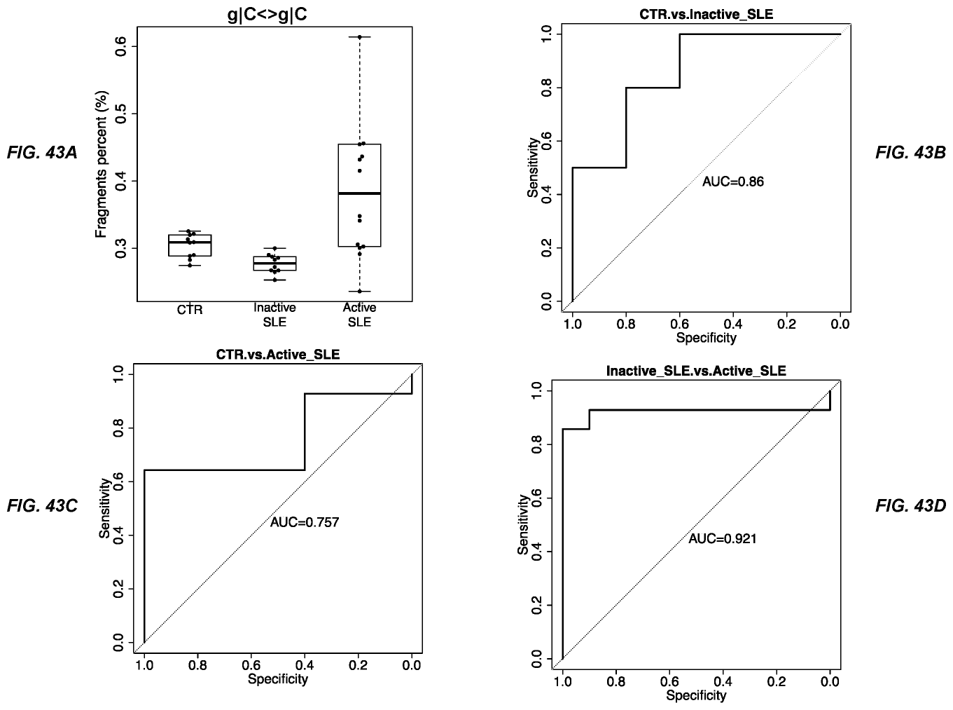

FIGS. 43A-43D show performance for g|C< >g|C in distinguishing controls, inactive SLE, and active SLE according to embodiments of the present disclosure. The fragment type g|C< >g|C is the best biterminal −1 and +1 position motifs for differentiating inactive SLE vs active SLE. As one can see, FIG. 43A shows a good separation between inactive SLE and active SLE, which results in an AUC of 0.921 (FIG. 43D) for distinguishing between inactive SLE and active SLE.

Different fragment types can be used in combination to determine which of the classifications is correct. For example, a best performing fragment type (or one with sufficient accuracy) can be used for each of the three pairwise comparisons, e.g., a comparison to a reference value that discriminates between the two classifications for that comparison. Then, if two of the three comparisons provide the same classification, then that classification can be used. As another example, only two comparisons are needed. For example, a Control vs Inactive comparison can be first performed. Then, if the first classification is Control, then a Control vs Active comparison can be performed to confirm the Control classification. If the first classification is Inactive, then an Inactive vs Active comparison can be performed to confirm the Inactive classification. If the second classification is different than the first classification, then the third pairwise comparison can be performed to determine if the third classification matches second classification. Other examples can use decision trees, SVMS, or other machine learning techniques.

### V. Effect of Sequencing Depth on Accuracy

In this section, we discuss effects of sequencing depth on accuracy. The analysis in section II used a median number of paired-reads of 215 million (range: 97-1,681 million). However, fewer reads may provide sufficient accuracy, thereby enabling less sequencing and smaller samples.

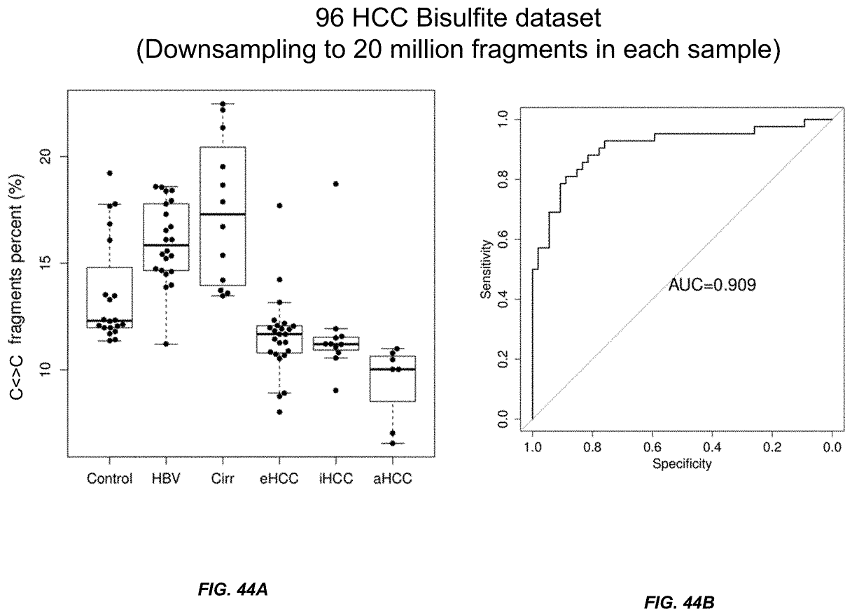

FIGS. 44A-44B show the performance for C< >C fragments in distinguishing between non-cancer and HCC using fewer fragments (20 million fragments) in each sample according to embodiments of the present disclosure. The box plot in FIG. 44A is similar to the box plot in FIG. 7D, even though fewer DNA fragments were analyzed, and the ROC curve in FIG. 44B is similar to the ROC curve in FIG. 7C. Thus, FIGS. 44A-44B show that even with a shallower sequencing depth, good accuracy can still be obtained. For example, an AUC of 0.909 is achieved with 20 million fragments.

We performed a further investigation of the performance using different numbers of fragments. We increased the number of reads, which increased the performance of the test, e.g., as measured by AUC. We illustrate the performance of biterminal CC< >CC % in samples with low sequencing depth by performing downsampling analysis.

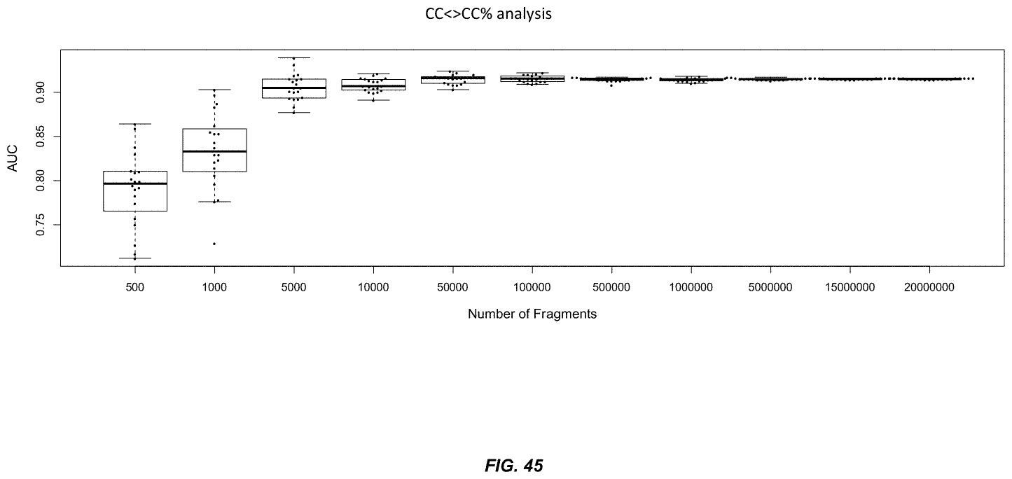

FIG. 45 is a graph depicting the AUC achievable using CC< >CC fragments as a function of the total number of fragments sequenced estimated through a downsampling analysis according to embodiments of the present disclosure. From the sequenced fragments of each sample, a smaller subset of reads were randomly sampled, and the CC< >CC % analysis was done to obtain an AUC. For each smaller subset of reads, random sampling was done 20 times. Progressively smaller subsets of reads were sampled to illustrate the lower limit of sequencing reads required for CC< >CC % analysis.

In FIG. 45, with 5,000 fragments sequenced, the median AUC achieved is above 0.9. With increasing number of fragments sequenced, the variation in the AUC achieved with CC< >CC % analysis is reduced. Accordingly, already at 5,000 fragments, embodiments can discriminate between different classifications for cancer with reasonable accuracy. As mentioned above, a sample of less than 1 microliter can be used, and even around one nanoliter for 5,000 fragments. Further, the time and cost can be relatively low when sequencing 5,000 fragments, e.g., compared to the typical 5 million fragments sequenced in non-invasive prenatal aneuploidy tests.

### VI. Pathology Screening Using End Motif Pairs

In accordance with the description above, some embodiments may provide a method of analyzing a biological sample of a subject to determine a level of pathology, where the biological sample includes cell-free DNA, e.g., as exists in plasma or serum. Example pathologies include liver pathologies (e.g., chronic hepatitis due to HBV or cirrhosis, or HCC), as well as other pathologies of other organs, such as other cancers. Another example includes auto-immune disorders, such as SLE.

A. Method for Pathology Screening

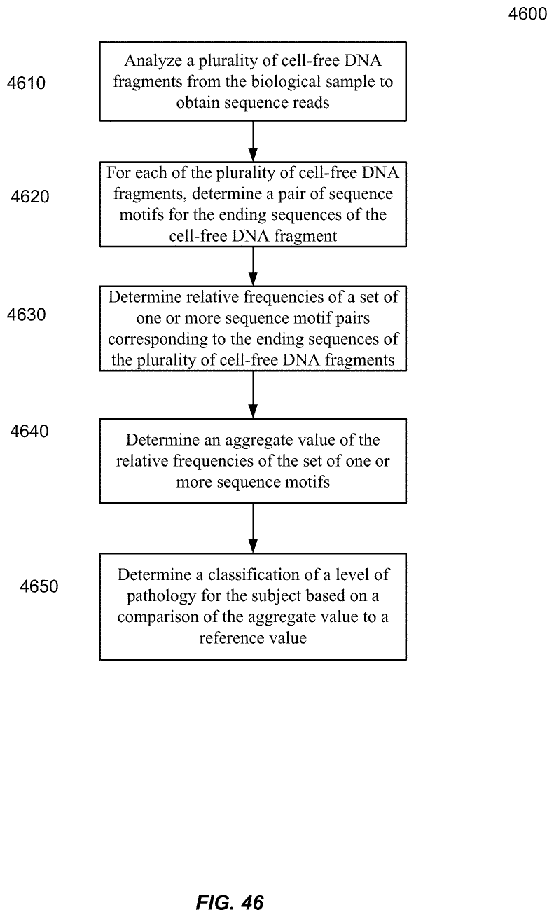

FIG. 46 is a flowchart illustrating a method for determining a level of pathology using end motif pairs of cell-free DNA (cfDNA) fragments according to embodiments of the present disclosure. The level of pathology can be determined from a biological sample of a subject, where the biological sample includes a mixture of cfDNA fragments derived from normal tissue (i.e., cells not affected by the pathology) and potentially cfDNA fragments derived from diseased tissue that is affected by the pathology (e.g., when the pathology exists in the subject). The cfDNA fragment derived from the diseased tissue can be considered clinically-relevant DNA, and the normal tissue can be considered other DNA. Aspects of method 4600 and any other methods described herein may be performed by a computer system.

At block 4610, a plurality of cell-free DNA fragments from the biological sample is analyzed to obtain sequence reads. The sequence reads include ending sequences corresponding to ends of the plurality of cell-free DNA fragments. As examples, the sequence reads can be obtained using sequencing or probe-based techniques, either of which may including enriching, e.g., via amplification or capture probes.

The sequencing may be performed in a variety of ways, e.g., using massively parallel sequencing or next-generation sequencing, using single molecule sequencing, and/or using double- or single-stranded DNA sequencing library preparation protocols. The skilled person will appreciate the variety of sequencing techniques that may be used. As part of the sequencing, it is possible that some of the sequence reads may correspond to cellular nucleic acids. The sequencing may be targeted sequencing as described herein. For example, biological sample can be enriched for DNA fragments from a particular region. The enriching can include using capture probes that bind to a portion of, or an entire genome, e.g., as defined by a reference genome.

A statistically significant number of cell-free DNA molecules can be analyzed so as to provide an accurate determination of the fractional concentration. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed.

At block 4620, for each of the plurality of cell-free DNA fragments, a pair of sequence motifs is determined for the ending sequences of the cell-free DNA fragment. These end motif pairs can correspond to the different types of fragments described herein, e.g., for 1-mers, 2-mers, etc. An end motif pair can include K base positions (e.g., 1, 2, 3, 4, 5, 6, etc.) at one end and M base positions (e.g., 1, 2, 3, 4, 5, 6, etc.) at the other end for a total of K+M=N bases. A particular end motif can include including position(s) on the other side of a cutting site, as described herein. Accordingly, the set of one or more sequence motif pairs can include N base positions, composed of K bases at one end and M bases at the other end. As examples, an end motif pair can be determined by analyzing the sequences at the end of the DNA fragment (e.g., using a pair of sequence reads or a single sequence read of the entire fragment), correlating a signal(s) with a particular motif pair (e.g., when a probe(s) is used), and/or aligning the sequence read(s) to a reference genome, e.g., as described in technique 160 of FIG. 1 or in FIG. 4C.

For example, after sequencing by a sequencing device, the sequence reads may be received by a computer system, which may be communicably coupled to a sequencing device that performed the sequencing, e.g., via wired or wireless communications or via a detachable memory device. In some implementations, one or more sequence reads that include both ends of the nucleic acid fragment can be received. The location of a DNA molecule can be determined by mapping (aligning) the one or more sequence reads of the DNA molecule to respective parts of the human genome, e.g., to specific regions. In other embodiments, a particular probe (e.g., following PCR or other amplification) can indicate a location or a particular end motif, such as via a particular fluorescent color. Particular combination of two colors (examples of signals) can indicate a particular pair of end motifs. The identification can be that the cell-free DNA molecule corresponds to one of a set of sequence motif pairs.

At block 4630, one or more relative frequencies of a set of one or more sequence motif pairs corresponding to the ending sequences of the plurality of cell-free DNA fragments are determined. A relative frequency of a sequence motif pair can provide a proportion of the plurality of cell-free DNA fragments that have a pair of ending sequences corresponding to the sequence motif pair. Examples of relative frequencies are described throughout the disclosure.

The set of one or more sequence motif pairs can be identified using a reference (training) set of reference (training) samples having known levels of the pathology. An example set of reference samples is the 96 samples used in section II, which can be used to determine specific end motif pairs that are used to train a model, e.g., determining reference value(s) that satisfy sensitivity and specificity criteria. Particular end motif pairs can be selected on the basis of the differences for discriminating between classifications (e.g., to select the end motif pairs with the highest absolute or percentage difference). For example, the set of one or more sequence motif pairs can be a top L sequence motif pairs with a largest difference between two classified reference samples, e.g., the motifs that show a largest positive difference (e.g., top 1, 2, 3, etc. or other number) or show a largest negative difference. L can be an integer equal to or greater than one. Using the top sequence motif pairs (i.e., end motif pairs) is an example of using a subset of all possible combinations of a particular fragment type.

All or a subset of combinations of sequence motif pairs of a particular type can be used, or even combinations across various types (all or a subset). Thus, the set of one or more sequence motif pairs can include all combinations of N bases (K at one end and M at the other end), where N is an integer equal to or greater than two. As another example, the set of one or more sequence motif pairs can be a top J most frequent sequence motif pairs occurring in one or more reference samples, with J being an integer equal to or greater than one.

At block 4640, an aggregate value of the relative frequencies of the set of one or more sequence motif pairs is determined. Example aggregate values are described throughout the disclosure, e.g., including just one relative frequency itself, a sum of relative frequencies, and a distance between reference data point (reference pattern determined from reference samples) and a multidimensional data point corresponding to a vector of relative frequencies for a set of K end motif pairs. Accordingly, when the set of one or more sequence motif pairs includes a plurality of sequence motifs, the aggregate value can include a sum of the relative frequencies of the set. The sum can be a weighted sum, e.g., relative frequencies that provide higher discrimination (e.g., as determined by AUC) can be weighted higher.

As another example, the aggregate value can include a difference (e.g., a distance) of the multidimensional data point from a reference pattern (data point) of relative frequencies. Accordingly, determining the aggregate value of the plurality of relative frequencies can includes determining a difference between each of the plurality of relative frequencies and a reference frequency of a reference pattern, with the aggregate value including a sum of the differences. The reference frequencies of the reference pattern can be determined from one or more reference samples having a known classification.

The distance can be a Euclidean distance or be weighted for the different dimensions, e.g., for the dimension of an end motif that provides higher discrimination. This distance can be used in clustering, support vector machine (SVMs), or other machine learning models. The reference pattern can be established from the training set of reference samples. The reference pattern for a given classification for the level of pathology can be determined as a centroid of a cluster of data points having that classification. The aggregate value can be derived from such a distance, e.g., a probability determined from the difference or a final or intermediate output in a machine learning model (e.g., an intermediate or final layer in a neural network). Such a value can be compared to a cutoff (reference value in a following block) between two classifications or compared to a representative value of a given classification. In various implementations, the machine learning model uses clustering, neural networks, SVMs, or logistic regression.

At block 4650, a classification of a level of pathology for the subject is determined based on a comparison of the aggregate value to a reference value. As examples, the levels can be no pathology (e.g., cancer), early stage, intermediate stage, or advanced stage. The classification can then select one of the levels. Accordingly, the classification can be determined from a plurality of levels of pathology that include a plurality of stages of pathology (e.g., cancer or of SLE). The reference value can be determined from the reference samples, e.g., using the ROC curves described herein. As examples when the pathology is cancer, the cancer can be hepatocellular carcinoma, lung cancer, breast cancer, gastric cancer, glioblastoma multiforme, pancreatic cancer, colorectal cancer, nasopharyngeal carcinoma, and head and neck squamous cell carcinoma, or other cancer mentioned herein. As the stages of a disease (e.g., cancer) can be associated with outcome, prognosis, remission, survival, or response to treatment, embodiments have valuable utility in healthcare.

In some embodiments, the cell-free DNA are filtered using one or more criteria to identify the plurality of cell-free DNA fragments. Examples of filtering are provided herein. For example, the filtering can be based on a methylation (density or whether a particular site is methylated), size, or a region from which a DNA fragment is derived. The cell-free DNA can be filtered for DNA fragments from open chromatin regions of a particular tissue.

As described above, combining the relative frequencies of more than one end motif pair to determine an aggregate value can achieve better performance. Additionally or alternatively, the classifications for different sets of one or more end motif pairs can be combined, e.g., in an ensemble technique. Example ensemble techniques include voting (e.g., majority voting, equal weight for voting as may be done in bagging, and weighting by likelihood of classification in a training set or in a population), averaging, and boosting.

In some embodiments, a first set of one or more end motif pairs can be used to determine a first classification, e.g., whether the pathology exists. For instance, C< >C can be used in a first pass that determine whether cancer exists. Then, blocks 4630-4650 can be repeated for a second set of one or more end motif pairs to differentiate between different stages of the pathology (e.g., cancer). For instance, A< >T can be used to differentiate between early, intermediate, and advanced stages of cancer. Accordingly, one or more one or more additional relative frequencies of a set of one or more additional sequence motif pairs corresponding to the ending sequences of the plurality of cell-free DNA fragments can be determined. And an additional aggregate value of the one or more additional relative frequencies of the set of one or more additional sequence motif pairs can be determined. A stage of the cancer for the subject can be determined based on a comparison of the additional aggregate value to an additional reference value. Examples for differentiating between stages of cancer are provided in section IV.A.

Multiple classifications can be performed for multiple sets of sequence motif pair(s), with each set providing a classification. These classifications can be combined (e.g., in an ensemble technique). Accordingly, the classification in block 4650 can be a first classification, and one or more additional classifications can be determined for one or more additional sets of sequence motif pairs. A final classification can then be determined using the first classification and one or more additional classifications, e.g., via a majority voting or a probability for a given classification can be determined from the various classifications.

Additionally, such biterminal analysis may be combined with other classifications, e.g., copy number aberrations, methylation signatures, or sequence mutations to improve performance. Such classifications can be combined in an ensemble technique.

B. Comparison with Other Techniques

Other works have also analyzed cfDNA to distinguish HCC and non-HCC. Jiang et al. used high depth sequencing of the plasma of an HCC patient to identify tumor-associated preferred end coordinates (9). A ratio of the tumor-associated to non-tumor-associated preferred ends was used to discriminate between non-HCC and HCC with an AUC of 0.88. The work by Jiang et al. is different from method 4600 in several ways: 1) they required high depth sequencing of the cfDNA of an HCC patient and an HBV carrier to obtain specific tumor and non-tumor associated genomic coordinates, 2) alignment of fragments back to reference genomic coordinates is required, and 3) they counted either end of a fragment aligning to the specific genomic coordinate as an end.

Another technique can use the 4-mer motif at the 5′ end to distinguish between cancer and non-cancer. The 4-mer motif frequencies can be calculated by considering separately the 5′ ends of each read of a fragment (two for each fragment). As examples, a particular motif can be used, or a derived entropy score from the 4-mer motifs, referred to as the motif diversity score (MDS), can be used to distinguish HCC and non-HCC with an AUC of 0.856. MDS is an example of a variance. To analyze the distribution of frequencies of motifs (e.g. for a total of 256 motifs for a 4-mer), one definition of MDS uses the following equation:

$$M D S = {\sum }_{i = 1}^{256} - {P}_{i} * log ( {P}_{i} )$$

where Pᵢ is the frequency of a particular motif; a higher entropy value indicates a higher diversity (i.e. a higher degree of randomness).

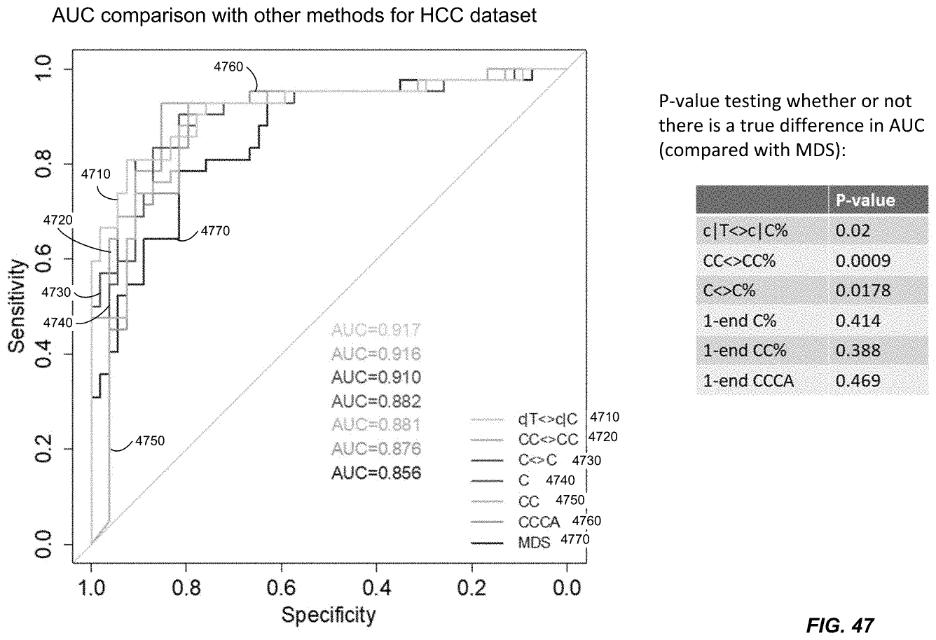

FIG. 47 shows multiple ROC curves from different methods of analysis on the same non-HCC and HCC dataset according to embodiments of the present disclosure. The AUC of each method is also shown. The P-value tests for a true difference in the various AUCs compared with MDS. The dataset is the same as used in section II.

Each line in the box plot corresponds to a different technique, e.g., a different motif, whether both ends are used or just one end, and MDS. Line 4710 corresponds to c|T< >c|C. Line 4720 corresponds to CC< >CC. Line 4730 corresponds to C< >C. Line 4740 corresponds to a C at one end. Line 4750 corresponds to a CC at one end. Line 4760 corresponds to a CCCA at one end. Line 4770 corresponds to MDS.

In comparison with MDS and using each end separately for analysis (denoted as 1-end analysis), biterminal analysis using a relative amount of one or more types (fragments with a specified set of end motif pairs) performs better in the HCC dataset. The AUC for c|T< >c|C % is 0.917; the AUC for CC< >CC % is 0.916; and the AUC for C< >C % is 0.910. The AUC for 1-end analysis of C % is 0.882; CC % is 0.881%; CCCA % is 0.876; and MDS is 0.856. The AUCs achieved from c|T< >c|C %, CC< >CC % and C< >C % analysis are significantly different from the AUC of MDS (p-value 0.02, 0.0009 and 0.0178, respectively).

A comparison was also made between the biterminal analysis and MDS and 1-end analysis in other types of cancer.

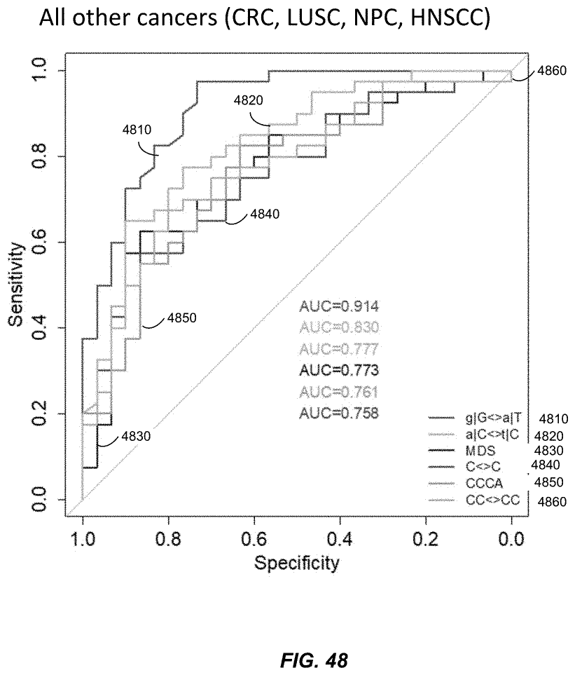

FIGS. 48-50B show multiple ROC curves from different methods of analysis of a data set with 30 controls and 40 other cancers with CRC, LUSC, NPC, and HNSCC according to embodiments of the present disclosure. The AUC of each method is also shown. The data set is the same as used in section III.

FIG. 48 shows the performance for collectively distinguishing cancer from non-cancer for various methods. Line 4810 corresponds to g|G< >a|T. Line 4820 corresponds to a|C< >t|C. Line 4830 corresponds to MDS. Line 4840 corresponds to C< >C. Line 4850 corresponds to a CCCA at one end. Line 4860 corresponds to CC< >CC. In this dataset with the 40 other cancers, g|G< >a|T and a|C< >t|C fragment % are example fragment types that have good performance with an AUC of 0.914 and 0.830, respectively. CC< >CC % has an AUC of 0.777 compared with 0.773 of MDS.

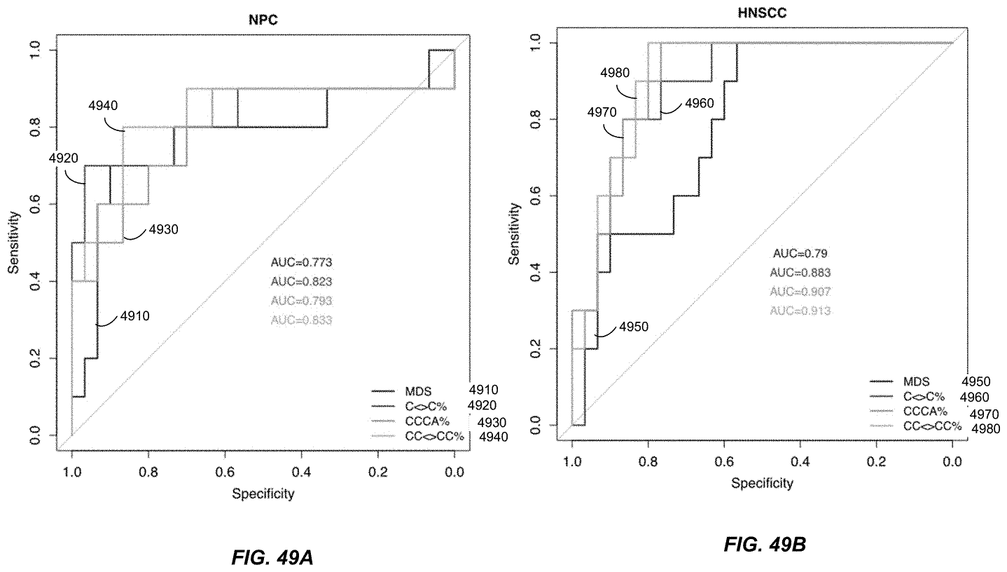

FIG. 49A shows the performance of various methods in distinguishing between controls and NPC according to embodiments of the present disclosure. Line 4910 corresponds to MDS. Line 4920 corresponds to C< >C. Line 4930 corresponds to CCCA at one end. Line 4940 corresponds to CC< >CC. For NPC, the ability to differentiate cancer and non-cancer using CC< >CC % has an AUC of 0.833.

FIG. 49B shows the performance of various methods in distinguishing between controls and HNSCC according to embodiments of the present disclosure. Line 4950 corresponds to MDS. Line 4960 corresponds to C< >C. Line 4970 corresponds to CCCA at one end. Line 4980 corresponds to CC< >CC. For HNSCC, the ability to differentiate cancer and non-cancer using CC< >CC % has an AUC of 0.913.

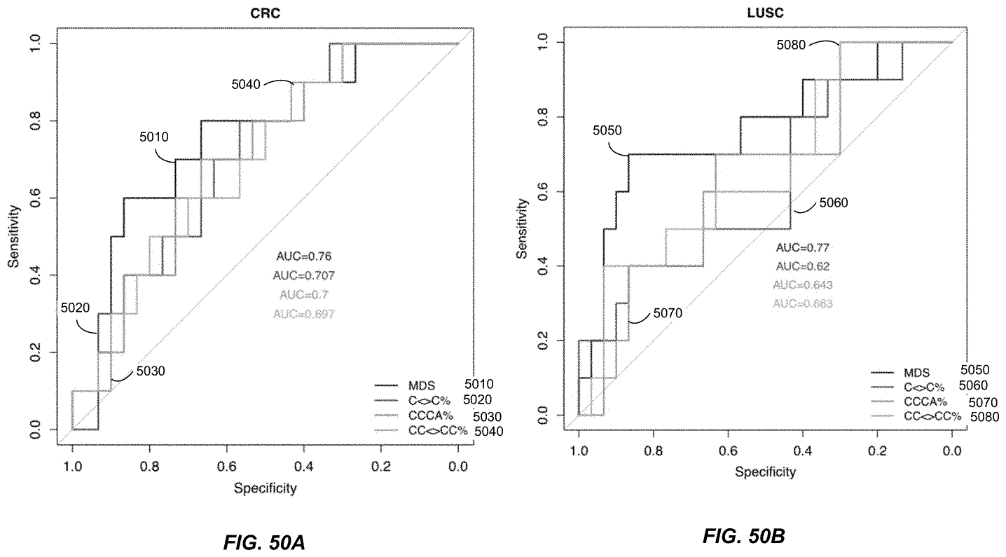

FIG. 50A shows the performance of various methods in distinguishing between controls and CRC according to embodiments of the present disclosure. Line 5010 corresponds to MDS. Line 5020 corresponds to C< >C. Line 5030 corresponds to CCCA at one end. Line 5040 corresponds to CC< >CC. For CRC, MDS performed the best with an AUC of 0.76.

FIG. 50B shows the performance of various methods in distinguishing between controls and LUSC according to embodiments of the present disclosure. Line 5050 corresponds to MDS. Line 5060 corresponds to C< >C. Line 5070 corresponds to CCCA at one end. Line 5080 corresponds to CC< >CC. For HNSCC, MDS performed the best with an AUC of 0.77. For CRC and LUSC, although differentiating cancer and non-cancer with CC< >CC % is possible, the AUC is less than that of MDS.

### VII. Fractional Concentration of Clinically-Relevant DNA

Another application of biterminal analysis is to distinguish between fetal and maternal DNA molecules. To assess the potential of biterminal analysis in distinguishing fetal and maternal molecules, we explore whether or not a difference in the fragment type percentages can be detected between known fetal and maternal molecules. Other embodiments may determine the fractional concentration of other clinically-relevant DNA, e.g., tumor and transplant.

A. Fetal Concentration

Fetal and maternal molecules were identified by using informative single nucleotide polymorphism (SNP) sites for which the mother is homozygous (AA) and the fetus is heterozygous (AB). The fetal-specific molecules carry the fetal-specific alleles (B). The molecules that carry the shared allele (A) represent the predominantly maternal-derived DNA molecules because the fetal DNA molecules generally account for only a minority of maternal plasma DNA.

Plasma and maternal buffy coat samples were obtained from pregnant women in the first trimester (12-14 weeks, n=10), second trimester (20-23 weeks, n=10), and third trimester (38-40 weeks, n=10). Samples of plasma and buffy coat were obtained from a total of 30 pregnant women (10 in each trimester). The maternal buffy coat and fetal samples were genotyped using a microarray platform (Human Omni2.5, Illumina), and the matched plasma DNA samples were sequenced. The skilled person will appreciate that other genotyping techniques and platforms may be used. A median of 195,331 informative SNPs (range: 146,428-202,800) was found where the mother was homozygous (AA) and the fetus was heterozygous (AB). A median of 103 million (range: 52-186 million) mapped paired-end reads was obtained for each case. The median fetal DNA fraction among those samples was 17.1% (range: 7.0%-46.8%).

1. Distinguishing Between Shared and Fetal Alleles

From this dataset, we tested the performance of biterminal analysis in distinguishing between fetal (Spec) and maternal (shared) molecules. The percentage of particular biterminal fragment types were analyzed to detect a difference in proportion between the DNA fragments having a shared allele (Shared) and the DNA fragments having a fetal-specific allele (Spec) at any of the informative sites. The percentage of any given fragment type for the shared alleles is determined using the total number of DNA fragments having a shared allele. The percentage of any given fragment type for the fetal-specific alleles is determined using the total number of DNA fragments having a fetal-specific SNP.

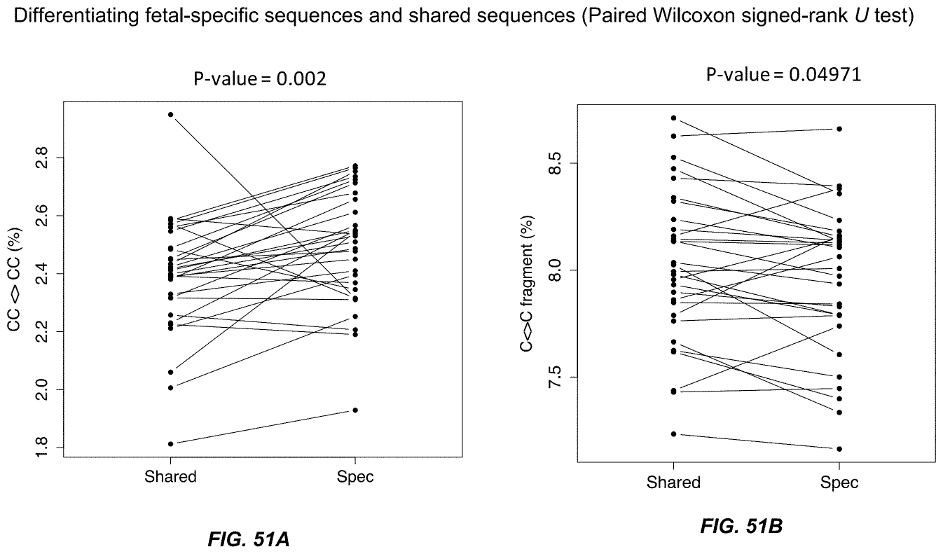

FIGS. 51A-51B show biterminal analysis in differentiating between fetal-specific molecules and shared molecules according to embodiments of the present disclosure. FIG. 51A shows the percentage of fragments having CC< >CC out of all of the fragments having a shared allele (Shared) and the percentage of fragments having CC< >CC out of all of the fragments having a fetal-specific allele (Spec). The lines connect the two data points of a same sample. As one can see, the percentage generally increases from the shared alleles to the fetal-specific alleles. FIG. 51B shows the percentage of fragments having C< >C out of all of the fragments having a shared allele (Shared) and the percentage of fragments having C< >C out of all of the fragments having a fetal-specific allele (Spec). The performance of CC< >CC is better than C< >C.

Using biterminal analysis with 2-mers, it is possible to distinguish between fetal-specific molecules and shared molecules. An embodiment using CC< >CC % is significantly higher in fetal-specific molecules than in shared molecules (Paired Wilcoxon signed-rank U test, P value=0.002). Accordingly, the existence of CC< >CC on a fragment indicates a higher likelihood that the fragment is from the fetus. Various embodiments can use such an increased likelihood to in various ways, such as to measure the concentration of fetal DNA fraction or filter out maternal DNA fragments, e.g., to enrich a sample of cfDNA fragments (sequence reads) for those that are of fetal origin. Such an enrichment can allow more accurate measurements, e.g., to detect aneuploidy or deletions/amplifications of a region.

2. Relationship with Fetal cfDNA Fraction

Given the higher likelihood of certain biterminal fragment types coming from fetal cells, embodiments can leverage such a relationship to measure the fetal DNA fraction in the cell-free DNA sample. For example, one can know the fetal DNA fraction for certain types of samples, e.g., where the fetus is male so that DNA fragments from the Y chromosome are fetal-specific or where a fetal-specific allele has been identified, as is described above. Then, once a correspondence is determined between fetal DNA fraction in known (calibration) samples and the proportion of a particular fragment type(s), a new measurement of the fragment type proportion in a new sample can provide the fetal DNA fraction.

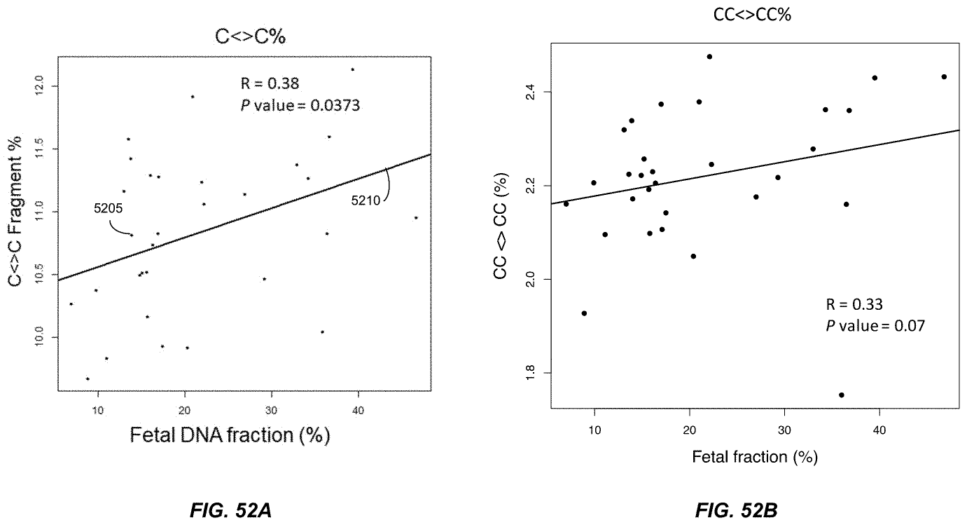

FIG. 52A shows a functional relationship between biterminal C< >C % and the fetal DNA fraction according to embodiments of the present disclosure. The horizontal axis is fetal DNA fraction, as measured using the fetal-specific SNPs described in the previous section. The vertical axis is the percentage of C< >C fragments in the sample. As one can see, the percentage of C< >C fragments is higher than 1/16, if each type of fragment was equally represented. Thus, a sufficient number of DNA fragment to make a statistically stable measurement can be made with a relatively small sample, compared to other fragment types that have a lower range of content. The C< >C % in FIG. 52A is determined using DNA fragments with shared and fetal-specific alleles.

The C< >C fragment percentage increases with the fetal DNA fraction, as signified by the positive slope of the calibration function, which is a linear function that is fit to the calibration data points 3605. Each of the calibration data points includes a measurement of the fetal DNA fraction (e.g., using a fetal-specific allele) and a measurement of C< >C fragment %, which is an example of a calibration value. If the C< >C fragment percentage is higher, then the fetal DNA fraction will be higher. Using the calibration function 3610, a measurement of about 11% for C< >C can be used to estimate the fetal DNA fraction to be about 30%. Accordingly, a biterminal analysis with C< >C % is a useful metric to estimate fetal fraction. The correlation of fetal fraction for C< >C % is R=0.38 (P value=0.0373).

FIG. 52B shows a functional relationship between biterminal CC< >CC % and the fetal DNA fraction according to embodiments of the present disclosure. Such a functional relationship can be used in a similar manner as FIG. 52A. The higher proportion of C< >C fragments may provide a more stable functional relationship to fetal DNA fraction, even though CC< >CC can provide better discrimination among DNA fragment. In this regard, there is an approximately 3-fold reduction in the amount of molecules when one compared the proportion of C< >C vs CC< >CC fragments.

A similar analysis can be performed for other types of clinically-relevant DNA, e.g., for tumor DNA or DNA from a transplanted organ.

B. Concentration of other clinically-relevant DNA

Clinically-relevant DNA can also include tumor DNA. Some embodiments can determine a tumor DNA concentration in a sample in a similar manner as the fetal concentration is determined above.

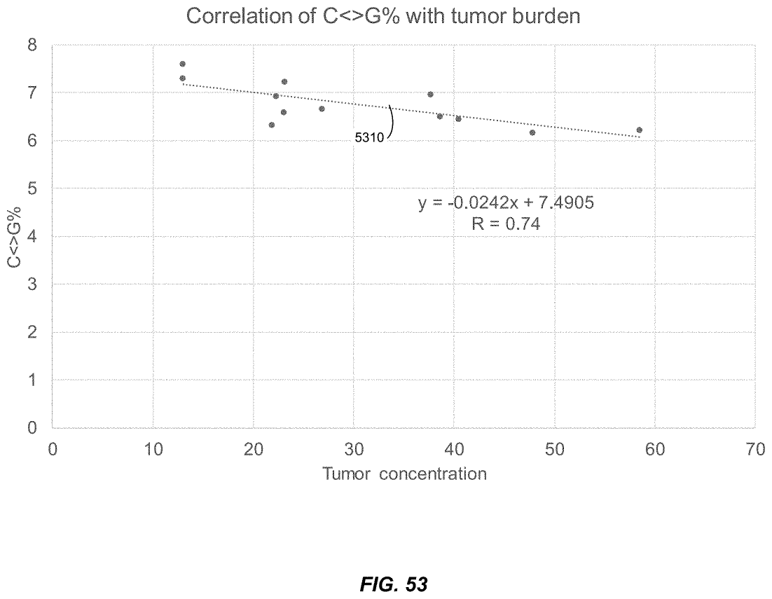

FIG. 53 shows the functional relationship between C< >G % and tumor concentration according to embodiments of the present disclosure. In the HCC samples, IchorCNA (Adalsteinsson et al, Nat Commun. 2017; 8: 1324) was used to independently estimate tumor concentration from copy number alterations (CNA). Of the HCC samples, only 12 samples had sufficient CNA for IchorCNA to estimate a tumor concentration. The biterminal 1-mer fragment percentage with the best correlation with IchorCNA tumor fraction is shown. As tumor concentration increases, C< >G % decreases. R value is 0.74. The dependence on tumor concentration is quite good. The calibration function is provided as a linear function in FIG. 53.

C. Distinguishing Transplant DNA and Host DNA

Clinically-relevant DNA can also include transplant DNA. Some embodiments can determine a transplant DNA concentration in a sample in a similar manner as the fetal and tumor concentration is determined above.

1. Liver

Biterminal end analysis was performed for 12 liver transplant cases. Donor-specific SNPs were used to identify liver-specific fragments. Fragment type percentages were compared between donor-specific fragments and fragments with shared SNPs. The five fragment types having the most significant differences are provided below. P values are provided by Wilcoxon signed-rank test.

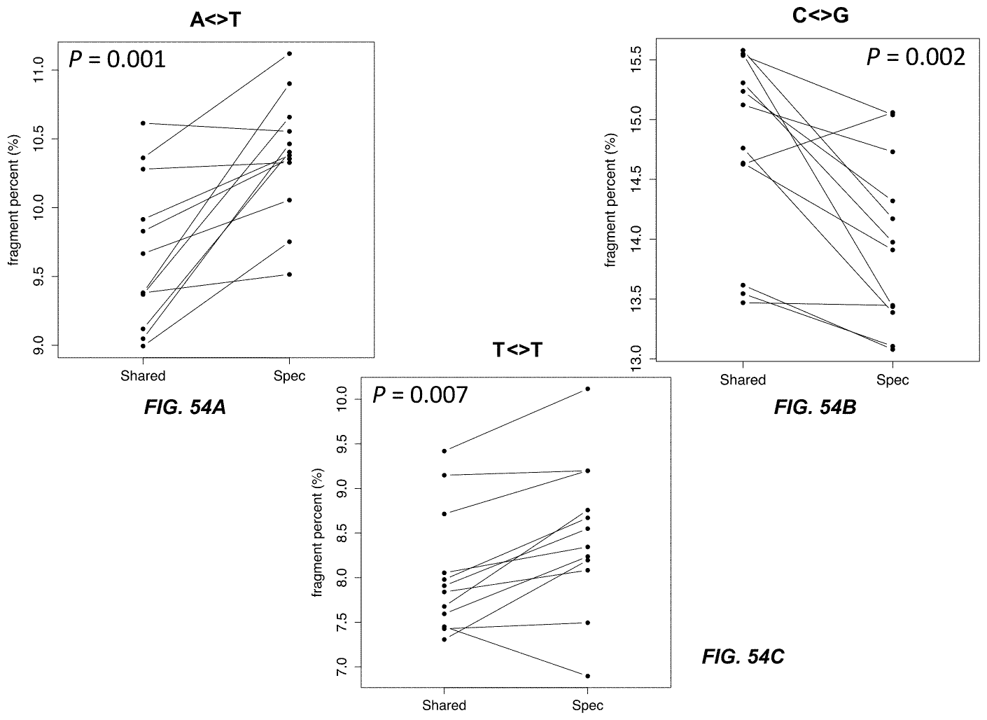

FIG. 54A shows the percentage of fragments having A< >T out of all of the fragments having a shared allele (Shared) and the percentage of fragments having A< >T out of all of the fragments having a donor-specific allele (Spec). As one can see, the percentage generally increases from the shared alleles to the donor-specific alleles. The statistical difference of P=0.001 (best in present data) between the two data sets shows a distinction between the A< >T % values for the two types of tissue: host and transplant.

FIG. 54B shows the percentage of fragments having C< >G out of all of the fragments having a shared allele (Shared) and the percentage of fragments having C< >G out of all of the fragments having a donor-specific allele (Spec). As one can see, the percentage generally decreases from the shared alleles to the donor-specific alleles. The statistical difference of P=0.002 between the two data sets shows a distinction between the C< >G % values for the two types of tissue: host and transplant.

FIG. 54C shows the percentage of fragments having T< >T out of all of the fragments having a shared allele (Shared) and the percentage of fragments having T< >T out of all of the fragments having a donor-specific allele (Spec). As one can see, the percentage generally increases from the shared alleles to the donor-specific alleles. The statistical difference of P=0.007 between the two data sets shows a distinction between the T< >T % values for the two types of tissue: host and transplant.

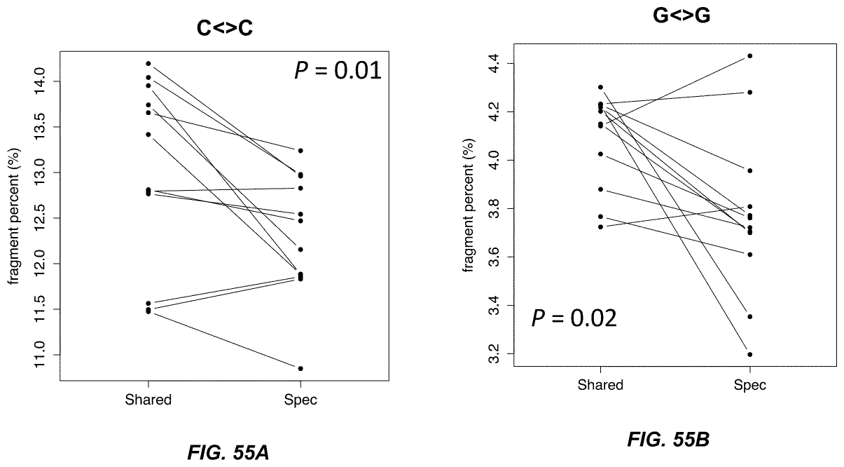

FIG. 55A shows the percentage of fragments having C< >C out of all of the fragments having a shared allele (Shared) and the percentage of fragments having C< >C out of all of the fragments having a donor-specific allele (Spec). As one can see, the percentage generally decreases from the shared alleles to the donor-specific alleles. The statistical difference of P=0.01 between the two data sets shows a distinction between the C< >C % values for the two types of tissue: host and transplant.

FIG. 55B shows the percentage of fragments having G< >G out of all of the fragments having a shared allele (Shared) and the percentage of fragments having G< >G out of all of the fragments having a donor-specific allele (Spec). As one can see, the percentage generally decreases from the shared alleles to the donor-specific alleles. The statistical difference of P=0.007 between the two data sets shows a distinction between the G< >G % values for the two types of tissue: host and transplant.

2. Kidney

Biterminal end analysis was performed in 12 kidney transplant cases. Fragment type percentages were compared between donor-specific fragments and fragments with shared SNPs. The two fragment types having the most significant differences are provided below. P values are provided by Wilcoxon signed-rank test.

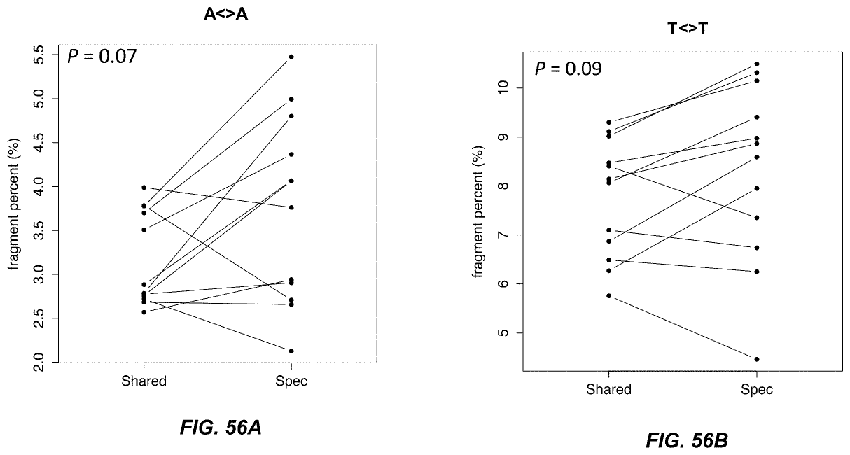

FIG. 56A shows the percentage of fragments having A< >A out of all of the fragments having a shared allele (Shared) and the percentage of fragments having A< >A out of all of the fragments having a donor-specific allele (Spec). As one can see, the percentage generally increases from the shared alleles to the donor-specific alleles. The statistical difference of P=0.07 between the two data sets shows a distinction between the A< >A % values for the two types of tissue: host and transplant.

FIG. 56B shows the percentage of fragments having T< >T out of all of the fragments having a shared allele (Shared) and the percentage of fragments having T< >T out of all of the fragments having a donor-specific allele (Spec). As one can see, the percentage generally increases from the shared alleles to the donor-specific alleles. The statistical difference of P=0.09 between the two data sets shows a distinction between the T< >T % values for the two types of tissue: host and transplant.

D. Method of Determining Concentration

In accordance with the description above, some embodiments may estimate a fractional concentration of clinically-relevant DNA (e.g., fetal or tumor DNA) in a biological sample of a subject, where the biological sample includes a mixture of the clinically-relevant DNA and other DNA that are cell-free. In other examples, a biological sample may not include the clinically-relevant DNA, and the estimated fractional concentration may indicate zero or a low percentage of the clinically-relevant DNA.

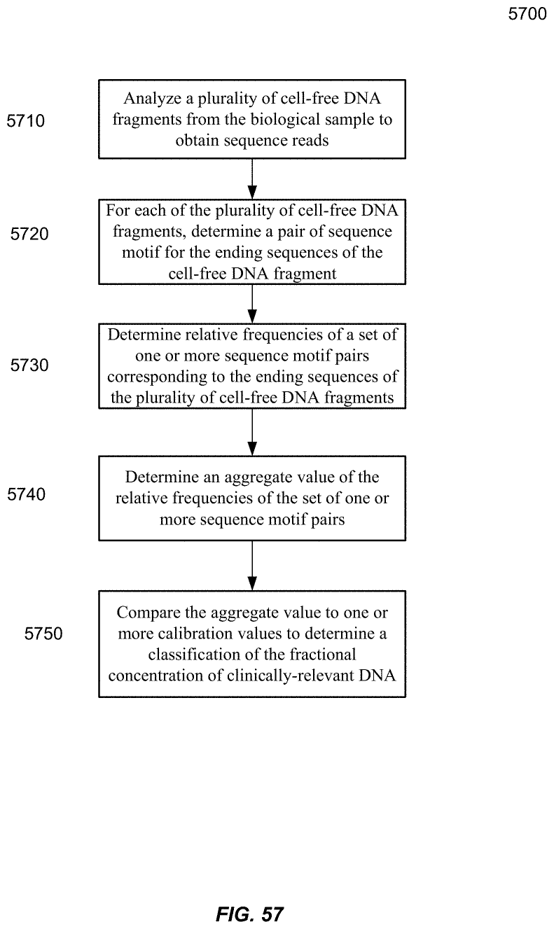

FIG. 57 is a flowchart illustrating a method 5700 of estimating a fractional concentration of clinically-relevant DNA in a biological sample of a subject according to embodiments of the present disclosure. Aspects of method 5700 and any other methods described herein may be performed by a computer system.

At block 5710, a plurality of cell-free DNA fragments from the biological sample are analyzed to obtain sequence reads. The sequence reads can include ending sequences corresponding to ends of the plurality of cell-free DNA fragments. Block 5710 may be performed in a similar manner as block 4610.

At block 5720, for each of the plurality of cell-free DNA fragments, a pair of sequence motifs for the ending sequences of the cell-free DNA fragment is determined. Block 4620 may be performed in a similar manner as block 5720.

At block 5730, one or more relative frequencies of a set of one or more sequence motif pairs corresponding to the ending sequences of the plurality of cell-free DNA fragments are determined. A relative frequency of a sequence motif pair can provide a proportion of the plurality of cell-free DNA fragments that have a pair of ending sequences corresponding to the sequence motif pair. Block 5730 may be performed in a similar manner as block 4630.

The set of one or more sequence motif pairs can be identified using a reference set of one or more reference samples for which a fractional concentration is known. The fractional concentration of clinically-relevant DNA may be determined using genotypic differences. Differences between the end motif pairs of the clinically-relevant DNA and the other DNA (e.g., DNA from a healthy individual, DNA from a pregnant woman (also referred as maternal DNA), or DNA of a subject who received a transplanted organ) may be determined, and used in combination with the fractional concentrations. Particular end motif pairs can be selected on the basis of the differences in the relative frequencies correlating with the differences in the fractional concentrations of the reference samples. An end motif pair with the best correlation (e.g. as measured by a goodness of fit, such as R) can be used. If an end motif pair has a low frequency, more end motif pairs can be added to the set to increase the statistical accuracy for a given sample size (e.g., number of DNA fragments). If end motif pairs are combined, they should all have a same correlation, e.g., proportional or inversely proportional.

At block 5740, an aggregate value of the one or more relative frequencies of the set of one or more sequence motif pairs is determined. If just one sequence motif pair is used, the aggregate value may be the relative frequency of that one sequence motif pair. Other example aggregate values are described in block 4640 and throughout this disclosure.

At block 5750, a classification of the fractional concentration of clinically-relevant DNA in the biological sample is determined by comparing the aggregate value to one or more calibration values. The one or more calibration values can be determined from one or more calibration samples whose fractional concentration of clinically-relevant DNA are known (e.g., measured). The comparison can be to a plurality of calibration values. The comparison can occur by inputting the aggregate value into a calibration function (e.g., line 5210 in FIG. 52A or line 5310 in FIG. 53) fit to the calibration data that provides a change in the aggregate value relative to a change in the fractional concentration of the clinically-relevant DNA in the sample. As another example, the one or more calibration values can correspond to one or more aggregate values of the relative frequencies of the set of one or more sequence motif pairs that are measured using cell-free DNA fragments in the one or more calibration samples.

A calibration value can be calculated as an aggregate value for each calibration sample. A calibration data point may be determined for each sample, where the calibration data point includes the calibration value and the measured fractional concentration for the sample. These calibration data points can be used in method 5700, or can be used to determine the final calibration data points (e.g., as defined via a functional fit). For example, a linear function could be fit to the calibration values as a function of fractional concentration. The linear function can define the calibration data points to be used in method 5700. The new aggregate value of a new sample can be used as an input to the function as part of the comparison to provide an output fractional concentration. Accordingly, the one or more calibration values can be a plurality of calibration values of a calibration function that is determined using fractional concentrations of clinically-relevant DNA of a plurality of calibration samples.

As another example, the new aggregate value can be compared to an average aggregate value for samples having a same classification of fractional concentrations (e.g., in a same range). If the new aggregate value is closer to this average than a calibration value for the average for another classification, the new sample can be determined to have a same concentration as the closest calibration value. Such a technique may be used when clustering is performed. For example, the calibration value can be a representative value for a cluster that corresponds to a particular classification of the fractional concentration.

The determination of a calibration data point can include measuring a fractional concentration, e.g., as follows. For each calibration sample of the one or more calibration samples, the fractional concentration of clinically-relevant DNA can be measured in the calibration sample. The aggregate value of the relative frequencies of the set of one or more sequence motif pairs can be determined by analyzing cell-free DNA fragments from the calibration sample as part of obtaining a calibration data point, thereby determining one or more aggregate values. Each calibration data point can specify the measured fractional concentration of clinically-relevant DNA in the calibration sample and the aggregate value determined for the calibration sample. The one or more calibration values can be the one or more aggregate values or be determined using the one or more aggregate values (e.g., when using a calibration function).

The measurement of the fractional concentration can be performed in various ways as described herein, e.g., by using an allele specific to the clinically-relevant DNA. In various embodiments, measuring a fractional concentration of clinically-relevant DNA can be performed using a tissue-specific allele or epigenetic marker, or using a size of DNA fragments, e.g., as described in US Patent Publication 2013/0237431, which is incorporated by reference in its entirety. Tissue-specific epigenetic markers can include DNA sequences that exhibit tissue-specific DNA methylation patterns in the sample.

In various embodiments, the clinically-relevant DNA can be selected from a group consisting of fetal DNA, tumor DNA, DNA from a transplanted organ, and a particular tissue type (e.g., from a particular organ). The clinically-relevant DNA can be of a particular tissue type, e.g., the particular tissue type is liver or hematopoietic. When the subject is a pregnant female, the clinically-relevant DNA can be placental tissue, which corresponds to fetal DNA. As another example, the clinically-relevant DNA can be tumor DNA derived from an organ that has cancer.

### VIII. Classification and Calibration

The classification for pathology and fractional concentration of clinically-relevant DNA can be performed in various ways. Further details are provided below. And further details are provided for the calibration of reference values, reference patterns of samples with known classifications (e.g., fractional concentration or known level of pathology), and uses of such in machine learning models.

A. Classification Techniques

As described above, various classification techniques can be used, and the aggregate value can be determined in various ways. For example, a vector comprising relative frequencies of different end motif pairs can be determined, e.g., specified as (0.8%, 4%, 2%, . . . ), which form a pattern of N relative frequencies of N different set of end motif pair(s). Each sample in a training set can correspond to a vector defining a multidimensional data point or reference pattern. Example clustering techniques include, but not limited to, hierarchical clustering, centroid-based clustering, distribution-based clustering, density-based clustering. The different clusters can correspond to differing levels of pathology or amounts of the clinically-relevant DNA in the sample, as those will have different patterns of relative frequencies, due to the differences in frequency of end motif pairs between two types of DNA fragments (e.g., maternal and fetal DNA fragments).

Accordingly, a machine learning (e.g., deep learning) models can be used for training a classifier (e.g., a cancer classifier) by making use an N-dimensional vector comprising the relative frequencies of N plasma DNA end motif pairs, including but not limited to support vector machines (SVM), decision tree, naive Bayes classification, logistic regression, clustering algorithm, principal component analysis (PCA), singular value decomposition (SVD), t-distributed stochastic neighbor embedding (tSNE), artificial neural network, as well as ensemble methods which construct a set of classifiers and then classify new data points by taking a weighted vote of their predictions. Once the classifier is trained based on an “N-dimensional vector based matrix” including a series of cancer patients and non-cancer patients, the probability of being cancer for a new patient would be able to be predicted.

In such uses of machine learning algorithms, the aggregate value can correspond to a probability or a distance (e.g., when using SVMs) that can be compared to a reference value. In other embodiments, the aggregate value can correspond to an output earlier in the model (e.g., an earlier layer in a neural network) that is compared to a cutoff between two classifications or compared to a representative value of a given classification.

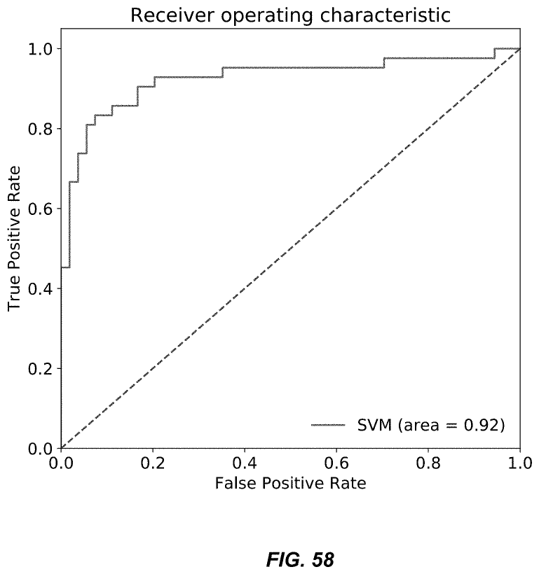

FIG. 58 shows an ROC curve for SVM modeling using end motif pairs of −1 and +1 position nucleotides to distinguish non-cancer and HCC subjects according to embodiments of the present disclosure. The same data set as section II is used. An AUC of 0.92 is achieved, which is just above the AUC of C< >C (0.91 in FIG. 7C), just below the AUC of AG< >TA (0.938 in FIG. 14A), and about the same as the AUC of t|C< >c|C (0.0917 in FIGS. 19A and 19C)

The feature vector for the SVM model includes the relative frequency of each of the 256 combinations for the fragment type of end2:−1+1. Support vector machines were used to separate the non-cancer and HCC subjects. In other implementations, only a portion of all the possible combinations can be used. For example, the top 20, 30, 50, etc. end motif pairs (e.g., as measured by AUC) can be used.

B. Calibration Function

As described herein, the reference values can be determined using one or more reference (calibration) samples that have a known classification. For example, the reference samples can be known to be healthy or known to have a pathology. As other examples, the reference/calibration samples can have known or measured fractional concentration of clinically-relevant DNA for a given calibration value (e.g., a parameter including any of the amounts described herein).

The one or more calibration values can be one or more reference values or be used to determine a reference value. The reference values can correspond to particular numerical values for the classifications. For example, calibration data points (calibration value and measured property, such as nuclease activity or level of efficacy) can be analyzed via interpolation or regression to determine a calibration function (e.g., a linear function). Then, a point of the calibration function can be used to determine the numerical classification as an input based on the input of the measured amount or other parameter (e.g., a separation value between two amounts or between a measured amount and a reference value). Such techniques may be applied to any of the method described herein.

For an example with method 5700, the reference value can be determined using one or more reference samples having a known or measured classification for the pathology or fractional concentration, respectively. The corresponding aggregate value (e.g., the value in block 4640 or 5740) can be measured in the one or more reference samples, thereby providing calibration data points comprising the two measurements for the reference/calibration samples. The one or more reference samples can be a plurality of reference samples. A calibration function can be determined that approximates calibration data points corresponding to the measured efficacies and measured amounts for the plurality of reference samples, e.g., by interpolation or regression.

### IX. Filtering and Enrichment

The preference of DNA fragments from particular tissue to exhibit a particular set of end motif pairs can be used to enrich a sample for DNA from that particular tissue. Accordingly, embodiments can enrich a sample for clinically-relevant DNA. For example, only DNA fragments having a particular pair of ending sequences may be sequenced, amplified, and/or captured using an assay. As another example, filtering of sequence reads can be performed.

A. Filtering for improved discrimination

Certain criteria can be used to filter specific DNA fragments (besides by end motif pairs) to provide greater accuracy, e.g., sensitivity and specificity. As examples, the biterminal analysis can be restricted to DNA fragments that originate from open chromatin regions of a particular tissue, e.g., as determined by reads aligning entirely within or partially to one of a plurality of open chromatin regions. For example, any read with at least one nucleotide overlapping with an open chromatin region can be defined as a read within an open chromatin region. The typical open chromatin region is about 300 bp according to DNase I hypersensitive site. The size of an open chromatin region can variable, depending on the technique used to define the open chromatin regions, for example, ATAC-seq (Assay for Transposase Accessible Chromatin sequencing) vs. DNaseI-Seq.

As another example, DNA fragments of a particular size can be selected for performing the end motif analysis. This can increase the separation of an aggregate value of relative frequencies of end motifs, thereby increasing accuracy. For example, DNA fragments less than a specified length, mass, or weight can be kept and larger/longer fragments can be discarded. As examples, size cutoffs can be 150 bp, 200 bp, 250 bp, 300 bp, etc. Such size sampling can performed in silico or by a physical process, such as electrophoresis.

A further example can use methylation properties of the DNA fragments. Fetal and tumor DNA molecules are generally hypomethylated. A fetal analysis may be used for determining fractional concentrations of clinically-relevant DNA. Embodiments can determine a methylation metric (e.g., density) of a DNA fragment (e.g., as a proportion or absolute number of site(s) that are methylated on a DNA fragment). DNA fragments can be selected for use in the biterminal analysis based on the measured methylation densities. For example, a DNA fragment can be used only if the methylation density is above a threshold.

Whether a DNA fragment includes a sequence variation (e.g. base substitution, insertion, or deletion) relative to a reference genome can also be used for filtering.

The various filtering criteria can be used in combination together. For example, each criterion may need to be satisfied, or at least a specific number of criteria may need to be satisfied. In another implementation, a probability that a fragment corresponds to clinically-relevant DNA (e.g., fetal, tumor, or transplant) can be determined, and a threshold imposed for the probability, for which a DNA fragment is to satisfy before being used in a biterminal analysis. As a further example, a contribution of a DNA fragment to a frequency counter of a particular end motif pair can be weighted based on the probability (e.g., adding the probability that has a value less than one, instead of adding one). Thus, DNA fragments with particular end motif pair(s) would be weighted higher and/or have a higher probability. Such enrichment is described further below.

B. Physical Enrichment

Physical enrichment may be performed in various ways, e.g., via targeted sequencing or PCR, as may be performed using particular primers or adapters. If a particular end motif pair is detected, then an adaptor can be added to the end of the fragment. Then, when sequencing is performed, only DNA fragments with the adapter will be sequenced (or at least predominantly sequenced), thereby providing targeted sequencing.

As another example, primers that hybridize to the particular set of end motif pairs can be used. Then, sequencing or amplification can be performed using these primers. Capture probes corresponding to the particular end motif pairs can also be used to capture DNA molecules with those end motif pairs for further analysis. Some embodiments can ligate a short oligonucleotide to the ends of a plasma DNA molecule. Then, a probe can be designed such that it would only recognize a sequence that is partially the end motif and partially the ligated oligonucleotide, with a particular pair of probes corresponding to the particular end motif pair.

Some embodiments can use clustered regularly interspaced short palindromic repeats (CRISPR)-based diagnostic technology, e.g. using a guide RNA to localize a site corresponding to a preferred end motif for the clinically-relevant DNA and then a nuclease to cut the DNA fragment, as may be done using CRISPR-associated protein 9 (Cas9) or CRISPR-associated protein 12 (Cas12). For example, an adapter can be used to recognize each end motif of the pair, and then CRISPR/Cas9 or Cas12 can be used to cut the end motif/adaptor hybrid and create a universal recognizable end for further enrichment of the molecules with the desired ends.

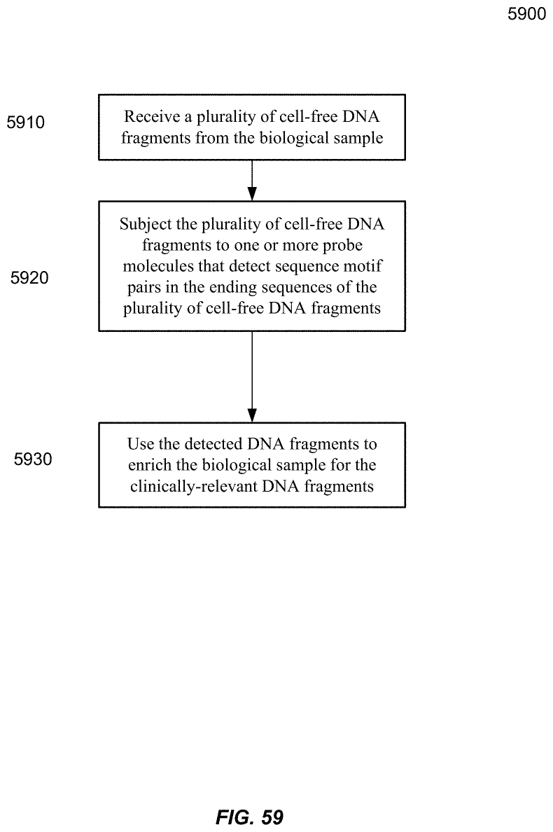

FIG. 59 is a flowchart illustrating a method 5900 of physically enriching a biological sample for clinically-relevant DNA according to embodiments of the present disclosure. The biological sample includes the clinically-relevant DNA molecules and other DNA molecules that are cell-free. Method 5900 can use particular assays to perform the enrichment.

At block 5910, a plurality of cell-free DNA fragments from the biological sample is received. The clinically-relevant DNA fragments (e.g., fetal or tumor) have ending sequences of sequence motif pairs that occur at a relative frequency greater than the other DNA (e.g., maternal DNA, healthy DNA, or blood cells). As examples, data from FIGS. 3 and 13 can be used). Thus, the sequence motif pairs can be used to enrich for the clinically-relevant DNA.

At block 5920, the plurality of cell-free DNA fragments is subjected to one or more probe molecules that detect the sequence motif pairs in the ending sequences of the plurality of cell-free DNA fragments. Such use of probe molecules can result in obtaining detected DNA fragments. In one example, the one or more probe molecules can include one or more enzymes that interrogate the plurality of cell-free DNA fragments and that append a new sequence that is used to amplify the detected DNA fragments. In another example, the one or more probe molecules can be attached to a surface for detecting the sequence motif pairs in the ending sequences by hybridization.

At block 5930, the detected DNA fragments are used to enrich the biological sample for the clinically-relevant DNA fragments. As an example, using the detected DNA fragments to enrich the biological sample for the clinically-relevant DNA fragments can includes amplifying the detected DNA fragments. As another example, the detected DNA fragments can be captured, and non-detected DNA fragments can be discarded.

C. In Silico Enrichment

The in silico enrichment can use various criteria to select or discard certain DNA fragments. Such criteria can include end motif pairs, open chromatin regions, size, sequence variation, methylation and other epigenetic characteristics. Epigenetic characteristics include all modifications of the genome that do not involve a change in DNA sequence. The criteria can specify cutoffs, e.g., requiring certain properties, such as a particular size range, methylation metric above or below a certain amount, combination of methylation status (methylated or unmethylated) of more than one CpG sites (e.g., a methylation haplotype (Guo et al, Nat Genet. 2017; 49: 635-42)), etc., or having a combined probability above a threshold. Such enrichment can also involve weighting DNA fragments based on such a probability.

As examples, the enriched sample can be used to classify a pathology (as described above), as well as to identify tumor or fetal mutations or for tag-counting for amplification/deletion detection of a chromosome or chromosomal region. For instance, if a particular end motif pair is associated with liver cancer (i.e., a higher relative frequency than for non-cancer or other cancers), then embodiments for performing cancer screening can weight such DNA fragments higher than DNA fragments not having this preferred one or this preferred set of end motifs.

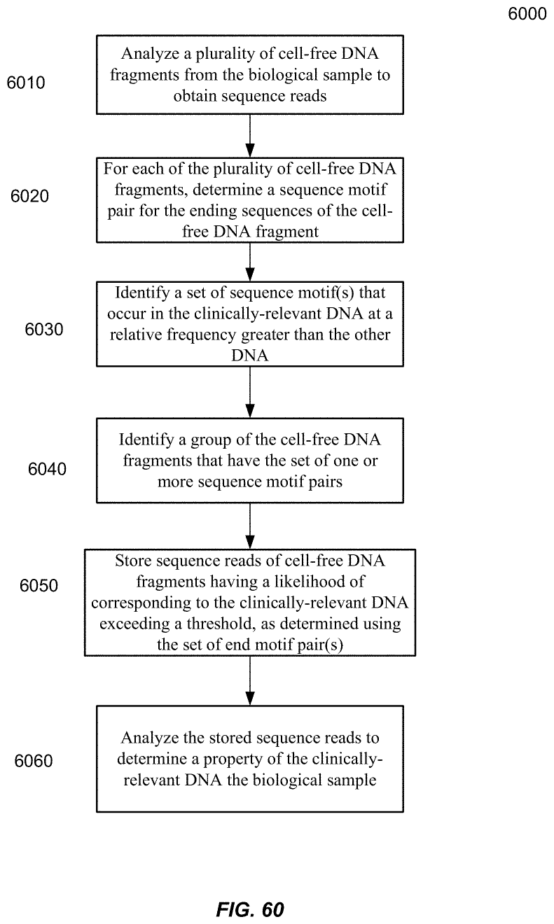

FIG. 60 is a flowchart illustrating a method for in silico enriching of a biological sample for clinically-relevant DNA according to embodiments of the present disclosure.

The biological sample includes the clinically-relevant DNA molecules and other DNA molecules that are cell-free. Method 6000 can use particular criteria of sequence reads to perform the enrichment.

At block 6010, a plurality of cell-free DNA fragments from the biological sample is analyzed to obtain sequence reads. The sequence reads include ending sequences corresponding to ends of the plurality of cell-free DNA fragments. Block 6010 may be performed in a similar manner as block 4610 of FIG. 46.

At block 6020, for each of the plurality of cell-free DNA fragments, a sequence motif pair is determined for the ending sequences of the cell-free DNA fragment. Block 6020 may be performed in a similar manner as block 4620 of FIG. 46.

At block 6030, a set of one or more sequence motif pairs that occur in the clinically-relevant DNA at a relative frequency greater than the other DNA is identified. The set of sequence motif pair(s) can be identified by genotypic or phenotypic techniques described herein. Calibration or references samples may be used to rank and select sequence motif pairs that are selective for the clinically-relevant DNA.

At block 6040, a group of the plurality of cell-free DNA fragments that have the set of one or more sequence motif pairs is identified. This can be viewed as a first stage of filtering.

At block 6050, cell-free DNA fragments having a likelihood of corresponding to the clinically-relevant DNA exceeding a threshold can be stored. The likelihood can be determined using the set of end motif pair(s). For instance, for each cell-free DNA fragment of the group of the cell-free DNA fragments, a likelihood that the cell-free DNA fragment corresponds to the clinically-relevant DNA can be determined based on the ending sequences including a sequence motif pair of the set of sequence motif pair(s). The likelihood can be compared to a threshold. As an example, a suitable threshold can be determined empirically. For instance, various thresholds can be tested for samples having a known marker for the clinically-relevant DNA. A resulting concentration of the clinically-relevant DNA can be determined for each threshold.

An optimal threshold can maximize the concentration while maintaining a certain percentage of the total number of sequence reads. The threshold could be determined by one or more given percentiles (5^{th}, 10^{th}, 90^{th}, or 95^{th}) of the concentrations of one or more end motif pairs present in the healthy controls or in control groups exposed to similar etiological risk factors but without diseases. The threshold could be a regression or probabilistic score.

The sequence read(s) can be stored in memory (e.g., in a file, table, or other data structure) when the likelihood exceeds the threshold, thereby obtaining stored sequence reads. Sequence reads of cfDNA having a likelihood below the threshold can be discarded or not stored in the memory location of the reads that are kept, or a field of a database can include a flag indicating the read had a lower threshold so that later analysis can exclude such reads. As examples, the likelihood can be determined using various techniques, such as odds ratio, z-scores, or probability distributions.

At block 6060, the stored sequence reads can be analyzed to determine a property of the clinically-relevant DNA the biological sample, e.g., as described herein, such as described in other flowcharts. Methods 4600 and 5700 are such examples. For instance, the property of the clinically-relevant DNA the biological sample can be a fractional concentration of the clinically-relevant DNA. As another example, the property can be a level of pathology of a subject from whom the biological sample was obtained, where the level of pathology is associated with the clinically-relevant DNA.

Other criteria can be used to determine the likelihood. Sizes of the plurality of cell-free DNA fragments can be measured using the sequence reads. The likelihood that a particular sequence read corresponds to the clinically-relevant DNA can be further based on a size of the cell-free DNA fragment corresponding to the particular sequence read.

Methylation can also be used. Thus, embodiments can measure one or more methylation statuses at one or more sites of a cell-free DNA fragment corresponding to a particular sequence read. The likelihood that the particular sequence read corresponds to the clinically-relevant DNA can be further based on the one or more methylation statuses. As a further example, whether a read is within an identified set of open chromatin regions can be used as a filter.

For any of the methods described herein, the sequence motif pair of the cell-free DNA fragment can be performed using a reference genome (e.g., via technique 160 of FIG. 1). Such a technique can include: aligning one or more sequence reads corresponding to the cell-free DNA fragment to a reference genome, identifying one or more bases in the reference genome that are adjacent to the ending sequence, and using the ending sequence and the one or more bases to determine the sequence motif pair.

### X. Treatment

Embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### XI. Example Systems

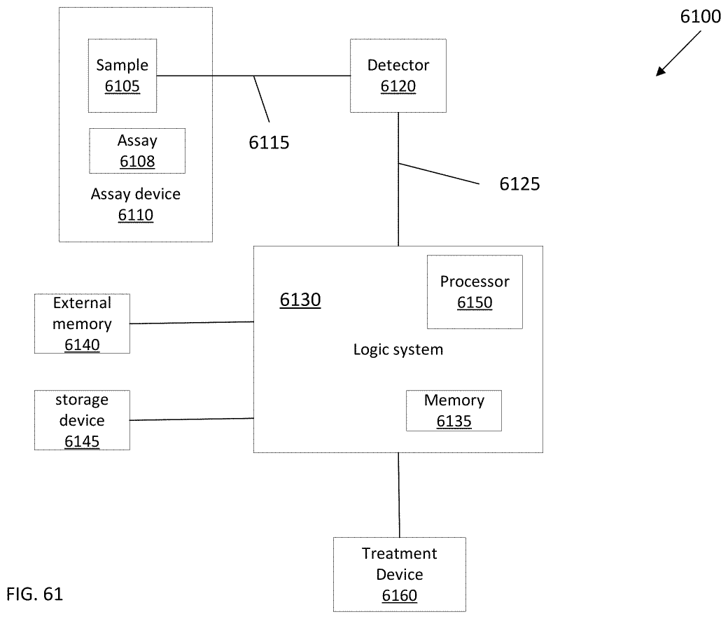

FIG. 61 illustrates a measurement system 6100 according to an embodiment of the present disclosure. The system as shown includes a sample 6105, such as cell-free DNA molecules within an assay device 6110, where an assay 6108 can be performed on sample 6105. For example, sample 6105 can be contacted with reagents of assay 6108 to provide a signal of a physical characteristic 6115. An example of an assay device can be a flow cell that includes probes and/or primers of an assay or a tube through which a droplet moves (with the droplet including the assay). Physical characteristic 6115 (e.g., a fluorescence intensity, a voltage, or a current), from the sample is detected by detector 6120. Detector 6120 can take a measurement at intervals (e.g., periodic intervals) to obtain data points that make up a data signal. In one embodiment, an analog-to-digital converter converts an analog signal from the detector into digital form at a plurality of times. Assay device 6110 and detector 6120 can form an assay system, e.g., a sequencing system that performs sequencing according to embodiments described herein. A data signal 6125 is sent from detector 6120 to logic system 6130. As an example, data signal 6125 can be used to determine sequences and/or locations in a reference genome of DNA molecules. Data signal 6125 can include various measurements made at a same time, e.g., different colors of fluorescent dyes or different electrical signals for different molecule of sample 6105, and thus data signal 6125 can correspond to multiple signals. Data signal 6125 may be stored in a local memory 6135, an external memory 6140, or a storage device 6145.

Logic system 6130 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 6130 and the other components may be part of a stand-alone or network connected computer system, or they may be directly attached to or incorporated in a device (e.g., a sequencing device) that includes detector 6120 and/or assay device 6110. Logic system 6130 may also include software that executes in a processor 6150. Logic system 6130 may include a computer readable medium storing instructions for controlling measurement system 6100 to perform any of the methods described herein. For example, logic system 6130 can provide commands to a system that includes assay device 6110 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 6100 may also include a treatment device 6160, which can provide a treatment to the subject. Treatment device 6160 can determine a treatment and/or be used to perform a treatment. Examples of such treatment can include surgery, radiation therapy, chemotherapy, immunotherapy, targeted therapy, hormone therapy, and stem cell transplant. Logic system 6130 may be connected to treatment device 6160, e.g., to provide results of a method described herein. The treatment device may receive inputs from other devices, such as an imaging device and user inputs (e.g., to control the treatment, such as controls over a robotic system).

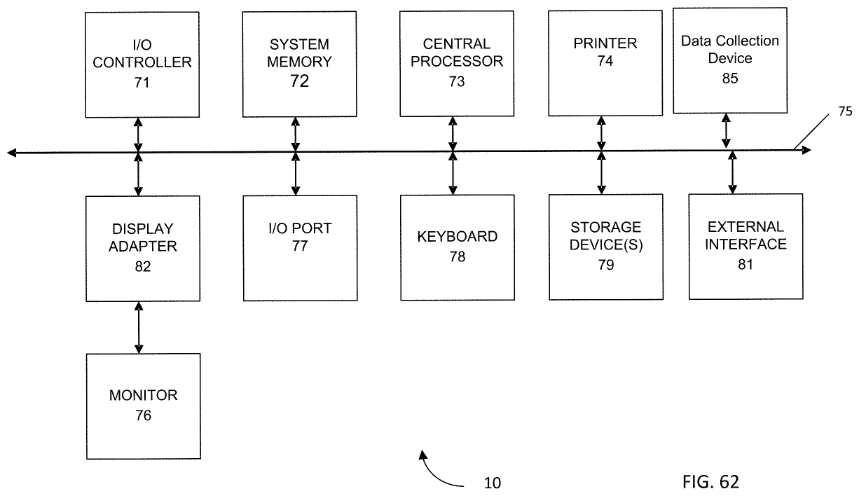

Any of the computer systems mentioned herein may utilize any suitable number of subsystems. Examples of such subsystems are shown in FIG. 62 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 63 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order that is logically possible. Additionally, portions of these steps may be used with portions of other steps from other methods. Also, all or portions of a step may be optional. Additionally, any of the steps of any of the methods can be performed with modules, units, circuits, or other means of a system for performing these steps.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The above description of example embodiments of the present disclosure has been presented for the purposes of illustration and description and are set forth so as to provide those of ordinary skill in the art with a complete disclosure and description of how to make and use embodiments of the present disclosure. It is not intended to be exhaustive or to limit the disclosure to the precise form described nor are they intended to represent that the experiments are all or the only experiments performed. Although the disclosure has been described in some detail by way of illustration. and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the disclosure being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element Which may be optional. As such, this statement is intended to serve as antecedent basis for use of such exclusive terminology as “solely”, “only”, and the like in connection with the recitation of claim. elements, or the use of a “negative” limitation.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with Which the publications are cited. None is admitted to be prior art.

### XII. REFERENCES

1. Chan K C A, Woo J K S, King A, Zee B C Y, Lam W K J, Chan S L, et al. Analysis of Plasma Epstein-Barr Virus DNA to Screen for Nasopharyngeal Cancer. N Engl J Med [Internet]. 2017/08/10. 2017; 377(6):513-22. Available from: https://www.nejm.org/doi/pdf/10.1056/NEJMoa1701717
2. Chiu R W K, Chan K C A, Gao Y, Lau V Y M, Zheng W, Leung T Y, et al. Noninvasive prenatal diagnosis of fetal chromosomal aneuploidy by massively parallel genomic sequencing of DNA in maternal plasma. Proc Natl Acad Sci USA [Internet]. 2008; 105(51):20458-63. Available from: http://www.pnas.org/content/105/51/20458.abstract
3. Lo Y M D, Corbetta N, Chamberlain P F, Rai V, Sargent I L, Redman C W G, et al. Presence of fetal DNA in maternal plasma and serum. Lancet [Internet]. 1997; 350(9076):485-7. Available from: http://dx.doi.org/10.1016/S0140-6736(97)02174-0
4. Lo Y M D, Chan K C A, Sun H, Chen E Z, Jiang P, Lun F M F, et al. Maternal Plasma DNA Sequencing Reveals the Genome-Wide Genetic and Mutational Profile of the Fetus. Sci Transl Med [Internet]. 2010; 2(61):61ra91-61ra91. Available from: http://stm.sciencemag.org/content/scitransmed/2/61/61ra91.full.pdf
5. Chandrananda D, Thorne N P, Bahlo M. High-resolution characterization of sequence signatures due to non-random cleavage of cell-free DNA. BMC Med Genomics [Internet]. 2015/06/18. 2015 [cited 2019 Dec. 31]; 8(1):29. Available from: https://doi.org/10.1186/s12920-015-0107-z
6. Ivanov M, Baranova A, Butler T, Spellman P, Mileyko V. Non-random fragmentation patterns in circulating cell-free DNA reflect epigenetic regulation. BMC Genomics [Internet]. 2015; 16(13):S1. Available from: https://doi.org/10.1186/1471-2164-16-S13-S1
7. Snyder M W, Kircher M, Hill A J, Daza R M, Shendure J. Cell-free DNA Comprises an In Vivo Nucleosome Footprint that Informs Its Tissues-Of-Origin. Cell [Internet]. 2016/01/16. 2016; 164(1-2):57-68. Available from: https://ac.els-cdn.com/S009286741501569X/1-s2.0-5009286741501569X-main.pdf?_tid=7ad5c682-f178-4148-9ef5-5155f3622c97&acdnat=1544003447 49d657134037d6cfe06c891e02a8b96e
8. Sun K, Jiang P, Cheng S H, Cheng T H T, Wong J, Wong V W S, et al. Orientation-aware plasma cell-free DNA fragmentation analysis in open chromatin regions informs tissue of origin. Genome Res [Internet]. 2019; 29(3):418-27. Available from: http://genome.cshlp.org/content/29/3/418.abstract 9. Jiang P, Sun K, Tong Y K, Cheng S H, Cheng T H T, Heung M M S, et al. Preferred end coordinates and somatic variants as signatures of circulating tumor DNA associated with hepatocellular carcinoma. Proc Natl Acad Sci USA [Internet]. 2018/10/31. 2018; 115(46):E10925-e10933. Available from: http://www.pnas.org/content/pnas/115/46/E10925.full.pdf

1. A method of analyzing a biological sample of a subject, the biological sample including cell-free DNA, the method comprising:
analyzing a plurality of cell-free DNA fragments from the biological sample to obtain sequence reads, wherein the sequence reads include ending sequences corresponding to ends of the plurality of cell-free DNA fragments;
for each of the plurality of cell-free DNA fragments, determining a pair of sequence motifs for the ending sequences of the cell-free DNA fragment;
determining one or more relative frequencies of a set of one or more sequence motif pairs corresponding to the ending sequences of the plurality of cell-free DNA fragments, wherein a relative frequency of a sequence motif pair provides a proportion of the plurality of cell-free DNA fragments that have a pair of ending sequences corresponding to the sequence motif pair;
determining an aggregate value of the one or more relative frequencies of the set of one or more sequence motif pairs; and
determining a classification of a level of pathology for the subject based on a comparison of the aggregate value to a reference value.

2. The method of claim 1, further comprising:
filtering the cell-free DNA using one or more criteria to identify the plurality of cell-free DNA fragments.

3. The method of claim 1, wherein the pathology is HBV or cirrhosis.

4. The method of claim 1, wherein the pathology is an auto-immune disorder.

5. The method of claim 4, wherein the auto-immune disorder is systemic lupus erythematosus.

6. The method of claim 1, wherein the pathology is a cancer.

7. The method of claim 6, wherein the cancer is hepatocellular carcinoma, lung cancer, breast cancer, gastric cancer, glioblastoma multiforme, pancreatic cancer, colorectal cancer, nasopharyngeal carcinoma, and head and neck squamous cell carcinoma.

8. The method of claim 6, wherein the classification is determined from a plurality of levels of cancer that include a plurality of stages of cancer.

9. The method of claim 6, wherein the classification is that the subject has cancer, wherein the method further comprises:
determining one or more additional relative frequencies of a set of one or more additional sequence motif pairs corresponding to the ending sequences of the plurality of cell-free DNA fragments;
determining an additional aggregate value of the one or more additional relative frequencies of the set of one or more additional sequence motif pairs; and
determining a stage of the cancer for the subject based on a comparison of the additional aggregate value to an additional reference value.

10. The method of claim 1, wherein the set of one or more sequence motif pairs includes a plurality of sequence motifs, wherein the one or more relative frequencies include a plurality of relative frequencies, and wherein determining the aggregate value of the plurality of relative frequencies includes determining a difference between each of the plurality of relative frequencies and a reference frequency of a reference pattern, and wherein the aggregate value includes a sum of the differences.

11. The method of claim 10, wherein the reference frequencies of the reference pattern are determined from one or more reference samples having a known classification.

12. A method of estimating a fractional concentration of clinically-relevant DNA in a biological sample of a subject, the biological sample including the clinically-relevant DNA and other DNA that are cell-free, the method comprising:
analyzing a plurality of cell-free DNA fragments from the biological sample to obtain sequence reads, wherein the sequence reads include ending sequences corresponding to ends of the plurality of cell-free DNA fragments;
for each of the plurality of cell-free DNA fragments, determining a pair of sequence motifs for the ending sequences of the cell-free DNA fragment;
determining one or more relative frequencies of a set of one or more sequence motif pairs corresponding to the ending sequences of the plurality of cell-free DNA fragments, wherein a relative frequency of a sequence motif pair provides a proportion of the plurality of cell-free DNA fragments that have a pair of ending sequences corresponding to the sequence motif pair;
determining an aggregate value of the one or more relative frequencies of the set of one or more sequence motif pairs; and
determining a classification of the fractional concentration of clinically-relevant DNA in the biological sample by comparing the aggregate value to one or more calibration values determined from one or more calibration samples whose fractional concentration of clinically-relevant DNA are known.

13-22. (canceled)

23. The method of claim 1, wherein the set of one or more sequence motif pairs are a top L sequence motif pairs with a largest difference between two types of DNA as determined in one or more reference samples, M being an integer equal to or greater than one.

24. (canceled)

25. The method of claim 23, wherein the two types of DNA are from two references samples having different classifications for the level of pathology.

26. The method of claim 1, wherein the set of one or more sequence motif pairs are a top J most frequent sequence motif pairs occurring in one or more reference samples, J being an integer equal to or greater than one.

27. The method of claim 1, wherein the set of one or more sequence motif pairs includes a plurality of sequence motif pairs, and wherein the aggregate value includes a sum of the relative frequencies of the set.

28. The method of claim 27, wherein the sum is a weighted sum.

29. The method of claim 1, wherein the classification is a first classification, wherein the method further comprises:
determining one or more additional classifications for one or more additional sets of sequence motif pairs; and
determining a final classification using the first classification and one or more additional classifications.

30. The method of claim 1, wherein the aggregate value includes a final or intermediate output of a machine learning model.

31. (canceled)

32. A method of enriching a biological sample for clinically-relevant DNA, the biological sample including the clinically-relevant DNA and other DNA that are cell-free, the method comprising:
analyzing a plurality of cell-free DNA fragments from the biological sample to obtain sequence reads, wherein the sequence reads include ending sequences corresponding to ends of the plurality of cell-free DNA fragments;
for each of the plurality of cell-free DNA fragments, determining a sequence motif pair for the ending sequences of the cell-free DNA fragment;
identifying a set of one or more sequence motif pairs that occur in the clinically-relevant DNA at a relative frequency greater than the other DNA;
identifying a group of the plurality of cell-free DNA fragments that have the set of one or more sequence motif pairs;
for each of the group of cell-free DNA fragments:
determining a likelihood that the cell-free DNA fragment corresponds to the clinically-relevant DNA based on the ending sequences including a sequence motif pair of the set of one or more sequence motif pairs;
comparing the likelihood to a threshold; and
storing the sequence read(s) of the cell-free DNA fragment when the likelihood exceeds the threshold, thereby obtaining stored sequence reads; and
analyzing the stored sequence reads to determine a property of the clinically-relevant DNA the biological sample.

33-46. (canceled)
